(19) United States
(12) Patent Application Publication
(10) Pub. No.: US 2018/0050009 A1
(43) Pub. Date: Feb. 22, 2018

(54) METHODS AND COMPOSITIONS FOR THE TREATMENT OF WARTS

(71) Applicant: ACLARIS THERAPEUTICS, INC., Malvern, PA (US)

(72) Inventors: Stuart D. SHANLER, Malvern, PA (US); Christopher POWALA, Radnor, PA (US); Christopher PHILLIPS, Doylestown, PA (US); Brian BEGER, Newtown, PA (US); Charles Rodney GREENAWAY EVANS, Surrey (GB); Sian Tiong LIM, Surrey (GB); Marc Barry BROWN, Watford Hertfordshire (GB)

(21) Appl. No.: 15/679,598

(22) Filed: Aug. 17, 2017

Related U.S. Application Data
(60) Provisional application No. 62/376,326, filed on Aug. 17, 2016.

(51) Int. Cl. A61K 31/327; A61K 47/10; A61K 9/00

(52) U.S. Cl. CPC A61K 31/327; A61K 9/0014; A61K 47/10

(57) ABSTRACT

Embodiments are directed to compositions comprising greater than 40% w/w to about 70% w/w stabilized hydrogen peroxide. The composition may also include a surface tension modifying agent, such as 2-propanol. Such compositions may be used to treat conditions such as warts, condyloma accuminatum, molluscum contagiosum, or a combination thereof. Some embodiments also describe take home compositions, in office compositions, over-the-counter compositions, and kits for the use of such compositions.

### CROSS-REFERENCED TO RELATED APPLICATIONS

The present application claims the benefit of U.S. Provisional Patent Application 62/376,326 filed Aug. 17, 2016, entitled, “METHODS AND COMPOSITIONS FOR THE TREATMENT OF WARTS,” the content of which is incorporated herein by reference in its entirety.

### BRIEF SUMMARY

Embodiments in this disclosure are directed to a composition comprising hydrogen peroxide. In some embodiments, the composition may further include an alcohol. In some embodiments, the hydrogen peroxide is stabilized hydrogen peroxide. In some embodiments, the hydrogen peroxide may be a standard grade, food grade, chemical synthesis grade, semiconductor grade, high-test hydrogen peroxide grade, antimicrobial grade, drinking water grade, pharmaceutical grade, active pharmaceutical ingredient (API) grade, or cosmetic grade hydrogen peroxide. In some embodiments, the topical composition comprises greater than 40% w/w to about 60% w/w hydrogen peroxide. In some embodiments, the topical composition comprises about 45% w/w to about 60% w/w hydrogen peroxide. In some embodiments, the hydrogen peroxide is in an amount up to about 60% w/w of the composition. In some embodiments, the topical composition comprises greater than 40% w/w to about 70% w/w hydrogen peroxide. In some embodiments, the topical composition comprises about 45% w/w to about 70% w/w hydrogen peroxide. In some embodiments, the hydrogen peroxide is in an amount up to about 70% w/w of the composition. In some embodiments, the hydrogen peroxide is in an amount of about 60% w/w of the composition. In some embodiments, the hydrogen peroxide is in an amount of about 59% w/w of the composition. In some embodiments, the hydrogen peroxide is in an amount of about 55% w/w of the composition. In some embodiments, the hydrogen peroxide is in an amount of about 54% w/w of the composition. In some embodiments, the hydrogen peroxide is in an amount of about 50% w/w of the composition. In some embodiments, the topical composition comprises about 45% w/w hydrogen peroxide. In some embodiments, the alcohol may be selected from a primary alcohol, a secondary alcohol, a tertiary alcohol, or a combination thereof. In some embodiments, the alcohol may be selected from 2-propanol, methanol, butanol, 1-propanol, pentanol, hexanol, octanol, nonanol, decanol, 2-pentanol, 2-butanol, benzyl alcohol, ethanol, an isomer thereof, or a combination thereof. In some embodiments the alcohol is in a quantity sufficient to decrease the surface tension of the composition. In some embodiments, the alcohol is in an amount up to about 5% w/w of the composition. In some embodiments, the composition is a solution. In some embodiments, the composition is a gel formulation. In some embodiments, the composition comprises two or more separate components that may be admixed before, at, or near the time of use.

Some embodiments are directed to a topical composition comprising greater than 0% w/w to about 60% w/w hydrogen peroxide and greater than 0% w/w to about 10% w/w 2-propanol. Some embodiments are directed to a topical composition comprising greater than 0% w/w to about 55% w/w hydrogen peroxide and greater than 0% w/w to about 10% w/w 2-propanol. Some embodiments are directed to a topical composition comprising greater than 0% w/w to about 54% w/w hydrogen peroxide and greater than 0% w/w to about 10% w/w 2-propanol. Some embodiments are directed to a topical composition comprising greater than 0% w/w to about 50% w/w hydrogen peroxide and greater than 0% w/w to about 10% w/w 2-propanol. Some embodiments are directed to a topical composition comprising greater than 0% w/w to about 45% w/w hydrogen peroxide and greater than 0% w/w to about 10% w/w 2-propanol. Some embodiments are directed to a topical composition comprising about 40% w/w to about 60% w/w hydrogen peroxide and greater than 0% w/w to about 10% w/w 2-propanol. Some embodiments are directed to a topical composition comprising hydrogen peroxide in an amount up to about 60% w/w and greater than 0% w/w to about 5% w/w 2-propanol. Some embodiments are directed to a topical composition comprising up to about 60% w/w hydrogen peroxide and greater than 0% w/w to about 2.5% w/w 2-propanol. In some embodiments, the composition comprises greater than 40% w/w hydrogen peroxide and greater than 0% w/w to about 10% w/w 2-propanol. In some embodiments, the composition comprises greater than 40% w/w to about 60% w/w hydrogen peroxide and greater than 0% w/w to about 10% w/w 2-propanol. In some embodiments, the composition comprises about 45% w/w hydrogen peroxide and greater than 0% w/w to about 10% w/w 2-propanol. In some embodiments, the composition comprises greater than 40% w/w hydrogen peroxide and greater than 0% w/w to about 5% w/w 2-propanol. In some embodiments, the composition comprises greater than 40% w/w hydrogen peroxide and greater than 0% w/w to about 2.5% w/w 2-propanol. In some embodiments, the composition comprises about 45% w/w hydrogen peroxide and greater than 0% w/w to about 5% w/w 2-propanol. In some embodiments, the composition comprises about 45% w/w hydrogen peroxide and about 5% w/w 2-propanol. In some embodiments, the composition comprises about 45% w/w hydrogen peroxide and about 2.5% w/w 2-propanol. In some embodiments, the 2-propanol is in an amount sufficient to decrease the surface tension of the composition. In some embodiments, the 2-propanol is in an amount sufficient to decrease the surface tension of the composition sufficient to enhance the penetration of the composition into a targeted skin area or lesion. In some embodiments the surface tension modifying agent is in a quantity sufficient to enhance the therapeutic efficacy of the composition. In some embodiments, the hydrogen peroxide is a stabilized hydrogen peroxide. In some embodiments the hydrogen peroxide is active pharmaceutical ingredient (API) grade hydrogen peroxide. In some embodiments, the topical composition further includes a stabilizer. In some embodiments, the stabilizer may be selected from a stannate, sodium pyrophosphate, organophosphonate, nitrate, phosphoric acid, colloidal silicate, any stabilizer known in the art, or a combination thereof. In some embodiments, the 2-propanol is in an amount sufficient to decrease the surface tension while minimizing spread of the composition onto non-targeted skin when applied to a targeted lesion. In some embodiments, the 2-propanol is in an amount sufficient to increase the penetration of the composition into the targeted lesion. The composition may be stable for at least about two years at 5° C. at least about 12 months at 30° C., at least about 12 months at 25° C., at least about 12 months at 5° C., at least about 6 months at 5° C., at least about 6 months at 25° C., at least about 6 months at 40° C., at least about 3 months at 5° C., at least about 3 months at 25° C., at least about 3 months at 40° C., or a combination thereof.

In some embodiments, the topical composition has a surface tension of about 42 mN·m-1 to about 55 mN·m-1 at 37° C. In some embodiments, the topical composition comprises about 60% w/w stabilized cosmetic-grade hydrogen peroxide and about 5% w/w 2-propanol, and wherein the topical composition has a surface tension of about 42 mN·m-1 to about 55 mN·m-1 at 37° C. In some embodiments, the topical composition comprises about 59% w/w stabilized cosmetic-grade hydrogen peroxide and about 5% w/w 2-propanol, and wherein the topical composition has a surface tension of about 42 mN·m-1 to about 55 mN·m-1 at 37° C. In some embodiments, the topical composition comprises about 55% w/w stabilized cosmetic-grade hydrogen peroxide and about 5% w/w 2-propanol, and wherein the topical composition has a surface tension of about 42 mN·m-1 to about 55 mN·m-1 at 37° C. In some embodiments, the topical composition comprises about 54% w/w stabilized cosmetic-grade hydrogen peroxide and about 5% w/w 2-propanol, and wherein the topical composition has a surface tension of about 42 mN·m-1 to about 55 mN·m-1 at 37° C. In some embodiments, the topical composition comprises about 50% w/w stabilized cosmetic-grade hydrogen peroxide and about 5% w/w 2-propanol, and wherein the topical composition has a surface tension of about 42 mN·m-1 to about 55 mN·m-1 at 37° C. In some embodiments, the topical composition comprises about 45% w/w stabilized cosmetic-grade hydrogen peroxide and about 5% w/w 2-propanol, and wherein the topical composition has a surface tension of about 42 mN·m-1 to about 55 mN·m-1 at 37° C. In some embodiments, the topical composition comprises about 40% w/w stabilized cosmetic-grade hydrogen peroxide and about 5% w/w 2-propanol, and wherein the topical composition has a surface tension of about 42 mN·m-1 to about 55 mN·m-1 at 37° C. In some embodiments, the topical composition comprises about 32.5% w/w stabilized cosmetic-grade hydrogen peroxide and about 5% w/w 2-propanol, and wherein the topical composition has a surface tension of about 42 mN·m-1 to about 55 mN·m-1 at 37° C. In some embodiments, the topical composition comprises about 25% w/w stabilized cosmetic-grade hydrogen peroxide and about 5% w/w 2-propanol, and wherein the topical composition has a surface tension of about 42 mN·m-1 to about 55 mN·m-1 at 37° C.

Some embodiments are directed to a composition comprising hydrogen peroxide and a surface tension modifying agent. In some embodiments, the composition comprises up to about 60% w/w hydrogen peroxide. In some embodiments, the composition comprises about 40% w/w or greater of hydrogen peroxide. In some embodiments, the composition comprises about 40% w/w to about 60% w/w hydrogen peroxide. In some embodiments, the surface tension modifying agent is an agent stable in compositions comprising concentrations of hydrogen peroxide disclosed in embodiments herein. In some embodiments, the surface tension modifying agent is in a quantity sufficient to enhance the therapeutic efficacy of the composition. In some embodiments, the surface tension modifying agent is in a quantity sufficient to modify the surface tension while maintaining stability of the composition sufficient for use as a commercially viable formulation. In some embodiments, the surface tension modifying agent is an alcohol. In some embodiments, the surface tension modifying agent is 2-propanol.

Some embodiments describe a method of treating warts comprising administering a topical composition having up to about 60% w/w hydrogen peroxide to a subject in need thereof. In some embodiments, the topical composition further comprises an alcohol. In some embodiments, the composition comprises up to about 50% w/w hydrogen peroxide. In some embodiments, the composition comprises up to about 45% w/w hydrogen peroxide. In some embodiments, the composition comprises greater than 40% w/w hydrogen peroxide. In some embodiments, the composition comprises about 40% w/w to about 60% w/w hydrogen peroxide. In some embodiments, the composition comprises greater than 40% w/w to about 60% w/w hydrogen peroxide. In some embodiments, the composition comprises about 59% w/w hydrogen peroxide. In some embodiments, the composition comprises about 55% w/w hydrogen peroxide. In some embodiments, the composition comprises about 54% w/w hydrogen peroxide. In some embodiments, the composition comprises about 45% w/w hydrogen peroxide. In some embodiments, the composition comprises about 45% w/w hydrogen peroxide and greater than 0% w/w to about 5% w/w 2-propanol. In some embodiments, the composition comprises about 45% w/w hydrogen peroxide and about 5% w/w 2-propanol. In some embodiments, the composition comprises about 45% w/w hydrogen peroxide and about 2.5% w/w 2-propanol. In some embodiments, the composition comprises about 59% w/w hydrogen peroxide and greater than 0% w/w to about 5% w/w 2-propanol. In some embodiments, the composition comprises about 59% w/w hydrogen peroxide and about 5% w/w 2-propanol. In some embodiments, the composition comprises about 59% w/w hydrogen peroxide and about 2.5% w/w 2-propanol. In some embodiments, the composition comprises about 55% w/w hydrogen peroxide and greater than 0% w/w to about 5% w/w 2-propanol. In some embodiments, the composition comprises about 55% w/w hydrogen peroxide and about 5% w/w 2-propanol. In some embodiments, the composition comprises about 55% w/w hydrogen peroxide and about 2.5% w/w 2-propanol. In some embodiments, the composition comprises about 54% w/w hydrogen peroxide and greater than 0% w/w to about 5% w/w 2-propanol. In some embodiments, the composition comprises about 54% w/w hydrogen peroxide and about 5% w/w 2-propanol. In some embodiments, the composition comprises about 54% w/w hydrogen peroxide and about 2.5% w/w 2-propanol.

In some embodiments, the warts may be a virally-induced wart. In some embodiments, the warts may be selected from common warts (verruca vulgaris), condyloma acuminata genital warts, external genital warts, palmar and plantar warts, mosaic warts, flat warts, filiform warts butcher's warts, oropharyngeal warts, anogenital warts, laryngeal warts, papillomas, dysplasias (e.g., CIN (cervical intraepithelial neoplasia)), persistent warts, periungual warts, subungual warts, recalcitrant warts, treatment naïve warts, skin lesions seen in epidermodysplasia verruciformis, or a combination thereof.

Some embodiments describe a method of treating molluscum contagiosum comprising administering a composition having up to about 60% w/w hydrogen peroxide to a subject in need thereof. In some embodiments, the composition further comprises an alcohol. In some embodiments, the composition comprises greater than 50% w/w hydrogen peroxide. In some embodiments, the composition comprises greater than 45% w/w hydrogen peroxide. In some embodiments, the composition comprises greater than 40% w/w hydrogen peroxide. In some embodiments, the composition comprises greater than 40% w/w to about 60% w/w hydrogen peroxide. In some embodiments, the composition comprises about 40% w/w to about 60% w/w hydrogen peroxide. In some embodiments, the composition comprises about 45% w/w hydrogen peroxide and greater than 0% w/w to about 5% w/w 2-propanol. In some embodiments, the composition comprises about 45% w/w hydrogen peroxide and about 5% w/w 2-propanol. In some embodiments, the composition comprises about 45% w/w hydrogen peroxide and about 2.5% w/w 2-propanol. In some embodiments, the composition comprises about 59% w/w hydrogen peroxide and greater than 0° % w/w to about 5% w/w 2-propanol. In some embodiments, the composition comprises about 59% w/w hydrogen peroxide and about 5% w/w 2-propanol. In some embodiments, the composition comprises about 59% w/w hydrogen peroxide and about 2.5% w/w 2-propanol. In some embodiments, the composition comprises about 55% w/w hydrogen peroxide and greater than 0% w/w to about 5% w/w 2-propanol. In some embodiments, the composition comprises about 55% w/w hydrogen peroxide and about 5% w/w 2-propanol. In some embodiments, the composition comprises about 55% w/w hydrogen peroxide and about 2.5% w/w 2-propanol. In some embodiments, the composition comprises about 54% w/w hydrogen peroxide and greater than 0% w/w to about 5% w/w 2-propanol. In some embodiments, the composition comprises about 54% w/w hydrogen peroxide and about 5% w/w 2-propanol. In some embodiments, the composition comprises about 54% w/w hydrogen peroxide and about 2.5% w/w 2-propanol.

In some embodiments, the alcohol is in an amount capable of decreasing the surface tension of the composition. In some embodiments, the 2-propanol is in an amount sufficient to decrease the surface tension of the composition. In some embodiments, the 2-propanol is in an amount sufficient to decrease the surface tension of the composition sufficient to enhance the penetration of the composition into a targeted skin area or lesion. In some embodiments the surface tension modifying agent is in a quantity sufficient to enhance the therapeutic efficacy of the composition. In some embodiments, the alcohol is in an amount capable of increasing the wettability of the surface of the skin or skin lesion. In some embodiments, the alcohol is in an amount capable of increasing the permeability of the composition into the subject's skin and/or the skin lesion. In some embodiments, the 2-propanol is in an amount sufficient to increase the penetration of the composition into the targeted lesion. In some embodiments, the alcohol is in an amount capable of enhancing the clinical efficacy of the composition. In some embodiments, the alcohol is in an amount capable of enhancing the clinical efficacy of the composition while maintaining stability sufficient for a commercially viable composition.

Some embodiments herein are directed to a topical solution formulation comprising up to about 60% w/w hydrogen peroxide and an alcohol. Some embodiments herein are directed to a topical solution formulation comprising greater than 40% w/w to about 60% w/w hydrogen peroxide and an alcohol. In some embodiments, the solution is comprised of two or more parts to be mixed at or immediately before the time of application.

Some embodiments herein are directed to a gel composition comprising up to about 60% w/w hydrogen peroxide, an alcohol, and a gelling agent. Some embodiments herein are directed to a gel composition comprising greater than 40% w/w to about 60% w/w hydrogen peroxide, an alcohol, and a gelling agent. In some embodiments, the gel composition is comprised of two or more parts that may be mixed at or immediately before the time of application.

Some embodiments herein are directed to a kit for treating warts comprising a container comprising greater than 40% w/w to about 60% w/w hydrogen peroxide. In some embodiments, the container further comprises greater than 0% w/w to about 5% w/w 2-propanol. In some embodiments, the 2-propanol and hydrogen peroxide are in a composition in a frangible container. In some embodiments, the 2-propanol and hydrogen peroxide are separated in the container. In some embodiments, the 2-propanol and hydrogen peroxide may be in separate containers. In some embodiments, the kit may further include a gelling agent. In some embodiments, the container further includes an applicator. In some embodiments, the container is comprised within an applicator. In some embodiments, the container is an applicator. In some embodiments, the kit further includes an applicator.

Some embodiments herein describe a composition consisting essentially of up to about 60% w/w hydrogen peroxide and an alcohol. Some embodiments herein describe a composition consisting of up to about 60% w/w hydrogen peroxide and an alcohol. In some embodiments, the alcohol is a primary alcohol, a secondary alcohol, a tertiary alcohol, or a combination thereof. In some embodiments, the alcohol is not 1-propanol, ethanol, butanol, pentanol, hexanol, octanol, nonanol, decanol, 2-butanol, 2-pentanol, or benzyl alcohol. In some embodiments, the alcohol is 2-propanol.

### DESCRIPTION OF THE FIGURES

FIG. 1 illustrates the mean cumulative amount of hydrogen peroxide released per unit area across silicone membrane (μg/cm²) following application of all 40% w/w hydrogen peroxide formulations. Each time point represents the mean±SD (n=5-6).

FIG. 2 illustrates the mean cumulative amount of hydrogen peroxide released per unit area across silicone membrane (μg/cm²) following application of 40% w/w hydrogen peroxide formulations containing 1-propanol. Each time point represents the mean±SD (n=5-6).

FIG. 3 illustrates the mean cumulative amount of hydrogen peroxide released per unit area across silicone membrane (μg/cm²) following application of 40% w/w hydrogen peroxide formulations containing 2-propanol. Each time point represents the mean±SD (n=5-6).

FIG. 4 illustrates the steady state release of hydrogen peroxide, calculated between 0.5 and 4 h (μg/cm²/h) following application of hydrogen peroxide formulations at varying levels of propanol. Each data point represents the mean±SD, n=5-6.

FIG. 5 illustrates the difference in the steady state release of hydrogen peroxide (Flux [1-propanol]-Flux [2-propanol]) for 5, 10, 15 and 20% w/w (1-propanol and 2-propanol).

FIG. 6 illustrates the mean surface tension (mN/m) of the different hydrogen peroxide formulations assessed containing either 1- or 2-propanol.

FIG. 7 illustrates the mean Wart Improvement Assessment score by visit.

FIG. 8 illustrates the mean change in baseline in the Wart Severity Assessment score by visit.

FIG. 9 illustrates the redox potential (mV) of all 40% w/w hydrogen peroxide formulations containing 1- and 2-propanol, with and without skin at t=0, 1, 6 and 24 h. Each time point represents the mean±range, n=3.

FIG. 10 illustrates the redox potential (mV) of 40% w/w hydrogen peroxide formulations containing 5% w/w 1- or 2-propanol, in with and without skin at t=0, 1, 6 and 24 h. Each time point represents the mean±range, n=3.

FIG. 11 depicts an illustrative applicator according to a first embodiment.

FIG. 12 illustrates the mean change from baseline in Physician Wart Assessment (PWA) score at Visit 10.

FIG. 13 illustrates the proportion of subjects achieving wart clearance at Visit 10.

FIG. 14 illustrates the proportion of subjects treated with the 45% w/w hydrogen peroxide solution achieving wart clearance at each post-baseline visit.

FIG. 15 illustrates the proportion of subjects treated with the 45% w/w hydrogen peroxide solution who achieved a PWA score of clear or mild at Visit 10.

FIG. 16 illustrates the proportion of subjects treated with the 45% w/w hydrogen peroxide solution who achieved a 2 ordinal reduction in PWA score at Visit 10.

FIG. 17 illustrates the local skin reactions at Visit 10 for each of the 45% w/w hydrogen peroxide solution, 40% w/w hydrogen peroxide solution, and the vehicle, where 0=no reaction; 1=mild reaction; and 2=moderate reaction.

FIG. 18 illustrates the percent of local skin reactions≦Moderate at Visit 10 for the 45% w/w hydrogen peroxide solution.

FIG. 19 illustrates the percent of local skin reactions≦Moderate at Visit 10 for the 40% w/w hydrogen peroxide solution.

FIG. 20 illustrates the percent of local skin reactions≦Moderate at Visit 10 for the vehicle.

FIG. 21 illustrates the stability (% w/w label claim) over time (in months) of a 45% w/w hydrogen peroxide and 5% w/w 2-propanol solution (2.2 ml) in 9 mm ampoules at 5° C. for 12 months.

FIG. 22 illustrates the stability (% w/w label claim) over time (in months) of a 45% w/w hydrogen peroxide and 5% w/w 2-propanol solution (2.2 ml) in 9 mm ampoules at 25° C. and 60% w/w relative humidity (RH) for 12 months.

FIG. 23 illustrates the stability (% w/w label claim) over time (in months) of a 45% w/w hydrogen peroxide and 5% w/w 2-propanol solution (2.2 ml) in 9 mm ampoules at 40° C. and 75% w/w relative humidity (RH) for 6 months.

### DETAILED DESCRIPTION

Before the present compositions and methods are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims. Unless defined otherwise, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are now described. All publications mentioned herein are incorporated by reference in their entirety. Nothing herein is to be construed as an admission that the invention is not entitled to antedate such disclosure by virtue of prior invention.

It must also be noted that as used herein and in the appended claims, the singular forms “a”, “an”, and “the” include plural reference unless the context clearly dictates otherwise. Thus, for example, reference to a “skin lesion” is a reference to one or more skin lesions and equivalents thereof known to those skilled in the art, and so forth.

As used herein, the term “about” means plus or minus 2% w/w of the numerical value of the number with which it is being used. Therefore, about 50% w/w means in the range of 49%-51% w/w.

“Administering”, when used in conjunction with a therapeutic, means to administer a therapeutic directly into or onto a target tissue or to administer a therapeutic to a subject, whereby the therapeutic positively impacts the tissue to which it is targeted. Thus, as used herein, the term “administering”, when used in conjunction with a therapeutic, can include, but is not limited to, providing a therapeutic to a subject systemically by, for example, intravenous injection, whereby the therapeutic reaches the target tissue. Administering a composition or therapeutic may be accomplished by, for example, injection, oral administration, topical administration, or by these methods in combination with other known techniques. Such combination techniques may include heating, radiation, ultrasound and the use of delivery agents. Preferably, administering is a self-administration, wherein the therapeutic or composition is administered by the subject themselves. Alternatively, administering may be administration to the subject by a health care provider.

“Providing”, when used in conjunction with a therapeutic, means to administer a therapeutic directly into or onto a target tissue, or to administer a therapeutic to a subject whereby the therapeutic positively impacts the tissue to which it is targeted.

The term “animal” as used herein includes, but is not limited to, humans and non-human vertebrates such as wild, domestic and farm animals.

The term “patient” or “subject” as used herein is an animal, particularly a human, suffering from an unwanted disease or condition that may be treated by the therapeutic and/or compositions described herein.

The term “improves” is used to convey that the embodiments provided herein change either the characteristics and/or the physical attributes of the tissue to which the therapeutic composition is being provided, applied or administered. The term “improves” may also be used in conjunction with a diseased state such that when a diseased state is “improved” the symptoms or physical characteristics associated with the diseased state are diminished, reduced or eliminated.

The term “inhibiting” generally refers to prevention of the onset of the symptoms, alleviating the symptoms, or eliminating the disease, condition or disorder.

“Optional” or “optionally” means that the subsequently described event or circumstance may or may not occur, and that the description includes instances where the event occurs and instances where it does not.

As used herein, “room temperature” means an indoor temperature of from about 20° C. to about 25° C. (68 to 77° F.).

Throughout the specification of the application, various terms are used such as “primary,” “secondary.” “first,” “second.” and the like. These terms are words of convenience in order to distinguish between different elements, and such terms are not intended to be limiting as to how the different elements may be utilized.

By “pharmaceutically acceptable,” “physiologically tolerable,” and grammatical variations thereof, as they refer to compositions, carriers, diluents, and reagents or other ingredients of the formulation, can be used interchangeably and represent that the materials are capable of being administered without the production of undesirable physiological effects such as rash, burning, irritation or other deleterious effects to such a degree as to be intolerable to the recipient thereof.

As used herein, the term “cosmetically acceptable” and grammatical variations thereof, as they refer to compositions, carriers, diluents, and reagents or other ingredients of the formulation, represent that the materials used and final composition are not irritating or otherwise harmful to the patient in general and to the skin, in particular, and preferably are pleasant and well tolerated with respect to general appearance, pH, color, smell and texture (feel), that they are not, for example, unacceptably sticky (tacky), oily or drying, and that they do spread easily, absorb into the skin at an acceptable rate of absorption, and are generally moisturizing.

As used herein, the term “therapeutic” means an agent utilized to treat, combat, ameliorate, prevent or improve an unwanted condition or disease of a subject. In part, embodiments described herein may be directed to the treatment of various skin diseases, conditions or disorders or symptoms thereof, including, but not limited to, benign proliferations, neoplasms, premalignancies or malignancies of the skin. The condition may be a wart associated condition. The condition may be a virally induced or non-virally induced cutaneous growth or proliferation. The condition may be selected from warts, common warts (verruca vulgaris), condyloma acuminata, genital warts, external genital warts, palmar and plantar warts, mosaic warts, flat warts, filiform warts butcher's warts, oropharyngeal warts, anogenital warts, laryngeal warts, papillomas, dysplasias (e.g., CIN (cervical intraepithelial neoplasia), persistent warts, periungual warts, subungual warts, recalcitrant warts, treatment naïve warts, skin lesions seen in epidermodysplasia verruciformis, molluscum contagiosum, or a combination thereof.

As used herein, the term “stabilized hydrogen peroxide” refers to a hydrogen peroxide comprising a stabilizer or a blend of stabilizers useful for dilution of the hydrogen peroxide into a concentration that can be incorporated into a stable commercial formulation for topical application to skin lesions for the treatment of skin conditions described herein. In some embodiments, the hydrogen peroxide may be obtained from a commercial source. The amount and type of stabilizer(s) used in the hydrogen peroxide formulation may be proprietary to and/or a trade secret of the commercial source. In some embodiments, the stabilized hydrogen peroxide is a hydrogen peroxide of high concentration. Though pure hydrogen peroxides of high concentration are typically stable, stabilizers may be used in hydrogen peroxide formulations, usually when obtained through commercial sources, in order to stabilize diluted versions of the “high concentration” hydrogen peroxide formulation. Some hydrogen peroxide formulations have stabilizers in concentrations (in total and/or individually) sufficient to provide stabilization of diluted hydrogen peroxide for particular uses or in particular industries. In some embodiments, the stabilized hydrogen peroxide has been approved by the Food and Drug Administration (FDA) for topical administration to humans. In some embodiments, the stabilized hydrogen peroxide has a drug master file at the FDA and been approved by the FDA for topical administration to humans.

As used herein, the term “clinical efficacy” refers to the ability of an ingredient or composition to produce a desired effect. For example, in some embodiments, the desired effect may include, without limitation, decreasing the surface tension of the composition, increasing the wettability of the surface of the skin or skin lesion, increasing the permeability of the composition into the subject's skin, skin lesion, or surface imperfections, including crevices, invaginations and irregularities of the skin or skin lesion, decreasing the size of the target lesion, improving the shape and/or appearance of the target lesion, improving the target lesion or treated area and/or removing the target lesion, or a combination thereof.

The terms “therapeutically effective” or “effective”, as used herein, may be used interchangeably and refer to an amount of a therapeutic composition of embodiments described herein. For example, a therapeutically effective amount of a composition is an amount of the composition, and particularly the active ingredient, such as hydrogen peroxide, that generally achieves the desired effect.

A “therapeutically effective amount” or “effective amount” of a composition is an amount necessary or sufficient to achieve the desired result. The activity contemplated by the embodiments herein includes medically therapeutic, cosmetically therapeutic and/or prophylactic treatment, as appropriate. The specific dose of a compound administered according to embodiments of the present invention to obtain therapeutic and/or prophylactic effects will, of course, be determined by the particular circumstances surrounding the case, including, for example, the compound administered, the route of administration, and the condition being treated. However, the effective amount administered can be determined by the practitioner or manufacturer or patient in light of the relevant circumstances including the condition to be treated, the choice of compound to be administered, and the chosen route of administration, and therefore, the above dosage ranges are not intended to limit the scope of the invention in any way. A therapeutically effective amount of the compound of embodiments herein is typically an amount such that when it is administered in a physiologically tolerable excipient composition, it is sufficient to achieve an effective systemic concentration or local concentration in or on the tissue to achieve the desired therapeutic or clinical outcome.

The terms “treat.” “treated.” or “treating” as used herein refers to therapeutic treatment, cosmetic treatment and/or prophylactic or preventative measures, wherein the object is to prevent or slow down (lessen) an undesired physiological condition, disorder or disease, or to obtain beneficial or desired clinical results. For the purposes of this disclosure, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects.

As used herein, the term “consists of” or “consisting of” means that the formulation or method includes only the elements, steps, or ingredients specifically recited in the particular claimed embodiment or claim.

As used herein, the term “consisting essentially of” or “consists essentially of” means that the formulation or method includes only the specified materials or steps and those that do not materially affect the basic and novel characteristics of the claimed invention.

Generally speaking, the term “tissue” refers to any aggregation of similarly specialized cells which are united in the performance of a particular function.

Verrucae (Warts) are virally induced lesions caused by subtypes of the Cutaneous Human Papilloma Virus (HPV) family. HPV types are a subset of the large group of the DNA papillomavirus family that are capable of infecting humans and causing cutaneous lesions. HPV's are ubiquitous in the environment and infection occurs most commonly as a result of direct contact with individuals who harbor the virus either clinically (evidence lesions) or subclinically, indirectly through exposure to contaminated surfaces, or by autoinoculation of virus from individual lesions to adjacent uninfected skin. Cutaneous manifestations of HPV infection include common warts (verruca vulgaris), palmar and plantar warts, mosaic warts, flat warts, butcher's warts, and others. Subtypes of the HPV family are also etiologic of oropharyngeal, anogenital, laryngeal warts, papillomas, dysplasias (e.g., CIN (cervical intraepithelial neoplasia) carcinoma in situ, carcinomas, and in the skin lesions seen in epidermodysplasia verruciformis. Common warts are typically hyperkeratotic, exophytic dome-shaped papules or nodules (typically associated with HPV types 1, 2 or 4) and are most commonly located on the fingers (including periungual and subungual regions), dorsal surfaces of the hands, sites prone to trauma (e.g., knees, elbows), but may occur at virtually any other anatomical location.

Condyloma acuminata, more commonly known as genital warts, are typically related to HPV types 6 and 11, 16, 18, and numerous other subtypes (e.g., 33, 35, 39, 40, 43, 45, 51-56, 58 and others), and multiple subtypes may exist in a single lesion. Condyloma acuminata typically present as solitary to multiple fleshy, soft, verrucoid papules that may be dome-shaped, filiform, fungating, “cauliflower-like”, or form confluent plaques. They are typically located in the anogenital region (e.g., penis, vulva, vagina, cervix, perineum, and perianal regions), and may appear in the oropharynx, larynx and even tracheal mucosa, and rarely other cutaneous locations (e.g., trunk, extremities). The lesions are typically benign, but certain HPV subtypes are associated with a risk of malignant potential (e.g., HPV subtypes 16, 18) and may lead to cutaneous carcinomas or carcinomas-in-situ such as bowenoid papulosis or Buschke-Lowenstein tumor, and cervical dysplasias or neoplasia, e.g., cervical intraepithelial neoplasia (CIN).

In immunocompetent individuals, many common cutaneous lesions associated with HPV infection (e.g., warts and condylomata) spontaneously resolve in less than two years. However, warts can be large and/or cosmetically unsightly (e.g., face, hands), spread to distant anatomical regions by autoinoculation, painful (e.g., traumatized or on soles of feet), and untreated warts provide a significant reservoir of HPV infection in the community.

There are currently no specific antiviral therapies available to treat cutaneous HPV infection. Existing therapies are thus directed towards either the direct physical destruction of the lesions with locally destructive modalities such as cryotherapy, electrosurgery, curettage, laser therapy, application of acids (e.g., salicylic acid, trichloroacetic acid); locally cytotoxic therapies, such as topical podophyllin, cantharidin, or topical or intralesional 5-fluorouracil, or bleomycin; topical immunomodulatory therapy (e.g., topical imiquimod, intralesional candida antigen, topical squaric acid dibutyl ester, oral cimetidine) or surgical lesion removal. Various of these therapies are also available as over-the-counter (OTC) wart therapies in lower concentrations (e.g., topical salicylic acid preparations; home “freezing” kits). While these methods can achieve cure rates in some cases, many require multiple visits to a physician's office, specialized training and the use of expensive equipment; they are painful and may require anesthesia and/or analgesia, and they can be complicated by adverse cosmetic outcomes including scarring at the treatment site, and the typical post-surgical risks of bleeding and infection. No one therapy is consistently effective in all cases and in fact, there is great variability among practitioners in the methods employed using each of these techniques with great variability of the results. Recurrences are common and the use of multiple treatment modalities in combination is often necessary to achieve significant improvement. Thus, there exists a great unmet need in the art for a safe and efficacious topical treatment for the cutaneous lesions associated with HPV infection e.g., warts and condyloma.

Mollusca are virally induced lesions caused by subtypes of the DNA poxvirus family of molluscum contagiosum viruses (MCV). There are four subtypes of MCV, (MCV-1 to 4), with MCV-1 being the most prevalent and MCV-2 being most common in adults. Like HPV's, MCV's are ubiquitous in the environment and infection occurs most commonly as a result of direct contact with individuals who harbor the virus either clinically (evidence lesions) or subclinically, indirectly through exposure to contaminated surfaces, or by autoinoculation of virus from individual lesions to adjacent uninfected skin. The infection is most common in the pediatric population, sexually active adults, and the immunocompromised. Molluscum contagiosum lesions are typically flesh-colored, dome-shaped umbilicated (“dimpled”) papules that may occur singly or in clusters and are typically located on the trunk groin or extremities, though may occur on any area of the skin. Individual lesions may spontaneously resolve in several weeks to several months, however, the natural history of the infection from appearance of the first lesions to resolution of the last lesion may last from six months to five years or more.

There are currently no U.S. Food and Drug Administration approved treatments for molluscum contagiosum. There are currently no specific antiviral therapies available to treat cutaneous MCV infection. Existing therapies are thus directed towards either the direct physical destruction of the lesions with locally destructive modalities such as cryotherapy, electrosurgery, curettage, laser therapy, unroofing the lesion with e.g., a needle (“needle-pricking”), application of acids or caustics (e.g., salicylic acid, potassium hydroxide); locally cytotoxic therapies, such as topical podophyllin, cantharidin; topical immunomodulatory therapy (e.g., topical imiquimod, intralesional candida antigen, nitric acid, oral cimetidine) or surgical lesion removal. While these methods can achieve cure rates in some cases, many require multiple visits to a physician's office, specialized training and the use of expensive equipment; they may be painful and may require anesthesia and/or analgesia, and may be anxiety producing and psychologically traumatic, particularly in the pediatric age group. These treatments may be complicated by adverse cosmetic outcomes including pigmentary changes (both hyperpigmentation and hypopigmentation), scarring at the treatment site, bleeding and infection. No one therapy is consistently effective in all cases and in fact, there is great variability among practitioners in the methods employed using each of these techniques with great variability of the results. The use of multiple treatment modalities including the treatment of underlying topical conditions such as atopic dermatitis, which tends to predispose to molluscum and the spread of the lesions, in combination is often necessary to achieve significant improvement. Thus, there exists a great unmet need in the art for a safe and efficacious (topical) treatment for the cutaneous lesions associated with MCV infection.

Hydrogen peroxide (H₂O₂) is a compound that is ubiquitous in the environment. It is the simplest peroxide and a potent oxidizing agent commonly used in innumerable household goods including chlorine-free bleaches, general-purpose cleaning products, and disinfectants, has been employed as the oxidizing component in hair dyes, and has been used in oral hygiene products and tooth-whitening systems for many years. In industry, it is employed in the treatment of wastewater and, in high concentrations, it is used in bleaching paper, pulp, and textiles. Clinically, in addition to its use as an oral topical agent noted above, hydrogen peroxide is widely employed at low concentrations (e.g., 3%-6%) as a wound irrigant and topical antiseptic/disinfectant, and has been in use medicinally since its introduction into clinical practice by Richardson in 1858.

Hydrogen peroxide is an important oxidizing agent in biological systems. The local deleterious effects of reactive oxygen species on the skin are mitigated by the presence of a complex antioxidant defense system that includes, enzymes such as catalase, glutathione peroxidase, superoxide dismutase, thioredoxin reductase, lipoamine, lipid peroxidase and others, as well as non-enzymatic components including ascorbic acid, urates and uric acid, tocopherol, glutathione, ubiquinones, ubiquinol and other water soluble groups. The local application of supra-physiologic concentrations of hydrogen peroxide may overwhelm the antioxidant defense systems in the skin, allowing hydrogen peroxide to act not only through its direct oxidation of organic tissues, generation of reactive oxygen species, and local lipid peroxidation, but also by the generation of local concentrations of oxygen that are toxic to the abnormal lesional (e.g., wart, condyloma acuminatum, molluscum contagiosum) cells.

It has been unexpectedly observed that common wart (verruca vulgaris) lesions evidenced a clinical response after the application of the compositions of embodiments herein. In certain cases, improvement, reduction, or clearance of the cutaneous lesion was observed after one single treatment. In other cases, e.g., with persistent, recurrent, treatment resistant, or with thicker lesions or larger lesions, two or more treatments may be required for a clinical response. In certain cases, improvement or clearance of the cutaneous lesions was observed after a series of treatments (e.g. once weekly treatments). As exemplary benefits of this treatment method, the clinical response was well-tolerated and was brought about without the need for analgesia, without inducing pain, and without inducing the significant adverse events and adverse cosmetic outcomes commonly resulting from other therapies such as, e.g., pigmentary changes (such as hypopigmentation or hyperpigmentation), scarring at the treatment site, bleeding or infection.

Embodiments herein generally are directed to compositions comprising hydrogen peroxide. In some embodiments, the composition may further comprise a surface tension modifying agent. In some embodiments, the surface tension modifying agent is an agent stable in compositions comprising concentrations of hydrogen peroxide disclosed in embodiments herein. In some embodiments, the surface tension modifying agent is in a quantity sufficient to enhance the therapeutic efficacy of the composition. In some embodiments, the 2-propanol is in an amount sufficient to increase the penetration of the composition into the targeted lesion. In some embodiments, the 2-propanol is in an amount sufficient to increase the permeation of the composition into the targeted lesion. In some embodiments, the surface tension modifying agent is in a quantity sufficient to enhance the therapeutic efficacy of the composition while maintaining stability of the composition. In some embodiments, the surface tension modifying agent is in a quantity sufficient to enhance the therapeutic efficacy of the composition while maintaining stability of the composition sufficient for use as a commercially viable formulation. In some embodiments, the surface tension modifying agent is an alcohol. Embodiments herein are also directed to compositions comprising hydrogen peroxide and an alcohol. In some embodiments, the hydrogen peroxide may be a standard grade, food grade, chemical synthesis grade, semiconductor grade, high-test hydrogen peroxide grade, antimicrobial grade, drinking water grade, pharmaceutical grade or cosmetic grade hydrogen peroxide. In some embodiments, the alcohol may be selected from a primary alcohol, a secondary alcohol, a tertiary alcohol, or a combination thereof. In some embodiments, the alcohol may be selected from, but is not limited to, a low molecular weight alcohol, such as methanol, ethanol, butanol, 1-propanol, pentanol, hexanol, octanol, nonanol, decanol, 2-butanol, 2-propanol, 2-pentanol, benzyl alcohol, an isomer thereof, or a combination thereof. In some embodiments, the alcohol is not 1-propanol, ethanol, propanol, butanol, pentanol, hexanol, octanol, nonanol, decanol, 2-butanol, 2-pentanol, or benzyl alcohol. In some embodiments, the alcohol is 2-propanol (also referred to as isopropyl alcohol). In some embodiments, other volatiles such as, for example, acetates such as ethyl and butyl acetate (volatiles used in nail lacquers), cyclomethicone (a volatile silicone which may be included in an emulsifier system) may be used in combination with or in place of an alcohol. Embodiments herein also include a composition consisting essentially of hydrogen peroxide and an alcohol. Some embodiments are directed to a composition consisting of hydrogen peroxide and an alcohol. Some embodiments are directed to a composition comprising hydrogen peroxide and 2-propanol. Some embodiments are directed to a composition consisting essentially of hydrogen peroxide and 2-propanol. Some embodiments are directed to a composition consisting of hydrogen peroxide and 2-propanol.

In some embodiments, the hydrogen peroxide is in an amount of up to about 60% w/w of the composition. In some embodiments, the hydrogen peroxide is in an amount of up to about 55% w/w of the composition. In some embodiments, the hydrogen peroxide is in an amount of up to about 50% w/w of the composition. In some embodiments, the hydrogen peroxide is in an amount of up to about 45% w/w of the composition. In some embodiments, the hydrogen peroxide is in an amount of greater than 40% w/w. In some embodiments, the hydrogen peroxide is in an amount of greater than 40% w/w to about 60% w/w. In some embodiments, the hydrogen peroxide is in an amount of about 45% w/w. In some embodiments, the hydrogen peroxide is in an amount of about 55% w/w. In some embodiments, the hydrogen peroxide is in an amount of about 54% w/w. In some embodiments, the hydrogen peroxide is in an amount of about 59% w/w. In some embodiments, the composition comprises hydrogen peroxide in an amount of about 0.5% w/w to about 99.9% w/w, about 10% w/w to about 99.9% w/w, about 20% w/w to about 99.9% w/w, about 30% w/w to about 99.9% w/w, about 40% w/w to about 99.9% w/w, about 50% w/w to about 99.9% w/w, about 60% w/w to about 99.9% w/w, about 70% w/w to about 99.9% w/w, about 80% w/w to about 99.9% w/w, about 90% w/w to about 99.9% w/w, about 0.5% w/w to about 70% w/w, about 5% w/w to about 70% w/w, about 10% w/w to about 70% w/w, about 15% w/w to about 70% w/w, about 20% w/w to about 70% w/w, about 25% w/w to about 70% w/w, about 30% w/w to about 70% w/w, about 35% w/w to about 70% w/w, about 40% w/w to about 70% w/w, about 40.5% w/w to about 70% w/w, about 41% w/w to about 70% w/w, about 41.5% w/w to about 70% w/w, about 42% w/w to about 70% w/w, about 42.5% w/w to about 70% w/w, about 43% w/w to about 70% w/w, about 43.5% w/w to about 70% w/w, about 44% w/w to about 70% w/w, about 44.5% w/w to about 70% w/w, about 45% w/w to about 70% w/w, about 50% w/w to about 70%6 w/w, about 55% w/w to about 70% w/w, about 60% w/w to about 70% w/w, about 65% w/w to about 70% w/w, up to about 60% w/w, about 0.5% w/w to about 60% w/w, about 5% w/w to about 60% w/w, about 10% w/w to about 60%6 w/w, about 15% w/w to about 60% w/w, about 20% w/w to about 60% w/w, about 23.5% w/w to about 60% w/w, about 25% w/w to about 60% w/w, about 30% w/w to about 60% w/w, about 35% w/w to about 60% w/w, about 40% w/w to about 60% w/w, about 40.5% w/w to about 60% w/w, about 41% w/w to about 60% w/w, about 41.5% w/w to about 60% w/w, about 42% w/w to about 60% w/w, about 42.5% w/w to about 60% w/w, about 43% w/w to about 60% w/w, about 43.5% w/w to about 60% w/w, about 44% w/w to about 60, w/w, about 44.5% w/w to about 60% w/w, about 45% w/w to about 60% w/w, up to about 50% w/w, about 0.5% w/w to about 50% w/w, about 5% w/w to about 50% w/w, about 10% w/w to about 50% w/w, about 15% w/w to about 50% w/w, about 20% w/w to about 50% w/w, about 23.5% w/w to about 50% w/w, about 25% w/w to about 50% w/w, about 30% w/w to about 50% w/w, about 35% w/w to about 50% w/w, about 40% w/w to about 50% w/w, about 40.5% w/w to about 50% w/w, about 41% w/w to about 50% w/w, about 41.5% w/w to about 50% w/w, about 42% w/w to about 50% w/w, about 42.5% w/w to about 50% w/w, about 43% w/w to about 50, w/w, about 43.5% w/w to about 50% w/w, about 44% w/w to about 50% w/w, about 44.5% w/w to about 50% w/w, or about 45% w/w to about 50% w/w. In some embodiments, the hydrogen peroxide may be in an amount of about 0.5% w/w, about 5% w/w, about 10% w/w, about 15%, w/w, about 20% w/w, about 23.5% w/w, about 25% w/w, about 30% w/w, about 32.5% w/w, about 35% w/w, about 40% w/w, about 40.5% w/w, about 41% w/w, about 41.5% w/w, about 42% w/w, about 42.5% w/w, about 43% w/w, about 43.5% w/w, about 44% w/w, about 44.5%6 w/w, about 45% w/w, about 46% w/w, about 47% w/w, about 48, w/w, about 49% w/w, about 50% w/w, about 51% w/w, about 52% w/w, about 53%6 w/v, about 54% w/w, about 55% w/w, about 56% w/w, about 57% w/w, about 58% w/w, about 59% w/w, about 60% w/w, about 65% w/w, about 70% w/w, or a range of any two of these values.

In some embodiments, the hydrogen peroxide may be a stabilized hydrogen peroxide. In some embodiments, the hydrogen peroxide may be a standard grade, food grade, chemical synthesis grade, semiconductor grade, high-test hydrogen peroxide grade, antimicrobial grade, drinking water grade, pharmaceutical grade, active pharmaceutical grade ingredient (API) grade, or cosmetic grade hydrogen peroxide. In some embodiments, the hydrogen peroxide may be a stabilized pharmaceutical grade hydrogen peroxide. In some embodiments, the hydrogen peroxide may be active pharmaceutical ingredient (API) grade hydrogen peroxide. In some embodiments, the hydrogen peroxide is FMC/PeroxyChem 50% w/w “API grade” hydrogen peroxide. In some embodiments, the hydrogen peroxide is FMC/PeroxyChem 59% w/w “API grade” hydrogen peroxide. In some embodiments, the hydrogen peroxide is FMC/PeroxyChem 70% w/w “API grade” hydrogen peroxide. In some embodiments, the hydrogen peroxide may be a stabilized cosmetic grade hydrogen peroxide. In some embodiments, the hydrogen peroxide is FMC/PeroxyChem “Super D” 50% w/w cosmetic grade hydrogen peroxide. In some embodiments, the hydrogen peroxide is FMC/PeroxyChem's “High-Test Hydrogen Peroxide,” which includes stabilized 50% w/w, 70% w/w, and 90% w/w hydrogen peroxide. In some embodiments, the hydrogen peroxide is Arkema Peroxal 50 CG. In some embodiments, the starting hydrogen peroxide concentration is at a concentration sufficient to be diluted to a concentration of about 23% w/w hydrogen peroxide or above in the compositions described herein. In some embodiments, the stabilized hydrogen peroxide has stabilizers in a concentration sufficient to prevent the breakdown/degradation of the hydrogen peroxide when it is diluted to a concentration of about 23% w/w hydrogen peroxide or above in the compositions described herein. In some embodiments, the stabilized hydrogen peroxide has stabilizers in a concentration sufficient to prevent the breakdown/degradation of the surface-tension modifying agent (e.g., alcohol such as 2-propanol) when it is diluted to a concentration of about 23% w/w hydrogen peroxide or above in the compositions described herein. In some embodiments, the stabilized hydrogen peroxide has stabilizers in a concentration sufficient to ensure the stability of the composition to be packaged in an appropriate packaging system, container, or applicator, and to be suitable for commercial use as contemplated in embodiments described herein. For example, in some embodiments, a method of making the composition may comprise a step or steps that the stabilized hydrogen peroxide be diluted down to, e.g., 59% w/w, 55% w/w, 54% w/w, 50% w/w, 45% w/w, or 40% w/w or 32.5% w/w, or 25% w/w, in the final composition. In this scenario, the starting hydrogen peroxide formulation should have a sufficiently high concentration to be able to be diluted to, e.g., 59% w/w, 55% w/w, 54% w/w, 50% w/w, 45% w/w, or 40% w/w, or 32.5% w/w, or 25% w/w, with the addition of water such as deionized water and an additional excipient or excipients such as a surface-tension modifying agent such as an alcohol (e.g., 2-propanol, or the like), as described in embodiments herein, such that it is sufficiently stabilized in order to guarantee a shelf-life suitable to produce a commercially viable formulation.

Hydrogen peroxide is a compound which is highly susceptible to decomposition by the presence of dissolved impurities, mostly transition metal cations and mixtures based on hydrogen peroxide may be unstable, with the hydrogen peroxide concentration diminishing over time due to catalytic decomposition. Impurities causing hydrogen peroxide decomposition are typically contained in water used to dilute the aqueous hydrogen peroxide stock formulation to a desired concentration or in the additional excipients added to the formulation (e.g., 2-propanol). A variety of factors may influence the stability of hydrogen peroxide in solutions, including, for example, the temperature, the concentration of hydrogen peroxide, the pH value, and the presence of impurities having a decomposing effect. To limit the influence of such decomposing factors on stability, it has been discovered that, in some embodiments, a stabilized composition having commercial value for the treatment of the multiple skin conditions described herein, e.g., warts, condyloma acuminatum, molluscum contagiosum, may be achieved by the careful selection of a concentration of 2-propanol and a stabilized (e.g., cosmetic grade) of high concentration (e.g., 50%) aqueous hydrogen peroxide solution. Accordingly, in some embodiments, the composition may comprise a stabilizer or combination of stabilizers. Hydrogen peroxide products from different sources may differ because of a proprietary blend of stabilizers unique to each company and to each product line within each company, and may importantly affect the stability and performance of the final product. Stabilizer levels for each individual stabilizer may vary from above 0 mg/ml to several thousand mg/I each depending on the grade of the hydrogen peroxide, the concentration of the peroxide, and the choice of stabilizers used. In some embodiments, the hydrogen peroxide is supplied by FMC Industrial Chemicals® (now PeroxyChem, LLC.). Sigma Corporation®, Arkema Incorporated®, or the like. In some embodiments, the hydrogen peroxide is supplied by PeroxyChem, LLC. In some embodiments, the hydrogen peroxide is FMC/PeroxyChem “Super D” 50% w/w Cosmetic Grade hydrogen peroxide. Common stabilizers included in hydrogen peroxide formulations may include a stannate (e.g., colloidal stannate, sodium stannate), sodium pyrophosphate, organophosphonates, nitrate, phosphoric acid, colloidal silicate, any other stabilizer known in the art, or a combination thereof. In some embodiments, each stabilizer may be in a concentration of above 0 ppm to about 5000 ppm. In some embodiments, each stabilizer may be in a concentration of above 0 ppm to about 3000 ppm. In some embodiments, each stabilizer may be in a concentration of about 70 ppm to about 5000 ppm. In some embodiments, each stabilizer may be in a concentration of about 70 ppm to about 3000 ppm. In some embodiments, each stabilizer may be in a concentration of about 70 ppm to about 2700 ppm. In some embodiments, each stabilizer may be in a concentration of about 270 ppm to about 5000 ppm. In some embodiments, each stabilizer may be in a concentration of about 300 ppm to about 5000 ppm. In some embodiments, each stabilizer may be in a concentration of about 270 ppm to about 3000 ppm. In some embodiments, each stabilizer may be in a concentration of about 300 ppm to about 3000 ppm. In some embodiments, each stabilizer may be in a concentration of about 300 ppm to about 2700 ppm. In some embodiments, each stabilizer may be in a concentration of about 270 ppm to about 2700 ppm.

The hydrogen peroxide in compositions of embodiments herein may be replaced with or combined with other peroxides. Other peroxides may include, but are not limited to, sodium peroxide, potassium peroxide and potassium superoxide, lithium peroxide, barium peroxide, calcium peroxide, magnesium peroxide, zinc peroxide, tert-butyl hydroperoxide, peracetic acid, dibenzyl peroxide, benzoyl peroxide, lauroyl peroxide, or a combination thereof.

Water, organic solvents such as alcohols, surfactants, and other agents may alter the surface tension of compositions, formulations, and most particularly of solutions. However, little is known about the effects of low concentrations of alcohols or other volatiles on the wettability of normal skin when alcohol/water mixtures are applied, and there have been no reports on the effects of different concentrations of alcohols or other volatiles when incorporated into formulations comprising hydrogen peroxide on the wettability of normal skin or of the wettability of abnormal or lesional skin—such as skin that has been affected by warts, condyloma accuminatum, molluscum contagiosum, or other virally induced or non-virally induced cutaneous growths or lesions. Without being bound by theory, it is believed that the inclusion of alcohol in a stabilized peroxide solution may serve several important functions: The incorporation of a low concentration of the alcohol, e.g., less than 15% w/w 2-propanol, may allow for the incorporation of a therapeutically effective concentration of hydrogen peroxide (e.g., 25% w/w to 60% w/w hydrogen peroxide), where the hydrogen peroxide may be in sufficient concentration to achieve the desired therapeutic effect on the cutaneous lesions, and the alcohol is in an amount sufficient to decrease the surface tension of the formulation and to increase the wettability of the surface of the skin lesion to allow spread of the formulation over the surface and into the surface irregularities of the lesion. In some embodiments, the alcohol is in an amount sufficient to increase the penetration of the composition into the targeted lesion. However, secondary alcohols (e.g., isopropanol (IPA)) are expected to be inherently less stable in high concentrations of hydrogen peroxide than primary alcohols (see below), that is, 2-propanol is expected to be more readily oxidized in high concentrations of hydrogen peroxide than is 1-propanol, and the incorporation of low concentrations of a secondary alcohol (IPA) in a concentration sufficient to fulfill the above requirements (decrease surface tension/increase wettability of the skin lesion/maintain or enhance therapeutic efficacy and increase penetration of the composition into the targeted lesion) and allow the creation of a stable, commercially viable formulation was challenging.

It was surprisingly discovered that 2-propanol, in an amount that may be sufficient to decrease the surface tension of the formulation, may increase the wettability of the surface of the skin lesions, and may maintain or enhance the therapeutic effect of the formulation on the conditions that are the subject of this application, may be stably incorporated into a highly concentrated hydrogen peroxide solution. As noted above, isopropanol, a secondary alcohol, is expected to be more easily oxidized than 1-propanol, the primary alcohol in the presence of high concentrations of hydrogen peroxide. Without intending to be bound by theory, the mechanism of the oxidation of alcohols in high concentrations of hydrogen peroxide is first, the generation of hydroxyl radicals from the decomposition of the hydrogen peroxide. This process can be accelerated by the presence of catalytic metals or other catalysts such as those that might be introduced by the addition of excipients and/or impurities (mainly transition metal cations). The hydroxyl radical would then abstract a hydrogen atom from the carbon adjacent to the oxygen on the alcohol molecule, resulting in a carbon radical. In the case of isopropanol, this is a secondary radical which is stabilized by the electrons on the oxygen and both methyl groups next to the carbon. For 1-propanol, this is a primary radical with just one alkyl group next to it, which is less stable and more difficult to form. This intermediate would lose the hydrogen atom of the hydroxyl group to form ketone or aldehyde and the aldehyde, propanal from 1-propanol, can be further oxidized to propionic acid. In preparations of hydrogen peroxides that are of high purity and are highly stabilized or have high concentrations of stabilizers (and with lower concentrations of catalyst/impurities), the decomposition of peroxide may be slowed and thus the decomposition of the included alcohol may be slowed. Though without an obvious catalyst, the decomposition of peroxide is slow. The apparent reaction rate might be complex equation involving any trace catalyst (e.g., catalytic metal), peroxide and alcohol concentrations, but considering the alcohol alone, a secondary alcohol is more easily oxidized than the primary one. It was surprisingly discovered, however, that by employing a stabilized formulation of hydrogen peroxide (e.g., FMC/PeroxyChem “Super D”), the incorporation of low concentrations of the secondary alcohol 2-propanol (IPA) into a stable, commercially viable formulation, was indeed possible. Additionally and importantly, primary alcohols, such as 1-propanol are known to provoke cutaneous erythema (redness) and irritation of the skin and may produce a “flushing reaction” when applied to the skin due to the generation of aldehyde intermediates of primary alcohol breakdown. Alcohol dehydrogenase (ADH), which is present in the skin, acts on and breaks down primary alcohols, such as 1-propanol, but does not act on secondary alcohols, such as 2-propanol. Thus, only primary alcohols, which can be oxidized to the corresponding aldehydes by alcohol dehydrogenase (ADH) present in the skin, and not secondary (or tertiary) alcohols, elicit this cutaneous erythema reaction by this important mechanism. By incorporating a secondary alcohol such as 2-propanol (rather than a primary alcohol such as 1-propanol) into the composition, adverse cutaneous erythematous reactions resulting from or exacerbated by ADH catalyzed aldehyde intermediates may be avoided or mitigated.

It was thus surprisingly discovered that the addition of lower concentrations of alcohol, such as those described in embodiments herein, and particularly 2-propanol, to hydrogen peroxide increased the wettability of the skin lesion. Additionally, skin lesions, by nature, may have crevices and/or invaginations or surface irregularities that may make penetration of the hydrogen peroxide difficult. Accordingly, in some embodiments, a composition may comprise a hydrogen peroxide and a surface tension modifying agent. In some embodiments, the surface tension modifying agent may be an alcohol. The alcohol may be in an amount sufficient to decrease the surface tension of the composition to a level that effectively increases the penetration of the composition into such crevices and/or invaginations of the skin lesion, increase the surface area of reaction and increase the therapeutic efficacy and/or clinical response of the skin lesion to the therapeutic composition. In some embodiments, the composition further comprises another surface-tension modifying agent. In some embodiments, the surface tension modifying agent may be selected from, without limitation, a surfactant, e.g., an anionic or nonionic surfactant, a water-soluble surfactant such as a polysorbate, SLS (sodium lauryl sulfate), polypropylene glycol (PPG) stearate such as Arlamol, PEG (polyethylene glycol) stearate, steareth, ceteareth, polyoxyl stearate, or the like, or a combination thereof. The surface tension modifying agent may be in an amount sufficient to decrease the surface tension of the composition to a level that effectively increases the penetration of the composition into the crevices, invaginations and/or surface irregularities of the skin lesion, increases the surface area of reaction, and increases the therapeutic efficacy and/or clinical response of the skin lesion to the therapeutic composition, or a combination thereof.

In particular, it was surprisingly discovered that 2-propanol is a particularly effective alcohol for incorporation in the compositions described herein. In fact, it was unexpectedly discovered that 2-propoanol is a more suitable and effective alcohol in the compositions described herein than 1-propanol. 1-propanol does allow for release of hydrogen peroxide, may reduce the surface tension of the composition to increase penetration of the composition into the skin, and was expected to be more stable (i.e. less likely to be oxidized) in formulations comprising high concentrations of hydrogen peroxide. In fact, when compared to 2-propanol, 1-propanol does, in some concentrations, provide increased release or increased rate of release of hydrogen peroxide and may reduce surface tension more (on a weight to weight basis). However, it was surprisingly discovered that despite its seemingly potential desirable effects, 1-propanol is actually a less effective alcohol than 2-propoanol in compositions of embodiments disclosed herein. Without wishing to be bound by theory, it is believed that (i) though there is a trend towards 1-propanol containing compositions to release more hydrogen peroxide where 1-propanol is in higher concentrations, 2-propanol containing compositions effectively release hydrogen peroxide and exhibit a more constant rate of release across the desired concentrations in the embodiments described herein; (ii) though 1-propanol may reduce the surface tension of the hydrogen peroxide formulation more, on a weight for weight basis, than 2-propanol, the surface tension reduction induced in the compositions of the preferred embodiments by 1-propanol is excessive and suboptimal as it may induce such a great reduction that the composition would undesirably spread off the target lesion or area and onto surrounding non-lesional skin, leading to adverse effects such as irritation and erythema due at least in part to the generation of undesirable aldehyde intermediates as discussed above; and (iii) though theoretically, as discussed, the primary alcohol, 1-propanol, would be expected to exhibit greater stability in high concentration hydrogen peroxide formulations than the secondary alcohol (i.e. 2-propanol), 1-propanol appears to be oxidized to a greater degree by hydrogen peroxide in the high concentration hydrogen peroxide formulations of the preferred embodiments than is 2-propanol, and is, in fact, less stable. Thus, the incorporation of 2-propanol in compositions described herein provides for significant advantages over the incorporation of 1-propanol, including, but not limited to, providing for a therapeutically effective formulation which is more stable—leading to improved clinical efficacy of the composition, lower tendency to spread away from the intended site of application and, therefore, a more favorable safety profile

The amount of alcohol in the composition may also be limited by the peroxide concentration in the formulation. For example, if a high concentration of peroxide is desired, the concentration of alcohol may necessarily be lowered in order to maintain the high concentration of peroxide in the formulation. In some embodiments, the alcohol may be in an amount sufficient to decrease the surface tension of the composition, to increase the penetration of the composition into the crevices and/or invaginations of the skin lesion, increase the surface area of reaction and increase the therapeutic efficacy and/or clinical response of the skin lesion to the therapeutic composition. In some embodiments, the formulation does not spread undesirably onto the surrounding non-lesional unaffected skin, which may occur with too large an amount of alcohol or too great a reduction in surface tension. In some embodiments the formulation does not irritate the surrounding, non-lesional, unaffected skin. In some embodiments, the formulation does not cause erythema to surrounding non-lesional unaffected skin. Erythema and irritation of the surrounding, non-lesional, unaffected skin may be caused by generation of irritating, erythema inducing intermediates, such as aldehydes, which may be due to the breakdown of suboptimal alcohols in the composition (e.g., primary alcohols, such as 1-propanol).

In some embodiments, the composition comprises a surface tension modifying agent. In some embodiments, the surface tension modifying agent is an alcohol. In some embodiments, the surface tension modifying agent is in an amount sufficient to decrease the surface tension of the hydrogen peroxide and water formulation. In some embodiments, the composition comprises an alcohol in an amount sufficient to decrease the surface tension of the hydrogen peroxide and water formulation. In some embodiments, the alcohol is a primary alcohol, a secondary alcohol, a tertiary alcohol, or a combination thereof. In some embodiments, the secondary alcohol is 2-propanol. Without alcohol, such as 2-propanol, to decrease the surface tension, the hydrogen peroxide-water formulation may sit on the surface of the lesion, without penetrating the lesion and/or the surface imperfections, irregularities, crevices of the lesion. In some embodiments, the surface tension modifying agent may be in an amount sufficient to decrease the surface tension of the composition to a level that effectively increases the penetration of the composition into such crevices and/or invaginations of the skin lesion, increase the surface area of reaction, increase the therapeutic efficacy and/or clinical response of the skin lesion to the therapeutic composition while minimizing irritation of the surrounding skin, or any combination thereof. Furthermore, alcohols that lower the surface tension of hydrogen peroxide composition excessively may run the risk of easily spreading off the application site and across non-lesional skin, causing less activity at the needed site and unwanted irritation and other adverse effects on the surrounding, unaffected skin.

In some embodiments, the alcohol in compositions of embodiments herein may be replaced with other volatile agents. Such volatile agents may include, but are not limited to, volatiles such as acetates, e.g., ethyl acetate and butyl acetate (volatiles used in nail lacquers), cyclomethicone (a volatile silicone which may be included in an emulsifier system), and various other volatiles in addition to those shown and described herein, which will become apparent to those skilled in the art from the foregoing description. Such additional volatile agents may be used in combination with or in place of an alcohol.

In some embodiments, the alcohol may be selected from a primary alcohol, a secondary alcohol, a tertiary alcohol, or a combination thereof. In some embodiments, the alcohol may include methanol, ethanol, butanol, 1-propanol, pentanol, hexanol, octanol, nonanol, decanol, 2-butanol, 2-propanol, 2-pentanol, benzyl alcohol, an isomer thereof or a combination thereof. In some embodiments, the alcohol is 2-propanol. Though embodiments herein may refer to 2-propanol in particular, one skilled in the art would understand that other alcohols and/or volatiles, such as, but not limited to, those described above may be used in place of 2-propanol in such embodiments.

2-propanol (also referred to as isopropyl alcohol or isopropanol) is a chemical compound with the molecular formula C₃H₈O or C₃H₇OH. It is a colorless, flammable chemical compound with a strong odor. It is the simplest example of a secondary alcohol, where the alcohol carbon atom is attached to two other carbon atoms sometimes shown as (CH₃)₂CHOH. It is a structural isomer of propanol. 2-propanol is miscible in water, alcohol, ether and chloroform. It will dissolve ethyl cellulose, polyvinyl butyral, many oils, alkaloids, gums and natural resins. It is insoluble in salt solutions. Unlike ethanol or methanol, 2-propanol may be separated from aqueous solutions by adding a salt such as sodium chloride, sodium sulfate, or any of several other inorganic salts, since the alcohol is much less soluble in saline solutions than in salt-free water. 2-propanol has many medical and pharmaceutical uses and is typically used topically in concentrations of about 60% to about 70% in water as a topical disinfectant and in concentrations of about 60% to about 75% v/v solution in gels as a hand sanitizer. 2-propanol is also used as a water-drying aid for the treatment/prevention of otitis externa (swimmer's ear) in a concentration of up to 95% w/w.

In some embodiments, the alcohol is in an amount of up to about 0.1% w/w, up to about 0.25% w/w, up to about 0.5% w/w, up to about 1% w/w, up to about 2% w/w, up to about 2.5% w/w, up to about 3% w/w, up to about 4% w/w, up to about 5% w/w, up to about 8% w/w, up to about 10% w/w, up to about 14% w/w, up to about 15% w/w, up to about 20% w/w, or up to about 25% w/w of the composition. The use of low concentrations of alcohol, e.g., 2-propanol, as described in embodiments herein, allows for the use of stabilized hydrogen peroxides of therapeutically high concentrations (such as about 23% or greater) of hydrogen peroxide, such that the stabilizers in the hydrogen peroxide are able to maintain the chemical stability of the formulation without being affected by the alcohol (and its impurities). In some embodiments, the alcohol may comprise up to about 25% w/w of the composition. In some embodiments, the alcohol may be in an amount of about 0.05% w/w to about 25% w/w, about 0.5% w/w to about 25% w/w, about 1% w/w to about 25% w/w, about 2.5% w/w to about 25% w/w, about 5% w/w to about 25% w/w, about 10% w/w to about 25% w/w, about 15% w/w to about 25% w/w, or about 20% w/w to about 25% w/w, about 0.05% w/w to about 15% w/w, about 0.5% w/w to about 25% w/w, up to about 5% w/w, about 0.01% w/w to about 5% w/w, about 0.1% w/w to about 5% w/w, about 0.5% w/w to about 5% w/w, about 1% w/w to about 5% w/w, about 1.5% w/w to about 5% w/w, about 2% w/w to about 5% w/w, about 2.5% w/w to about 5% w/w, about 3% w/w to about 5% w/w, about 3.5% w/w, to about 5% w/w, about 4% w/w to about 5% w/w, about 4.5% w/w to about 5% w/w, or the like. In some embodiments, the alcohol may be in an amount of about 0.01% w/w, about 0.10% w/w, about 0.25% w/w, 0.5% w/w, about 0.75% w/w, about 1% w/w, about 2% w/w, about 2.5% w/w, about 3% w/w, about 4% w/w about 5% w/w, about 10% w/w, about 15% w/w, about 20% w/w, about 25% w/w, or a range of any two of these values.

In some embodiments, the composition comprises greater than 40% w/w hydrogen peroxide and up to about 10% w/w alcohol. In some embodiments, the composition comprises up to about 70% w/w hydrogen peroxide and up to about 10% w/w alcohol. In some embodiments, the composition comprises up to about 60% w/w hydrogen peroxide and up to about 10% w/w alcohol. In some embodiments, the composition comprises greater than 40% w/w to about 60% w/w hydrogen peroxide and up to about 10% w/w alcohol. In some embodiments, the composition comprises up to about 50% w/w hydrogen peroxide and up to about 10% w/w alcohol. In some embodiments, the composition comprises greater than 40% w/w to about 50% w/w hydrogen peroxide and up to about 10% w/w alcohol. In some embodiments, the composition comprises about 40% w/w hydrogen peroxide and about 10% w/w alcohol. In some embodiments, the composition comprises about 41% w/w hydrogen peroxide and up to about 10% w/w alcohol. In some embodiments, the composition comprises about 42% w/w hydrogen peroxide and up to about 10% w/w alcohol. In some embodiments, the composition comprises about 43% w/w hydrogen peroxide and up to about 10% w/w alcohol. In some embodiments, the composition comprises about 44% w/w hydrogen peroxide and up to about 10% w/w alcohol. In some embodiments, the composition comprises about 45% w/w hydrogen peroxide and up to about 10% w/w alcohol. In some embodiments, the composition comprises about 46% w/w hydrogen peroxide and up to about 10% w/w alcohol. In some embodiments, the composition comprises about 47% w/w hydrogen peroxide and up to about 10% w/w alcohol. In some embodiments, the composition comprises about 48% w/w hydrogen peroxide and up to about 10% w/w alcohol. In some embodiments, the composition comprises about 49% w/w hydrogen peroxide and up to about 10% w/w alcohol. In some embodiments, the composition comprises up to about 50% w/w hydrogen peroxide and 10% why alcohol. In some embodiments, the composition comprises up to about 50% w/w hydrogen peroxide and up to about 5% w/w alcohol. In some embodiments, the composition comprises up to about 60% w/w hydrogen peroxide and 10% w/w alcohol. In some embodiments, the composition comprises up to about 60% w/w hydrogen peroxide and up to about 5% w/w alcohol. In some embodiments, the composition comprises up to about 55% w/w hydrogen peroxide and 10% w/w alcohol. In some embodiments, the composition comprises up to about 55% w/w hydrogen peroxide and up to about 5% w/w alcohol. In some embodiments, the composition comprises greater than 40% w/w hydrogen peroxide and up to about 5% w/w alcohol. In some embodiments, the composition comprises greater than 40% w/w to about 60% w/w hydrogen peroxide and up to about 5% w/w alcohol. In some embodiments, the composition comprises about 40% w/w hydrogen peroxide and about 5% w/w alcohol. In some embodiments, the composition comprises about 41% w/w hydrogen peroxide and up to about 5% w/w alcohol. In some embodiments, the composition comprises about 42% w/w hydrogen peroxide and up to about 5% w/w alcohol. In some embodiments, the composition comprises about 43% w/w hydrogen peroxide and up to about 5% w/w alcohol. In some embodiments, the composition comprises about 44% w/w hydrogen peroxide and up to about 5% w/w alcohol. In some embodiments, the composition comprises about 45% w/w hydrogen peroxide and up to about 5% w/w alcohol. In some embodiments, the composition comprises about 46% w/w hydrogen peroxide and up to about 5% w/w alcohol. In some embodiments, the composition comprises about 47% w/w hydrogen peroxide and up to about 5% w/w alcohol. In some embodiments, the composition comprises about 48% w/w hydrogen peroxide and up to about 5% w/w alcohol. In some embodiments, the composition comprises about 49% w/w hydrogen peroxide and up to about 5% w/w alcohol. In some embodiments, the composition comprises about 50% w/w hydrogen peroxide and 5% w/w alcohol. In some embodiments, the composition comprises about 60% w/w hydrogen peroxide and 5% w/w alcohol. In some embodiments, the composition comprises up to about 60% w/w hydrogen peroxide and up to about 2.5% w/w alcohol. In some embodiments, the composition comprises about 50% w/w hydrogen peroxide and up to about 2.5% w/w alcohol. In some embodiments, the composition comprises greater than 40% w/w hydrogen peroxide and up to about 2.5% w/w alcohol. In some embodiments, the composition comprises greater than 40% w/w to about 60% w/w hydrogen peroxide and up to about 2.5% w/w alcohol. In some embodiments, the composition comprises about 40% w/w hydrogen peroxide and about 2.5% w/w alcohol. In some embodiments, the composition comprises about 41% w/w hydrogen peroxide and up to about 2.5% w/w alcohol. In some embodiments, the composition comprises about 42% w/w hydrogen peroxide and up to about 2.5% w/w alcohol. In some embodiments, the composition comprises about 43% w/w hydrogen peroxide and up to about 2.5% w/w alcohol. In some embodiments, the composition comprises about 44% w/w hydrogen peroxide and up to about 2.5% w/w alcohol. In some embodiments, the composition comprises about 45% w/w hydrogen peroxide and up to about 2.5% w/w alcohol. In some embodiments, the composition comprises about 46% w/w hydrogen peroxide and up to about 2.5% w/w alcohol. In some embodiments, the composition comprises about 47% w/w hydrogen peroxide and up to about 2.5% w/w alcohol. In some embodiments, the composition comprises about 48% w/w hydrogen peroxide and up to about 2.5% w/w alcohol. In some embodiments, the composition comprises about 49% w/w hydrogen peroxide and up to about 2.5% w/w alcohol. In some embodiments, the composition comprises up to about 50% w/w hydrogen peroxide and 2.5% w/w alcohol. In some embodiments, the composition comprises about 60% w/w hydrogen peroxide and 2.5% w/w alcohol. In some embodiments, the composition comprises greater than 40% w/w hydrogen peroxide and up to about 2% w/w alcohol. In some embodiments, the composition comprises greater than 40% w/w to about 60% w/w hydrogen peroxide and up to about 2% w/w alcohol. In some embodiments, the composition comprises about 40% w/w hydrogen peroxide and about 2% w/w alcohol. In some embodiments, the composition comprises about 41% w/w hydrogen peroxide and up to about 2% w/w alcohol. In some embodiments, the composition comprises about 42% w/w hydrogen peroxide and up to about 2% w/w alcohol. In some embodiments, the composition comprises about 43% w/w hydrogen peroxide and up to about 2% w/w alcohol. In some embodiments, the composition comprises about 44% w/w hydrogen peroxide and up to about 2% w/w alcohol. In some embodiments, the composition comprises about 45% w/w hydrogen peroxide and up to about 2% w/w alcohol. In some embodiments, the composition comprises about 46% w/w hydrogen peroxide and up to about 2% w/w alcohol. In some embodiments, the composition comprises about 47% w/w hydrogen peroxide and up to about 2% w/w alcohol. In some embodiments, the composition comprises about 48% w/w hydrogen peroxide and up to about 2% w/w alcohol. In some embodiments, the composition comprises about 49% w/w hydrogen peroxide and up to about 2% w/w alcohol. In some embodiments, the composition comprises up to about 50% w/w hydrogen peroxide and 2% w/w alcohol. In some embodiments, the composition comprises about 60% w/w hydrogen peroxide and 2% w/w alcohol. In some embodiments, the composition comprises up to about 60% w/w hydrogen peroxide and 2% w/w alcohol. In some embodiments, the alcohol is 2-propanol.

In some embodiments, the composition consists essentially of up to about 60% w/w hydrogen peroxide. In some embodiments, the composition consists essentially of greater than 40% w/w hydrogen peroxide. In some embodiments, the composition consists essentially of greater than 40% w/w to about 60% w/w hydrogen peroxide. In some embodiments, the composition consists essentially of up to about 50% w/w hydrogen peroxide. In some embodiments, the composition consists essentially of about 59% w/w hydrogen peroxide. In some embodiments, the composition consists essentially of about 55% w/w hydrogen peroxide. In some embodiments, the composition consists essentially of about 54% w/w hydrogen peroxide. In some embodiments, the composition consists essentially of about 50% w/w hydrogen peroxide. In some embodiments, the composition consists essentially of about 45% w/w hydrogen peroxide. In some embodiments, the composition consists essentially of up to about 60% w/w hydrogen peroxide and an alcohol. In some embodiments, the composition consists essentially of greater than 40% w/w hydrogen peroxide and an alcohol. In some embodiments, the composition consists essentially of greater than 40% w/w and up to about 5% w/w alcohol. In some embodiments, the composition consists essentially of greater than 40% w/w to about 60% w/w hydrogen peroxide and up to about 5% w/w alcohol. In some embodiments, the composition consists essentially of about 41% w/w hydrogen peroxide and up to about 5% w/w alcohol. In some embodiments, the composition consists essentially of about 42% w/w hydrogen peroxide and up to about 5% w/w alcohol. In some embodiments, the composition consists essentially of about 43% w/w hydrogen peroxide and up to about 5% w/w alcohol. In some embodiments, the composition consists essentially of about 44% w/w hydrogen peroxide and up to about 5% w/w alcohol. In some embodiments, the composition consists essentially of about 45% w/w hydrogen peroxide and up to about 5% w/w alcohol. In some embodiments, the composition consists essentially of about 46% w/w hydrogen peroxide and up to about 5% w/w alcohol. In some embodiments, the composition consists essentially of about 47% w/w hydrogen peroxide and up to about 5% w/w alcohol. In some embodiments, the composition consists essentially of about 48% w/w hydrogen peroxide and up to about 5% w/w alcohol. In some embodiments, the composition consists essentially of about 49%⁰ w/w hydrogen peroxide and up to about 5% w/w alcohol. In some embodiments, the composition consists essentially of up to about 50% w/w hydrogen peroxide and alcohol. In some embodiments, the composition consists essentially of about 40% w/w hydrogen peroxide and about 5% w/w alcohol. In some embodiments, the composition consists essentially of about 35% w/w hydrogen peroxide and about 5% w/w alcohol. In some embodiments, the composition consists essentially of about 32.5% w/w hydrogen peroxide and about 5% w/w alcohol. In some embodiments, the composition consists essentially of about 25% w/w hydrogen peroxide and about 5% w/w alcohol. In some embodiments, the composition consists essentially of up to about 60% w/w hydrogen peroxide and an alcohol. In some embodiments, the composition consists essentially of about 59% w/w hydrogen peroxide and about 5% w/w alcohol. In some embodiments, the composition consists essentially of about 55% w/w hydrogen peroxide and about 5% w/w alcohol. In some embodiments, the composition consists essentially of about 54% w/w hydrogen peroxide and about 5% w/w alcohol. In some embodiments, the composition consists essentially of about 50% w/w hydrogen peroxide and about 5% w/w alcohol. In some embodiments, the composition consists essentially of about 45% w/w hydrogen peroxide and about 5% w/w alcohol.

In some embodiments, the composition consists of up to about 60% w/w hydrogen peroxide. In some embodiments, the composition consists of up to about 50% w/w hydrogen peroxide. In some embodiments, the composition consists of greater than 40% w/w hydrogen peroxide. In some embodiments, the composition consists of greater than 40% w/w to about 60% w/w hydrogen peroxide. In some embodiments, the composition consists of about 45% w/w hydrogen peroxide. In some embodiments, the composition consists of up to about 60% w/w hydrogen peroxide and an alcohol. In some embodiments, the composition consists of up to about 50% w/w hydrogen peroxide and an alcohol. In some embodiments, the composition consists of greater than 40% w/w hydrogen peroxide and up to about 5% w/w alcohol. In some embodiments, the composition consists of greater than 40% w/w to about 60% w/w hydrogen peroxide and up to about 5% w/w alcohol. In some embodiments, the composition consists of about 41% w/w hydrogen peroxide and up to about 5% w/w alcohol. In some embodiments, the composition consists of about 42% w/w hydrogen peroxide and up to about 5% w/w alcohol. In some embodiments, the composition consists of about 43% w/w hydrogen peroxide and up to about 5% w/w alcohol. In some embodiments, the composition consists of about 44% w/w hydrogen peroxide and up to about 5% w/w alcohol. In some embodiments, the composition consists of about 45% w/w hydrogen peroxide and up to about 5% w/w alcohol. In some embodiments, the composition consists of about 46% w/w hydrogen peroxide and up to about 5% w/w alcohol. In some embodiments, the composition consists of about 47% w/w hydrogen peroxide and up to about 5% w/w alcohol. In some embodiments, the composition consists of about 48% w/w hydrogen peroxide and up to about 5% w/w alcohol. In some embodiments, the composition consists of about 49% w/w hydrogen peroxide and up to about 5% w/w alcohol. In some embodiments, the composition consists of up to about 50% w/w hydrogen peroxide and alcohol. In some embodiments, the composition consists of about 40% w/w hydrogen peroxide and about 5% w/w alcohol. In some embodiments, the composition consists of about 35% w/w hydrogen peroxide and about 5% w/w alcohol. In some embodiments, the composition consists of about 32.5% w/w hydrogen peroxide and about 5% w/w alcohol. In some embodiments, the composition consists of about 25% w/w hydrogen peroxide and about 5% w/w alcohol. In some embodiments, the composition consists of up to about 60% w/w hydrogen peroxide and an alcohol. In some embodiments, the composition consists of about 59% w/w hydrogen peroxide and about 5% w/w alcohol. In some embodiments, the composition consists of about 55% w/w hydrogen peroxide and about 5% w/w alcohol. In some embodiments, the composition consists of about 54% w/w hydrogen peroxide and about 5% why alcohol. In some embodiments, the composition consists of about 50% w/w hydrogen peroxide and about 5% w/w alcohol. In some embodiments, the composition consists of about 45% w/w hydrogen peroxide and about 5% w/w alcohol. In some embodiments, the alcohol is 2-propanol.

In some embodiments, the alcohol decreases the surface tension of the composition. In some embodiments, the alcohol increases the penetration of the hydrogen peroxide into the skin imperfections of the subject, such as the irregularities, crevices and imperfections of the skin or skin lesion. In some embodiments, the alcohol defats the subject's skin or skin lesion of the subject, thereby allowing better penetration of the hydrogen peroxide into the subject's skin or skin lesion. In some embodiments, the alcohol increases the wettability of the surface of the skin, including the wettability of the skin growth or lesion. In some embodiments, the 2-propanol is in an amount sufficient to decrease the surface tension of the composition. In some embodiments, the 2-propanol is in an amount sufficient to decrease the surface tension of the composition sufficient to enhance the penetration of the composition into a targeted skin area or lesion. In some embodiments the surface tension modifying agent is in a quantity sufficient to enhance the therapeutic efficacy of the composition.

In some embodiments, the alcohol increases the effective concentration of the hydrogen peroxide when administered. Hydrogen peroxide is bactericidal, virucidal, sporocidal, and fungicidal, and may be a sterilant at varying concentrations and contact times. In some embodiments, the hydrogen peroxide is in a concentration sufficient to be virucidal. In some embodiments the hydrogen peroxide has sufficient contact time with the skin or skin lesion for it to exhibit its bactericidal, virucidal, sporocidal, fungicidal or sterilant effects. In some embodiments, the hydrogen peroxide has sufficient contact time with the skin or skin lesion sufficient for it to exhibit virucidal effects. Without being bound by theory, the alcohol may increase the effective concentration of the hydrogen peroxide when it evaporates after being administered, and may increase the oxidative and/or germicidal activity of the formulation. Additionally, the increased penetration of the formulation may increase the surface area or contact time of the solution with the skin or skin lesion and lead to enhanced effect as a germicide or sterilant. Additionally, and without being bound by theory, the increased depth of penetration and/or surface area of contact may induce or augment a local or systemic immune response that may supplement the above therapeutic effects and result in more rapid or more effective clearance of not only the target wart lesion being treated, but by other non-target warts located at sites distant from the treated target wart site.

In some embodiments, the composition may be administered topically. In some embodiments, the composition may be a solution. In some embodiments, the composition may be in a gel formulation. In some embodiments, the solution or gel formulation may be in two or more parts to be admixed at or immediately before the time of administration. In some embodiments, the composition may be in a cream, lotion, ointment, foam, transdermal patch, powder, solid, tape, paste or tincture. In some embodiments, the methods of treating described in embodiments herein require only one single application of the composition of embodiments herein. In some embodiments, the methods of treating described in embodiments herein require two or more applications of the composition of embodiments herein. In some embodiments, the methods of treating described in embodiments herein require multiple applications of the composition of embodiments herein.

In some embodiments, the composition may further include a pharmaceutically acceptable excipient. In some embodiments, the composition may further include an emollient, an emulsifier, a gelling agent, an additive, or a combination thereof. In some embodiments, the additive may be selected from preservatives, emulsion stabilizers, pH adjusters, chelating agents, viscosity modifiers, anti-oxidants, surfactants, detergents, emollients, opacifying agents, skin conditioners, buffers, or a combination thereof.

Some embodiments herein are directed to a gel formulation comprising hydrogen peroxide, and a gelling agent. In some embodiments, the gel formulation may further comprise an alcohol. In some embodiments, the gel formulation may further comprise a pharmaceutically acceptable excipient. In some embodiments, the gelling agent may be selected from Carbopol ETD 2020, Carbopol 980 NF, Carbopol 974P, Carbopol Ultrez 10, or the like. In some embodiments, the gelling agent may be high molecular weight, cross linked copolymers of acrylic acid and a hydrophobic comonomer or a copolymer (e.g., Pemulen TR-1); Polycarbophil AA-1; PVP (polyvinyl pyrrolidone); Eudragit; Poloxamer; Sepineo; Bentonite; Aerosil (silicates); Hyaluronic Acids; cross-linked Hyaluronic Acids; a combination thereof, or a compositional or chemical equivalent thereof possessive of the qualities required for the composition of embodiments described herein. In some embodiments, the gel composition is kept in two-parts and mixed at or immediately before the time of application. For example, the hydrogen peroxide and 2-propanol (first part) could be kept separate from the gelling agent (second part) until the time of administration or immediately before. As another example, the hydrogen peroxide, 2-propanol and gelling agent could each be separated into three parts and mixed at or immediately before the time of application. Additional parts may be possible for additional excipients or such excipients may be incorporated into existing parts. At or immediately before the time of administration, the multi-part gel formulation may be mixed and applied topically to the skin as a single gel formulation.

Some embodiments are directed to a gel formulation that may be delivered in an applicator that mixes two or more components of the gel formulation at or immediately before the time of application. In some embodiments, the gel formulation compartment applicator comprises at least one frangible compartment (e.g., gelling agent in the main compartment of the applicator, with the peroxide in a glass ampule within or alongside that compartment). Some exemplary applicators may include syringe-like applicators or “double-barrel” applicators that can freshly mix (e.g., “vortex mix”) two or more components that need to be held in separate compartments for stability reasons, but which can be mixed at or immediately before the time of application.

In some embodiments, the composition further includes a buffer. In some embodiments, the buffer may be selected from triethanolamine, low pH buffers such as sodium acetate, citrate, phosphate, glycine, hydrogen chloride, citrate and phosphate, glycine and hydrogen chloride, the like, or a combination thereof. In some embodiments, the buffer may be present in an amount of about 0.001% w/w to about 15% w/w. In some embodiments, the buffer is present in an amount of about 0.001% w/w, 0.01% w/w, 0.05% w/w, 0.1% w/w, 0.5% w/w, 1% w/w, 5% w/w, 10% w/w, 15% w/w, or a range of any two of these values. In some embodiments the buffer is present in any amount necessary to optimally adjust the pH of the composition.

In some embodiments, the composition has a surface tension of about 15 mN·m-1 to about 80 mN·m-1 at room temperature. In some embodiments, the composition has a surface tension of about 20 mN·m-1 to about 80 mN·m-1, about 30 mN·m-1 to about 80 mN·m-1, about 40 mN·m-1 to about 80 mN·m-1, about 50 mN·m-1 to about 80 mN·m-1, about 35 mN·m-1 to about 80 mN·m-1, about 35 mN·m-1 to about 70 mN·m-1, about 35 mN·m-1 to about 60 mN·m-1, about 35 mN·m-1 to about 50 mN·m-1 at 37° C., about 40 mN·m-1 to about 80 mN·m-1, about 40 mN·m-1 to about 70 mN·m-1, about 40 mN·m-1 to about 60 mN·m-1, about 40 mN·m-1 to about 50 mN·m-1, about 45 mN·m-1 to about 80 mN·m-1, about 45 mN·m-1 to about 70 mN·m-1, about 45 mN·m-1 to about 60 mN·m-1, or about 45 mN·m-1 to about 50 mN·m-1 at room temperature. In some embodiments, the composition has a surface tension of about 15 mN·m-1, about 20 mN·m-1, about 30 mN·m-1, about 36 mN·m-1, about 41 mN·m-1, about 48 mN·m-1, about 54 mN·m-1, about 75 mN·m-1, about 40 mN·m-1, about 50 mN·m-1, about 60 mN·m-1, about 70 mN·m-1, about 80 mN·m-1, or a range of any two of these values at room temperature. In some embodiments, the composition has a surface tension of about 42 mN·m-1 to about 55 mN·m-1 at room temperature. In some embodiments, the composition has a surface tension of about 42 mN·m-1 to about 50 mN·m-1 at room temperature.

In some embodiments, the composition has a surface tension of about 15 mN·m-1 to about 80 mN·m-1 at 37° C. In some embodiments, the composition has a surface tension of about 20 mN·m-1 to about 80 mN·m-1, about 30 mN·m-1 to about 80 mN·m-1, about 40 mN·m-1 to about 80 mN·m-1, about 50 mN·m-1 to about 80 mN·m-1, about 35 mN·m-1 to about 80 mN·m-1, about 35 mN·m-1 to about 70 mN·m-1, about 35 mN·m-1 to about 60 mN·m-1, about 35 mN·m-1 to about 50 mN·m-1 at 37° C., about 40 mN·m-1 to about 80 mN·m-1, about 40 mN·m-1 to about 70 mN·m-1, about 40 mN·m-1 to about 60 mN·m-1, about 40 mN·m-1 to about 50 mN·m-1, about 45 mN·m-1 to about 80 mN·m-1, about 45 mN·m-1 to about 70 mN·m-1, about 45 mN·m-1 to about 60 mN·m-1, or about 45 mN·m-1 to about 50 mN·m-1 at 37° C. In some embodiments, the composition has a surface tension of about 15 mN·m-1, about 20 mN·m-1, about 30 mN·m-1, about 36 mN·m-1, about 41 mN·m-1, about 48 mN·m-1, about 54 mN·m-1, about 75 mN·m-1, about 40 mN·m-1, about 50 mN·m-1, about 60 mN·m-1, about 70 mN·m-1, about 80 mN·m-1, or a range of any two of these values at 37° C. In some embodiments, the composition has a surface tension of about 42 mN·m-1 to about 55 mN·m-1 at 37° C. In some embodiments, the composition has a surface tension of about 42 mN·m-1 to about 50 mN·m-1 at 37° C.

In some embodiments, a composition comprising greater than 40% w/w hydrogen peroxide has a surface tension of from about 35 mN·m-1 to about 60.3 mN·m-1 at 37° C. In some embodiments, a composition comprising greater than 40% w/w hydrogen peroxide may have a surface tension of about 60.3 mN·m-1 at 37° C. A composition comprising greater than 40% w/w hydrogen peroxide and 2.5% w/w 2-propanol may have a surface tension of about 54.1+/−0.8 mN·m-1 at 37° C. A composition comprising greater than 40% w/w hydrogen peroxide and 5% w/w 2-propanol may have a surface tension of about 48.3+/−0.7 mN·m-1 at 37° C. A composition comprising greater than 40% w/w hydrogen peroxide and 10% w/w 2-propanol may have a surface tension of about 41.1+/−0.6 mN·m-1 at 37° C. A composition comprising greater than 40% w/w hydrogen peroxide and 15% w/w 2-propanol may have a surface tension of about 35.9+/−0.6 mN·m-1 at 37° C.

In some embodiments, a composition comprising about 45% w/w hydrogen peroxide has a surface tension of from about 35 mN·m-1 to about 60.3 mN·m-1 at 37° C. In some embodiments, a composition comprising about 45% w/w hydrogen peroxide may have a surface tension of about 60.3 mN·m-1 at 37° C. A composition comprising about 45% w/w hydrogen peroxide and 2.5% w/w 2-propanol may have a surface tension of about 54.1+/−0.8 mN·m-1 at 37° C. A composition comprising about 45% w/w hydrogen peroxide and 5% w/w 2-propanol may have a surface tension of about 48.3+/−0.7 mN·m-1 at 37° C. A composition comprising about 45% w/w hydrogen peroxide and 10% w/w 2-propanol may have a surface tension of about 41.1+/−0.6 mN·m-1 at 37° C. A composition comprising about 45% w/w hydrogen peroxide and 15% w/w 2-propanol may have a surface tension of about 35.9+/−0.6 mN·m-1 at 37° C.

In some embodiments, the composition has a pH of about 1.5 to about 7.0. In some embodiments, the pH may be about 1.5 to about 3.5, about 1.5 to about 5.0, about 1.5 to about 4.0, about 1.7 to about 3.7, about 2.0 to about 5.0, about 2.0 to about 4.0, about 2.0 to about 2.8, about 2.5 to about 4.0, about 2.5 to about 4.5, about 2.5 to about 5.0, about 2.7 to about 3.83, about 2.7 to about 4.0, about 2.8 to about 4.0, about 2.83 to about 3.83, about 3.0 to about 7.0, about 4.0 to about 7.0, about 5.0 to about 7.0, or about 6.0 to about 7.0. In some embodiments, the pH may be about 1.5, 1.7, 2.0, 2.5, 2.7, 2.8, 2.83, 3.0, 3.3, 3.5, 3.7, 3.83, 4.0, 4.5, 5.0, 5.5, 6.0, 6.5, 7.0, or a range of any two of these values.

In some embodiments, the compositions of embodiments herein are stable for up to about four weeks at room temperature. In some embodiments, the compositions of embodiments herein are stable for up to about 15 minutes, about 30 minutes, about 1 hour, about 2 hours, about 3 hours, about 4 hours, about 5 hours, about 6 hours, up to about 12 hours, up to about 24 hours, up to about 1 week, up to about 2 weeks, up to about 3 weeks, up to about 1 month, up to about 6 weeks, up to about 2 months, up to about 3 months, up to about 4 months, up to about 6 months, up to about 8 months, up to about 10 months, up to about 12 months, up to about 18 months, up to about 2 years, up to about 2.5 years, or up to about 3 years at room temperature. In some embodiments, the compositions of embodiments herein are stable for up to about four weeks, up to about six weeks, up to about eight weeks, up to about three months, up to about 6 months, up to about 8 months, or up to about a year at 40° C. In some embodiments, the compositions of embodiments herein are stable for up to about four weeks, up to about 6 weeks, up to about 1 month, up to about 2 months, up to about 3 months, up to about 4 months, up to about 6 months, up to about 8 months, up to about 10 months, up to about 12 months, up to about 18 months, up to about 2 years, up to about 2.5 years, or up to about 3 years at 30° C. In some embodiments, the compositions of embodiments herein are stable for up to about four weeks, up to about 1 month, up to about 6 weeks, up to about 2 months, up to about 3 months, up to about 4 months, up to about 6 months, up to about 8 months, up to about 10 months, up to about 12 months, up to about 18 months, up to about 2 years, up to about 2.5 years, or up to about 3 years at 25° C. In some embodiments, the compositions of embodiments herein are stable for up to about four weeks, up to about 6 weeks, up to about 1 month, up to about 2 months, up to about 3 months, up to about 4 months, up to about 6 months, up to about 8 months, up to about 10 months, up to about 12 months, up to about 18 months, up to about 2 years, up to about 2.5 years, or up to about 3 years at 5° C. For example, for a composition comprising about 40° 0/w/w hydrogen peroxide and about 5% w/w 2-propanol was stable for 9 months at 5 and 25° C., and stable 6 months at 40° C. As an another example, for a composition comprising about 45% w/w hydrogen peroxide and 5% w/w 2-propanol was stable for 12 months at 5° C. and 25° C., and stable for 6 months at 40° C.

In some embodiments, the compositions of embodiments herein satisfy the stability requirements put forth by the International Conference of Harmonisation of Technical Requirements for Registration of Pharmaceuticals for Human Use in the “ICH Harmonised Tripartite Guideline: Stability Testing of New Drug Substances and Products Q1A(R2)”, current Step 4 version, dated 6 Feb. 2003, the entirety of which is incorporated herein by reference.

In some embodiments, the compositions of embodiments herein may also be administered in combination with other methods that mechanically, physically or chemically enhance the penetration of the active into the lesion or that otherwise may enhance the therapeutic efficacy of the composition. Such methods may include tape-stripping, destructive/ablative modalities such as, e.g. cryotherapy, electrosurgery, curettage, laser therapy, application of acids (e.g., salicylic acid, trichloroacetic acid); locally cytotoxic therapies, such as topical podophyllin, cantharidin, or topical or intralesional 5-fluorouracil, or bleomycin; topical immunomodulatory therapy (e.g., topical imiquimod, intralesional candida antigen, topical squaric acid dibutyl ester, oral cimetidine) or surgical lesion removal, electrodesiccation, lasers of various wavelengths (ablative and non-ablative), radio-frequency ablation, dermabrasion, and partial or complete surgical removal by curettage or surgical excision, use of an ablative or chemical peeling agent, or otherwise disturbing the surface of, or decreasing the thickness or size or overall volume of, or increasing the surface area of the lesion. In some embodiments, the compositions of embodiments herein may be administered in combination with other active ingredients, such as, for example, adjuvants, inhibitors, or other compatible drugs or compounds where such combination is seen to be desirable or advantageous in achieving the desired effects of the methods described herein. Any other known treatment may be used in combination with the formulation to treat the skin diseases disclosed herein. For example, the composition may be administered in combination with an active pharmaceutical agent that is used to treat the conditions described herein. In some embodiments, the compositions of embodiments herein may be administered before, concomitant with, or after the administration of another compound to treat the lesion. In some embodiments, the composition further comprises a topically active pharmaceutical agent used to treat the conditions described herein. As an example, a method of treating warts may comprise administering salicylic acid to the lesion of a person in need thereof in the morning and administering a composition of embodiments herein to the lesion of a person in need thereof in the evening. Another example includes a method of treating warts comprising administering cryotherapy or a locally destructive therapy to the lesion in a health care setting, followed by one or a series of regular (e.g. daily or weekly) applications of one of the compositions described herein. As another example, a method of treating warts, condyloma acuminate, or mollusca, may comprise administering a topical retinoid to the lesion of a person in need thereof for a number of days and administering a composition of embodiments herein to the lesion of a person in need thereof after a number of days of pretreatment with the topical agent. Other possible examples will be readily apparent to one knowledgeable in the art. The other active or procedure may be administered or performed either before, after or concurrently with the compositions of embodiments herein.

Embodiments herein are also directed to a method of treating warts or a wart associated condition in a subject in need thereof comprising administering a composition of embodiments herein to a subject in need thereof. In some embodiments, the composition may be administered once daily, twice daily, weekly, twice a week, three times a week, every other day, monthly, every two months, every three months or as directed by the packaging or the physician to achieve the desired clinical result. In some embodiments, the composition is administered once weekly. In some embodiments, the composition is administered twice weekly. In some embodiments, the composition is administered once or at regular intervals for one week, two weeks, three weeks, four weeks, five weeks, six weeks, seven weeks, eight weeks, nine weeks, ten weeks, eleven weeks, twelve weeks, one month, two months, three months, four months, five months, six months, or a range of any two of these values. In some embodiments, the composition is administered for at least about one week, at least about two weeks, at least about three weeks, at least about four weeks, at least about five weeks, at least about six weeks, at least about seven weeks, at least about eight weeks, at least about nine weeks, at least about ten weeks, at least about eleven weeks, at least about twelve weeks, at least about one month, at least about two months, at least about three months, at least about four months, at least about five months, at least about six months, a range of any two of these values, or until the lesion is resolved. In some embodiments, the composition is administered until the lesion is resolved. In some embodiments, the composition may be administered once weekly for eight weeks. In some embodiments, the composition may be administered twice weekly for eight weeks. In some embodiments, the composition may be administered once daily for one week and then administered once weekly or twice weekly until the lesion is resolved. Any other number of combinations are possible and will be readily apparent to one skilled in the art.

The condition may be of infectious etiology. In some embodiments, the condition may be selected from Human Papilloma Virus induced lesions e.g., warts, common warts, recurrent warts, filiform warts, genital warts, external genital warts, persistent warts, periungual warts, subungual warts, plantar warts, mosaic warts, recalcitrant warts, treatment naïve warts, palmoplantar warts, flat warts, epidermodysplasia verruciformis related warts, anogenital warts, condyloma accuminatum, cervical dysplasias or neoplasias, e.g., cervical intraepithelial neoplasia (CIN); Poxvirus (Poxviridae) induced lesions e.g., molluscum contagiosum (molluscipox) or orf (parapox), or a combination thereof. In some embodiments, the subject achieves improvement or partial clearance of the skin lesion after treatment with the composition. In some embodiments, the subject achieves complete clearance of the skin lesion during the course of treatment with the composition. In some embodiments, the subject achieves complete clearance of the skin lesion after the course of treatment with the composition. In some embodiments, the lesion continues to improve and/or resolve after the course of treatment. In some embodiments, the subject may achieve improvement in or complete clearance of the condition and/or lesions surrounding or distant to the site of treatment. Without wishing to be bound by theory, it is believed that treatment with the compositions herein may induce a local or systemic immunologic response to the virus responsible the condition.

In some embodiments, the method of treating warts or a wart associated condition further comprises the step of cleansing the treatment site before administration of the composition. In some embodiments, cleansing the treatment site comprises defatting the treatment site before the administration of the composition. In some embodiments cleansing the treatment site comprises applying at least 70% w/w 2-propanol to the skin to be treated before administration of the composition. Defatting the treatment site may comprise applying the alcohol onto the skin, such as by rubbing, massaging, placing, scrubbing, abrading, wiping, swabbing, or otherwise contacting the skin with the alcohol.

In some embodiments, the method of treating warts or a wart associated condition further comprises the step of debriding the treatment site of the subject. In some embodiments, debriding may include mechanically, chemically, or physically abrading, ablating, thinning, curetting, destroying, removing, excising, or otherwise disturb the surface of the skin or lesion to be treated, including decreasing the thickness of, and/or decreasing the volume of the lesion. In some embodiments, the step of debriding is before, after, and/or concurrent with administration of the compositions of embodiments herein to the treatment site.

In some embodiments, the alcohol may be selected from a primary alcohol, a secondary alcohol, a tertiary alcohol, or a combination thereof. The alcohol may be selected from methanol, ethanol, butanol, I-propanol, pentanol, hexanol, octanol, nonanol, decanol, 2-butanol, 2-propanol, 2-pentanol, 2-hexanol, benzyl alcohol, an isomer thereof, or a combination thereof. In some embodiments, defatting the skin may comprise applying another known defatting agent to the skin such as ethyl acetate, butyl acetate, or the like onto the skin, such as by rubbing, massaging, placing, scrubbing, abrading, wiping, swabbing, or otherwise contacting the skin with the agent. In some embodiments, cleansing the skin comprises applying an antiseptic solution to the skin or skin lesion to be treated. In some embodiments, the antiseptic is povidone, iodine, chlorhexidine, a detergent, soap or the like.

Some embodiments are directed to a method of treating warts comprising administering a composition of embodiments herein to a subject in need thereof. In some embodiments, the warts may be selected from warts, common warts, recurrent warts, persistent warts, filiform warts, genital warts, external genital warts, periungual warts, subungual warts, plantar warts, mosaic warts, recalcitrant warts, treatment naïve warts, palmoplantar warts, flat warts, epidermodysplasia verruciformis related warts, anogenital warts, condyloma accuminatum, cervical dysplasias or neoplasias. e.g., cervical intraepithelial neoplasia (CIN), or a combination thereof. In some embodiments, the composition comprises up to about 60% w/w hydrogen peroxide. In some embodiments, the composition comprises up to about 50% w/w hydrogen peroxide. In some embodiments, the composition comprises up to about 45% w/w hydrogen peroxide. In some embodiments, the composition comprises greater than 40% w/w to about 60% w/w hydrogen peroxide. In some embodiments, the composition comprises about 45% w/w hydrogen peroxide. In some embodiments, the composition comprises an alcohol and up to about 50% w/w hydrogen peroxide. In some embodiments, the composition comprises an alcohol and up to about 60% w/w hydrogen peroxide. In some embodiments, the composition comprises an alcohol and greater than 40% w/w to about 60% w/w hydrogen peroxide. In some embodiments, the alcohol is 2-propanol. In some embodiments, the alcohol is greater than 0% w/w to about 5% w/w 2-propanol. In some embodiments, the composition comprises about 25% w/w hydrogen peroxide and greater than 0% w/w to about 5% w/w 2-propanol. In some embodiments, the composition comprises about 32.5% w/w hydrogen peroxide and greater than 0% w/w to about 5% w/w 2-propanol. In some embodiments, the composition comprises greater than 40% w/w hydrogen peroxide and greater than 0% w/w to about 5% w/w 2-propanol. In some embodiments, the composition comprises about 41% w/w hydrogen peroxide and greater than 0% w/w to about 5% w/w 2-propanol. In some embodiments, the composition comprises about 42% w/w hydrogen peroxide and greater than 0% w/w to about 5% w/w 2-propanol. In some embodiments, the composition comprises about 43% w/w hydrogen peroxide and greater than 0% w/w to about 5% w/w 2-propanol. In some embodiments, the composition comprises about 44% w/w hydrogen peroxide and greater than 0% w/w to about 5% w/w 2-propanol. In some embodiments, the composition comprises about 45% w/w hydrogen peroxide and greater than 0% w/v to about 5% w/w 2-propanol. In some embodiments, the composition comprises about 46% w/w hydrogen peroxide and greater than 0% w/w to about 5% w/w 2-propanol. In some embodiments, the composition comprises about 47% w/w hydrogen peroxide and greater than 0% w/w to about 5% w/w 2-propanol. In some embodiments, the composition comprises about 48% w/w hydrogen peroxide and greater than 0% w/w to about 5% w/w 2-propanol. In some embodiments, the composition comprises about 49% w/w hydrogen peroxide and greater than 0% w/w to about 5° % w/w 2-propanol. In some embodiments, the composition comprises up to about 60% w/w hydrogen peroxide and 2-propanol.

In some embodiments, warts may be treated using any of the methods of treatment described herein. Some embodiments are directed to a method of improving the appearance of warts comprising administering a composition of embodiments herein to a subject in need thereof. Some embodiments are directed to a method of alleviating, e.g., shrinking, reducing in size, and/or reducing in height, a wart comprising administering a composition of embodiments herein to a subject in need thereof. In some embodiments, the warts are treatment naïve. In some embodiments, a treatment naïve wart may be treated using any of the methods of treatment described herein. Some embodiments are directed to a method of improving the appearance of treatment naïve warts comprising administering a composition of embodiments herein to a subject in need thereof. Some embodiments are directed to a method of improving the appearance of treatment naïve warts comprising administering a composition of embodiments herein to a subject in need thereof. Some embodiments are directed to a method of alleviating, e.g., shrinking, reducing in size, and/or reducing in height, a treatment naïve wart comprising administering a composition of embodiments herein to a subject in need thereof. In some embodiments, the warts are not treatment-naïve or are recalcitrant warts. Recalcitrant warts may include warts that are resistant to or have failed or have partially responded to other therapies, warts that are difficult to treat, or warts that are in anatomical locations that are difficult to treat or are known to have lower response to treatment or higher recurrence rates. In some embodiments, recalcitrant warts may include plantar warts, periungal warts, subungal warts or the like. In some embodiments, recalcitrant warts may be located on any body surface. In some embodiments, recalcitrant warts may be treated using any of the methods of treatment described herein. Some embodiments are directed to a method of improving the appearance of recalcitrant warts comprising administering a composition of embodiments herein to a subject in need thereof. Some embodiments are directed to a method of improving the appearance of recalcitrant warts comprising administering a composition of embodiments herein to a subject in need thereof. Some embodiments are directed to a method of alleviating, e.g., shrinking, reducing in size, and/or reducing in height, a recalcitrant wart comprising administering a composition of embodiments herein to a subject in need thereof. In some embodiments, the subject achieves improvement or partial clearance of the condition after treatment with the composition. In some embodiments, the subject achieves complete clearance of the warts after treatment with the composition. In some embodiments, the subject achieves a Physician Wart Assessment score of clear or mild after treatment with the composition. In some embodiments, the subject achieves a reduction of at least one ordinal on the Physician Wart Assessment score after treatment with the composition. In some embodiments, the subject achieves a reduction of at least two ordinals on the Physician Wart Assessment score after treatment with the composition. In some embodiments, the subject achieves a reduction of at least three ordinals on the Physician Wart Assessment score after treatment with the composition.

Some embodiments are directed to a method of treating Human Papilloma Virus induced lesions comprising administering a composition of embodiments herein to a subject in need thereof. Some embodiments are directed to a method of treating Poxvirus induced lesions comprising administering a composition of embodiments herein to a subject in need thereof. Some embodiments are directed to a method of treating molluscum contagiosum comprising administering a composition of embodiments herein to a subject in need thereof. In some embodiments, the subject achieves improvement or partial clearance of the condition after treatment with the composition. In some embodiments, the subject achieves complete clearance of the condition after treatment with the composition. In some embodiments, the method of treating molluscum contagiosum comprising administering a composition of embodiments herein to a subject in need thereof, wherein the hydrogen peroxide is in an amount of about 23% w/w to about 70% w/w.

Some embodiments herein are directed to a method of treating a skin condition comprising (i) topically administering a first dose of a composition of embodiments herein; and (ii) topically administering one or more follow-up doses of the composition. In some embodiments, the application of the first dose and one or more follow-up doses comprises one application session. In some embodiments, the follow-up dose is administered immediately after administration of the first dose. In some embodiments, the follow-up dose is administered at least about 0.5 minutes after the first dose. In some embodiments, the follow-up dose is administered at least about 1 minute, at least about 1.5 minutes, at least about 2 minutes, at least about 5 minutes, at least about 10 minutes, at least about 15 minutes, at least about 20 minutes, at least about 25 minutes, at least about 30 minutes, or at least about 1 hour after the first dose. In some embodiments, there may be one follow-up dose, two follow-up doses, three follow-up doses, four follow-up doses, five follow-up doses, six follow-up doses, seven follow-up doses, eight follow-up doses, nine follow-up doses or ten follow-up doses in each application session. Each subsequent follow-up dose may be administered immediately after administration of the previous dose, at least about 1 minute, at least about 1.5 minutes, at least about 2 minutes, at least about 5 minutes, at least about 10 minutes, at least about 15 minutes, at least about 20 minutes, at least about 25 minutes, at least about 30 minutes or at least one hour after the previous dose.

In some embodiments, the topical composition may be administered in an effective dose. In some embodiments, an effective dose may be from about 0.0025 milliliters to about 3 milliliters of the compositions of embodiments herein, including about 0.0025 milliliters, about 0.01 milliliters, about 0.025 milliliters, about 0.04 milliliters, about 0.05 milliliters, about 0.075 milliliters, about 0.1 milliliters, about 0.12 milliliters, about 0.15 milliliters, about 0.16 milliliters, about 0.5 milliliters, about 1 milliliters, about 1.5 milliliters, about 2 millimeters, about 2.5 milliliters, about 3 milliliters, 4 milliliters, 5 milliliters, 10 milliliters, 12 milliliters, a combination thereof, or any amount that has a clinical effect on the lesion. In some embodiments, the effective dose is proportional to size of the lesion. In some embodiments, the effective dose of the composition is less than about 0.12 milliliters per lesion. In some embodiments, an effective dose may be about 0.2 milliliters of the compositions of embodiments herein. In some embodiments, an effective dose may be from about 0.01 milliliters to about 3 milliliters of the compositions of embodiments herein. In some embodiments, an effective dose may be from about 0.01 milliliters to about 2 milliliters of the compositions of embodiments herein. In some embodiments, an effective dose may be from about 0.01 milliliters to about 1 milliliter of the compositions of embodiments herein. In some embodiments, an effective dose may be from about 0.01 milliliters to about 0.5 milliliters of the compositions of embodiments herein. In some embodiments, an effective dose may be from about 0.01 milliliters to about 0.4 milliliters of the compositions of embodiments herein. In some embodiments, an effective dose may be from about 0.01 milliliters to about 0.3 milliliters of the compositions of embodiments herein. In some embodiments, an effective dose may be from about 0.01 milliliters to about 0.2 milliliters of the compositions of embodiments herein. In some embodiments, an effective dose may be from about 0.02 milliliters to about 3 milliliters of the compositions of embodiments herein. In some embodiments, an effective dose may be from about 0.02 milliliters to about 2 milliliters of the compositions of embodiments herein. In some embodiments, an effective dose may be from about 0.02 milliliters to about 1 milliliter of the compositions of embodiments herein. In some embodiments, an effective dose may be from about 0.02 milliliters to about 0.5 milliliters of the compositions of embodiments herein. In some embodiments, an effective dose may be from about 0.02 milliliters to about 0.4 milliliters of the compositions of embodiments herein. In some embodiments, an effective dose may be from about 0.02 milliliters to about 0.3 milliliters of the compositions of embodiments herein. In some embodiments, an effective dose may be from about 0.02 milliliters to about 0.2 milliliters of the compositions of embodiments herein.

In some embodiments, topical administration of the first dose and the follow-up doses comprises massaging the composition into the skin for at least about 5 seconds, at least about 10 seconds, at least about 15 seconds, at least about 20 seconds, at least about 25 seconds, at least about 30 seconds, at least about 35 seconds, at least about 40 seconds, at least about 45 seconds, at least about 50 seconds, at least about 55 seconds, at least about 1 minute, at least about 2 minutes, at least about 3 minutes, at least about 4 minutes, or at least about 5 minutes, at least about 10 minutes, at least about 15 minutes, at least about 20 minutes at least about 25 minutes, or at least about 30 minutes during each dose. In some embodiments, the composition is a solution. In some embodiments, the composition is a gel. In some embodiments, massaging includes rubbing-in, manipulating, kneading, pressing, or otherwise “working” the composition into the skin. In some embodiments, the composition is applied and massaged into the skin with an applicator. In some embodiments, the composition is applied and massaged into the skin at least 1 time, at least 2 times, at least 3 times, at least 4 times, at least 5 times, at least 6 times, at least 7 times, at least 8 times, at least 9 times, or at least 10 times in each application session. In some embodiments, the application session, in which the composition is administered, may be repeated once daily, twice daily, weekly, biweekly, three times a week, every other day, monthly, every two months, every three months, every six months or as directed by the packaging or the physician to achieve the desired clinical result.

In some embodiments, the composition is administered by a health care provider. In some embodiments, the composition is administered in a health care setting. In some embodiments, the composition is self-administered by the subject in need thereof. In some embodiments, the composition is administered by a caregiver of the subject. As used herein, health care provider may include doctor, nurse, physician's assistant, nurse practitioner, medical assistant, or any professional working in a doctor's office, hospital or clinic. A health care setting, as used herein, refers to a doctor's office, hospital, ambulatory care setting, or clinic. A caregiver of the subject may include parents, nurse, friend, family member, medical professional, or anybody assisting in administering the composition to the subject in need thereof.

In some embodiments, the method further comprises topically administering a second composition comprising hydrogen peroxide and alcohol as described in embodiments above to the subject following an initial treatment by a health care provider. In some embodiments, the second composition is a take home composition. In some embodiments, the second composition is available over the counter. In some embodiments, the second composition is available by prescription. In some embodiments, the second composition may comprise a lower concentration of hydrogen peroxide than the initial treatment administered to the subject. In some embodiments, the second composition may comprise the same concentration of hydrogen peroxide as the initial treatment administered to the subject. In some embodiments, the second composition comprises up to about 60% w/w hydrogen peroxide and an alcohol. In some embodiments, the second composition comprises about 45% w/w hydrogen peroxide and an alcohol. In some embodiments, the second composition comprises greater than 40% w/w hydrogen peroxide and 5% w/w alcohol. In some embodiments, the second composition comprises greater than 40% w/w to about 60% w/w hydrogen peroxide and 5% w/w alcohol. In some embodiments, the second composition comprises about 45% w/w hydrogen peroxide and 5% w/w alcohol. In some embodiments, the second composition comprises about 50% w/w hydrogen peroxide and 5% w/w alcohol. In some embodiments, the second composition comprises about 54% w/w hydrogen peroxide and 5% w/w alcohol. In some embodiments, the second composition comprises about 55% w/w hydrogen peroxide and 5% w/w alcohol. In some embodiments, the second composition comprises about 59% w/w hydrogen peroxide and 5% w/w alcohol. In some embodiments, the second composition comprises about 40% w/w hydrogen peroxide and 5% w/w alcohol. In some embodiments, the second composition comprises about 35% w/w hydrogen peroxide and 5% w/w alcohol. In some embodiments, the second composition comprises about 32.5% w/w hydrogen peroxide and 5% w/w alcohol. In some embodiments, the second composition comprises about 25% w/w hydrogen peroxide and 5% w/w alcohol. In some embodiments, the hydrogen peroxide in the second composition may be in an amount of about 0.5% w/w, about 5% w/w, about 10% w/w, about 15% w/w, about 20% w/w, about 23.5% w/w, about 25% w/w, about 30% w/w, about 32.5% w/w, about 35% w/w, about 40% w/w, about 41% w/w, about 42% w/w, about 43% w/w, about 44% w/w, about 45% w/w, about 46% w/w, about 47% w/w, about 48% w/w, about 49% w/w, about 50% w/w, about 51% w/w, about 52% w/w, about 53% w/w, about 54% w/w, about 55% w/w, about 56% w/w, about 57% w/w, about 58% w/w, about 59% w/w, about 60% w/w, about 65% w/w, about 70% w/w, or a range of any two of these values. In some embodiments, the alcohol is 2-propanol. In some embodiments, the second composition may be self-administered by the subject. In some embodiments, the second composition may be administered at least about 1 day, at least about 2 days, at least about 3 days, at least about 1 week, at least about 2 weeks, at least about 3 weeks, at least about 4 weeks, at least about 5 weeks, at least about 6 weeks, at least about 7 weeks or at an interval required to maintain the clinical effect or until the lesion is cleared, after the initial treatment by the healthcare professional. In some embodiments, the second composition may be administered once daily, twice daily, weekly, biweekly, every other day, monthly, every two months, every three months, every six months, or as directed by the packaging or the physician to achieve the clinically desired result.

Some embodiments herein are directed to a method of treating a skin condition in a subject comprising topically administering a take home composition comprising hydrogen peroxide and alcohol as described above to the subject. In some embodiments, the take home composition is available over the counter. In some embodiments, the take home composition is available by prescription. In some embodiments, the take home composition comprises up to about 60% w/w hydrogen peroxide and an alcohol. In some embodiments, the take home composition comprises about 60% w/w hydrogen peroxide and an alcohol. In some embodiments, the take home composition comprises about 50% w/w hydrogen peroxide and an alcohol. In some embodiments, the take home composition comprises about 55% w/w hydrogen peroxide and an alcohol. In some embodiments, the take home composition comprises about 45% w/w hydrogen peroxide and 5% w/w alcohol. In some embodiments, the take home composition comprises about 40% w/w hydrogen peroxide and 5% why alcohol. In some embodiments, the take home composition comprises about 35% w/w hydrogen peroxide and 5% w/w alcohol. In some embodiments, the take home composition comprises about 32.5% w/w hydrogen peroxide and 5% w/w alcohol. In some embodiments, the take home composition comprises about 25% w/w hydrogen peroxide and 5% w/w alcohol. In some embodiments, the alcohol is 2-propanol. Such take home compositions may be administered following a visit to a healthcare professional. In some embodiments, the take home composition may be administered following an initial treatment by a healthcare professional. In some embodiments, the take home composition may comprise a lower concentration of hydrogen peroxide than the initial treatment administered to the subject. In some embodiments, the take home composition may comprise the same concentration of hydrogen peroxide as the initial treatment administered to the subject. The take home composition may be self-administered by the subject. In some embodiments, the take home composition may be administered at least about 1 day, at least about 2 days, at least about 3 days, at least about 1 week, at least about 2 weeks, at least about 3 weeks, at least about 4 weeks after the initial treatment by the healthcare professional. In some embodiments, the take home composition may be administered once daily, twice daily, weekly, biweekly, every other day, monthly, every two months, every three months, every six months, or as directed by the packaging or the physician to achieve the clinically desired result. In some embodiments, the take home composition may be administered for a period of one day, one week, two weeks, three weeks, four weeks, five weeks, six weeks, seven weeks, eight weeks, nine weeks, ten weeks, eleven weeks, twelve weeks, or for any duration of time as directed by the packaging or the physician to achieve the clinically desired result.

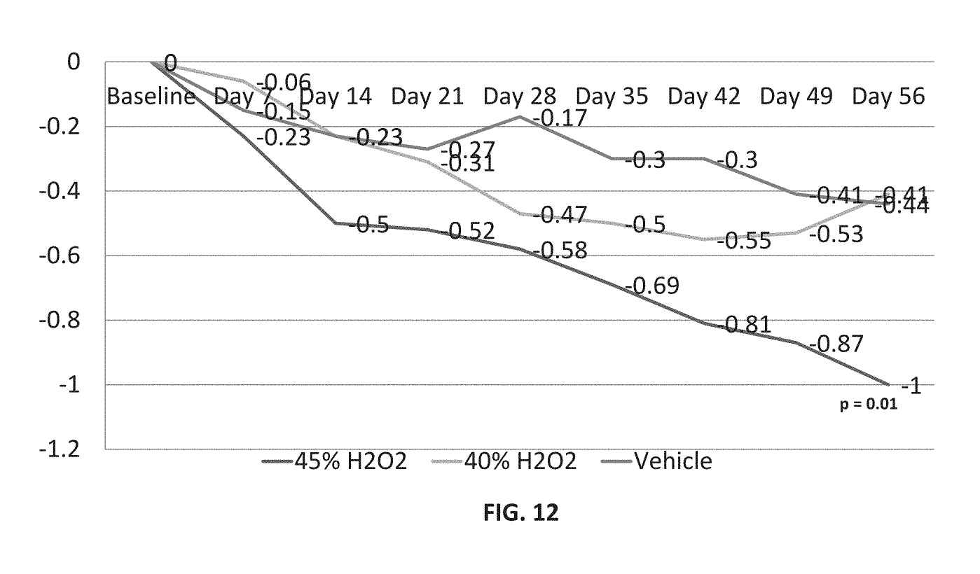

Embodiments herein also encompass devices for administering the hydrogen peroxide compositions of embodiments herein (see, for example, FIGS. 12-16 and associated description). In some embodiments, a composition comprising hydrogen peroxide and alcohol as described in embodiments herein may be administered using any topical applicator known in the art. In some embodiments, a composition comprising hydrogen peroxide and alcohol as described in embodiments herein may be administered using a sponge, a swab, a foam tipped stick, a cotton ball, a brush, a woven or non-woven fabric, roller, gauze, a pen, a glove, or the like. In some embodiments, the applicator may dispense compositions of embodiments herein via a tip that is flocked, absorbent, and/or firm enough to apply pressure to the lesion. In some embodiments the applicator is a sintered polymeric-tip applicator. In some embodiments, the applicator is resistant to, compatible with, or inert to high concentration peroxide solutions. In some embodiments, the applicator is capable of dispensing the solution at a controlled rate in order to help confine the active to the lesion of interest and spare surrounding normal skin. In some embodiments, the solution may be administered using a device having the hydrogen peroxide solution in a compartment that dispenses the solution onto or through an applicator tip when needed. In some embodiments, the applicator may comprise a material designed to abrade the skin lesion or treatment site before, after, or at the time of administration of the composition. In some embodiments, the applicator is a doe footed applicator. In some embodiments, the applicator is a flocked, doe footed applicator. In some embodiments, the flocked, doe footed applicator is comprised of HDPE (high density polyethylene), nylon, and an adhesive. In some embodiments, the solution is applied once daily, twice daily, weekly, biweekly, three times a week, every other day, monthly, every two months, every three months or as directed by the packaging or the physician or provider to achieve the desired clinical result.

In some embodiments, a method of treating a skin condition comprises administering the composition of embodiments herein. In some embodiments, administering the composition of embodiments herein comprises contacting a treatment site with a tip of an applicator, wherein the applicator comprises a suitable container having the composition disposed therein. In some embodiments, administering the composition of embodiments herein comprises contacting a treatment site with a tip of an applicator, wherein the applicator comprises a frangible ampoule having the composition disposed therein, an applicator body having the frangible ampoule arranged therein, an applicator hub in fluid communication with the applicator body, the tip arranged at a proximal end of the applicator hub, and a filter arranged between the frangible ampoule and the tip. In some embodiments, administering the topical composition comprises controlling a flow rate of the topical composition out of the tip by varying a squeezing force applied to the exterior surface of the applicator body. In some embodiments, administering the topical composition comprises contacting the tip with a targeted lesion of the skin condition whereby the topical composition dispenses through the tip onto the targeted lesion. In some embodiments, administering the topical composition further comprises applying pressure to a pressure area arranged on an outer surface of the applicator body causing the frangible ampoule to rupture. In some embodiments, administering the topical composition comprises causing the frangible ampoule to rupture and release the topical composition through the tip; and contacting the tip to a targeted lesion of the skin condition.

Some embodiments herein are directed to a kit for the treatment of a skin condition comprising a container comprising a compartment having hydrogen peroxide, a compartment having 2-propanol and instructions for use. Some embodiments herein are directed to a kit for the treatment of a skin condition comprising a container comprising hydrogen peroxide and 2-propanol and instructions for use. Some embodiments are directed to a kit for the treatment of a skin condition comprising a container comprising hydrogen peroxide and 2-propanol and instructions for use. In some embodiments, the kit further comprises an applicator. In some embodiments, the kit may include two or more applicators. For example, in some embodiments, a kit may contain many applicators where there is more than one lesion to treat (for example, in an over the counter kit or a kit for multiple administrations by the physician) or if the kit further includes a take home formulation of the composition for multiple applications. In some embodiments, the kit may comprise an applicator with a frangible glass ampoule having a solution of hydrogen peroxide and an alcohol. In some embodiments, the kit may comprise an applicator with a frangible glass ampoule having a solution of hydrogen peroxide, wherein the applicator also comprises a compartment having an alcohol, such as, without limitation, 2-propanol, in the applicator body. In some embodiments, the applicator may further include a compartment having a gelling agent. In some embodiments, the kit may comprise two or more containers of the topical hydrogen peroxide formulation. In some embodiments, the two or more containers of the topical hydrogen peroxide formulation may comprise hydrogen peroxide formulations of the same hydrogen peroxide concentration. In some embodiments, the two or more containers of the topical hydrogen peroxide formulation may comprise hydrogen peroxide formulations of different hydrogen peroxide concentrations. For example, in some embodiments, the kit may comprise a container comprising greater than 40% w/w hydrogen peroxide and 5% w/w 2-propanol for administration by the physician in the office, and other containers of different concentrations (e.g., weaker concentrations) of hydrogen peroxide for the patient to take home for subsequent applications to the target site. In some embodiments, a kit may include two or more single-use containers of the topical hydrogen peroxide formulation with multiple applicators. In some embodiments, a kit may include a container having multiple dose containers of the topical hydrogen peroxide formulation with multiple applicators. The skin condition may be a virally induced or non-virally induced cutaneous growth or proliferation. In some embodiments, the skin condition may be selected from Human Papilloma Virus induced lesions e.g., warts, common warts, palmoplantar warts, flat warts, recurrent warts, filiform warts, external genital warts, genital warts, recalcitrant warts, treatment naïve warts, epidermodysplasia verruciformis related warts, anogenital warts, condyloma accuminatum, cervical dysplasias or neoplasias, e.g., cervical intraepithelial neoplasia (CIN); Poxvirus induced lesions e.g., molluscum contagiosum or orf, or a combination thereof. In some embodiments, the applicator may be selected from a sponge, a swab, a foam tipped stick, a cotton ball, a brush, a woven or non-woven fabric, roller, gauze, a pen, or the like. In some embodiments, the applicator is flocked, absorbent, and/or firm enough to apply pressure to the lesion. In some embodiments the applicator is a sintered polymeric-tip applicator. In some embodiments, the applicator is resistant to, compatible with, or inert to high concentration peroxide solutions. In some embodiments, the applicator is capable of dispensing the solution at a controlled rate in order to help confine the active to the lesion of interest and spare surrounding normal skin. In some embodiments, the solution may be administered using a device having the hydrogen peroxide solution in a compartment that dispenses the solution onto or through an applicator tip when needed. In some embodiments, the applicator may comprise a material or feature designed to abrade or debride the skin lesion or treatment site before or at the time of administration of the composition. In some embodiments, the applicator has a material or feature to mechanically or physically enhance penetration of the hydrogen peroxide into a lesion or otherwise enhance therapeutic efficacy of the composition. In some embodiments, the material or feature of the applicator is designed to sand, file, curette, abrade, or debride a treatment site of the subject before administration of the topical composition.

In some embodiments, the applicator is a doe footed applicator. In some embodiments, the applicator is a flocked, doe footed applicator. In some embodiments, the flocked, doe footed applicator is comprised of HDPE (high density polyethylene), LDPE (low density polyethylene), Nylon, an adhesive, or any combination thereof. In some embodiments, the container may be selected from a vial, an ampule, ajar, a bottle, a medication tube, a syringe, or any other container for storing liquids. In some embodiments, the compartment may be selected from a vial, a tube, an ampoule, a jar, a bottle, a medication tube, a syringe, or any other container for storing liquids. In some embodiments the container may be glass, borosilicate glass, Type I borosilicate glass, tinted or otherwise light protected glass, amber tinted glass, amber Type I borosilicate glass, HDPE, Teflon®, silicone, ABS (acrylonitrilebutadiene stylene), or other compatible polymer or material. In some embodiments, the kit may include a glass vial or bottle having a solution of hydrogen peroxide and an alcohol disposed therein. In some embodiments the glass vial or bottle may include an amber glass vial or bottle. In some embodiments, the kit may comprise a vial or bottle at least partially formed from HDPE having a solution of hydrogen peroxide and an alcohol disposed therein. In some embodiments, the container may be formed from a material that has a low reactivity with peroxide. In some embodiments, the compartment may include a material that has a low reactivity with peroxide. In some embodiments, the hydrogen peroxide is a stabilized hydrogen peroxide. In some embodiments, the hydrogen peroxide in the one or more containers of the kit may be in an amount of any embodiment described herein, including, for example, greater than 40% w/w, about 45% w/w, about 50% w/w, about 54% w/w, about 55% w/w, about 59% w/w, about 60% w/w. In some embodiments, the alcohol, such as, without limitation, 2-propanol, is in an amount of less than about 25% w/w, including, for example, about 5% w/w or about 2.5% w/w. In some embodiments, the kit may be for use by a health care provider. In some embodiments, the kit may be for use by the subject in need thereof. In some embodiments, the kit may be available only with a prescription. In some embodiments, the kit may be available over the counter for use by the subject in need thereof.

Some embodiments herein are directed toward devices for administering the compositions of embodiments herein. In some embodiments, the devices may include an applicator configured to safely and effectively deliver the compositions of embodiments herein to the targeted skin of a patient. Some embodiments herein include an applicator configured to store and dispense a topical composition comprising an agent selected from a caustic agent, an unstable agent or a combination thereof, the applicator comprising a frangible ampoule having the agent disposed therein, an applicator body having the frangible ampoule arranged therein; an applicator hub in fluid communication with the applicator body; a tip arranged at a proximal end of the applicator hub; and a filter arranged between the frangible ampoule and the tip. In some embodiments, the topical composition comprises from about 40% w/w to about 60% w/w hydrogen peroxide and greater than 0% w/w to about 5% w/w 2-propanol. In some embodiments, the agent comprises from about 40% w/w hydrogen peroxide to about 60% w/w hydrogen peroxide. In some embodiments, the frangible ampoule further includes greater than 0% w/w to about 5% w/w 2-propanol. In some embodiments, the applicator body further comprises an additional ingredient of the topical composition disposed therein, whereby the agent is released from the frangible ampoule responsive to the frangible ampoule being ruptured and is mixed with the additional ingredient in the applicator body prior to administration of the topical composition. In some embodiments, the topical composition is released from the frangible ampoule responsive to the frangible ampoule being ruptured and flows through the applicator body, the filter, and out of the applicator through the tip. In some embodiments, the applicator further includes a pressure area arranged on an outer surface of the applicator body to indicate a portion of the applicator body to apply pressure to rupture the frangible ampoule.

Some embodiments are directed to an applicator configured to store and dispense a topical composition comprising from about 25% w/w to about 60% w/w hydrogen peroxide and greater than 0% w/w to about 5% w/w 2-propanol, the applicator comprising a frangible ampoule having the topical composition disposed therein, an applicator body having the frangible ampoule arranged therein, an applicator hub in fluid communication with the applicator body, a tip arranged at a proximal end of the applicator body; and a filter arranged between the frangible ampoule and the tip. In some embodiments, the frangible ampoule is formed from at least one of glass, plastic, borosilicate glass, Type 1 borosilicate glass, and tinted glass. In some embodiments, the applicator body is formed from polypropylene, high-density polyethylene, low-density polyethylene, polyvinyl chloride, polyethylene, or a combination thereof. In some embodiments, the filter is configured to prevent shards of a ruptured frangible ampoule from passing through and to allow the topical composition to flow through. In some embodiments, the filter is formed from at least one of polypropylene, high-density polyethylene, low-density polyethylene, polyethylene, polystyrene, a ceramic material, a foam material, sand, diatomaceous earth, and paper fibers.

Some embodiments are directed to a method of treating a skin condition, the method comprising administering a topical composition comprising up to about 60% w/w hydrogen peroxide and greater than 0% w/w to about 5% w/w 2-propanol using an applicator comprising a frangible ampoule having the topical composition disposed therein, an applicator body having the frangible ampoule arranged therein, an applicator hub in fluid communication with the applicator body, a tip arranged at a proximal end of the applicator hub, and a filter arranged between the frangible ampoule and the tip. In some embodiments, administering the topical composition comprises applying a squeezing force to the exterior surface of the applicator body to dispense the composition. In some embodiments, administering the topical composition comprises contacting the tip with a targeted lesion of the skin condition whereby the topical composition dispenses through the tip onto the targeted lesion. In some embodiments, administering the topical composition further comprises applying pressure to a pressure area arranged on an outer surface of the applicator body causing the frangible ampoule to rupture. In some embodiments, administering the topical composition comprises causing the frangible ampoule to rupture and release the topical composition through the tip; and contacting the tip to a targeted lesion of the skin condition.

In some embodiments, the applicator may include a frangible ampoule configured to store the compositions of embodiments herein arranged within an applicator body. In some embodiments, the ampoule may be formed from glass or other similar frangible materials, such as borosilicate glass. In some embodiments, the applicator body may be formed from various flexible materials, including, without limitation high-density polyethylene (HDPE), low-density polyethylene (LDPE) or various combinations or blends thereof. In some embodiments, the compositions of embodiments herein may be released from the ampoule by applying manual pressure (for instance, “squeezing”) on the applicator body in a direction toward the ampoule sufficient to cause the ampoule to break apart. In some embodiments, the compositions of embodiments herein released from the ampoule may flow through a filter configured to filter glass shards from the broken ampoule while allowing the compositions of embodiments herein to flow therethrough. In some embodiments, at least one portion of the applicator may include a hydrophobic material, such as the filter and/or the tip. In some embodiments the hydrophobic material is composed of polyester or co-polyester polymers, acrylic, modified acrylic (e.g., modacrylic), polypropylene, polyethylene or combinations or mixtures thereof. Non-limiting examples of hydrophobic materials may also include materials including, coated with, and/or modified by silanes, alkylsilanes, fluoroalkylsilanes, silicone, combinations thereof, and derivatives thereof. In some embodiments, the hydrophobic portions of the applicator may operate to impede, reduce, restrict, prevent, and/or substantially prevent the compositions of embodiments herein that have been released from the ampoule from flowing through portions of the applicator and/or out of the tip of the applicator. In some embodiments, the compositions of embodiments herein may flow through the filter and out of the applicator via an applicator tip for application on the skin of a patient. In some embodiments, the tip may include a flocked portion formed from filaments of various chemically compatible and non-reactive materials, such as nylon.

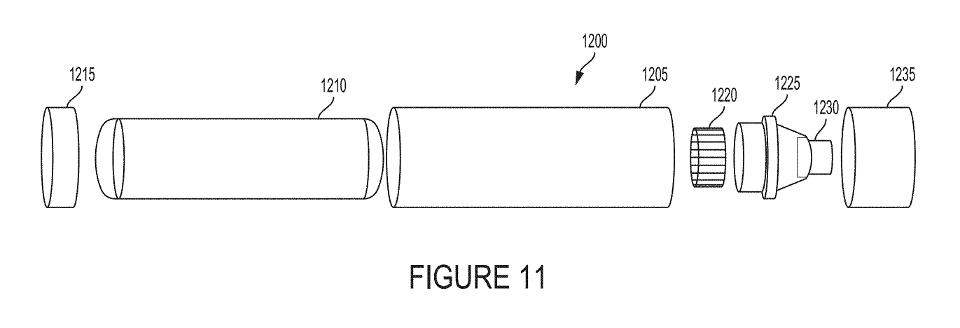

FIG. 11 depicts an illustrative applicator configured according to a first embodiment. As shown in FIG. 11, the applicator 1200 may include an applicator body 1205. In some embodiments, the applicator tube 1205 may be enclosed at a distal end thereof by an end cap 1215. In some embodiments, the applicator body 1205 and/or the end cap 1215 may be formed from various flexible materials, including, without limitation, flexible polymer materials. Non-limiting examples of flexible polymer materials may include polypropylene (PP), high-density polyethylene (HDPE) or low-density polyethylene (LDPE), polyvinyl chloride (PVC), polyethylene (PE), derivatives thereof, and any combination thereof. In some embodiments, the applicator body 1205 may have a generally longitudinal shape. In some embodiments, the applicator body 1205 may have a generally cylindrical shape. The applicator body 1205 may have various lengths according to some embodiments, including about 80 millimeters, about 90 millimeters, about 98 millimeters, about 99 millimeters, about 100 millimeters, about 110 millimeters, about 120 millimeters, about 130 millimeters, about 140 millimeters, about 150 millimeters, about 80 millimeters to about 100 millimeters, about 80 millimeters to about 150 millimeters, about 90 millimeters to about 100 millimeters, about 100 millimeters to about 120 millimeters, about 100 millimeters to about 150 millimeters, and any values or ranges between any of these values (including endpoints). The applicator body 1205 may have various outer dimensions according to some embodiments, including about 5 millimeters, about 7 millimeters, about 9 millimeters, about 10 millimeters, about 12 millimeters, about 15 millimeters, about 18 millimeters, about 20 millimeters, about 5 millimeters to about 7 millimeters, about 5 millimeters to about 10 millimeters, about 7 millimeters to about 10 millimeters, about 9 millimeters to about 15 millimeters, about 10 millimeters to about 15 millimeters, about 5 millimeters to about 20 millimeters, about 10 millimeters to about 20 millimeters, and any values or ranges between any of these values (including endpoints).

In some embodiments, an ampoule 1210 configured to store compositions of embodiments herein may be arranged within a cavity formed within the applicator body 1205. In some embodiments, the ampoule 1210 may be frangible, for example, configured to shatter, fragment, rupture, fracture, or otherwise break apart responsive to the application of sufficient pressure thereto. For instance, sufficient pressure may be applied manually, such as by applying a force by a human hand (for example, by squeezing or pressing) on the applicator body 1205 in a direction toward the ampoule 1210 in an area of the applicator body where the ampoule is located. In this manner, the force applied to the applicator body 1205 may be transferred to the ampoule 1210 and the ampoule may break apart responsive to the application of sufficient force thereto. In some embodiments, the ampoule 1210 may be formed from various frangible materials, including glass, plastic, a polymer material, borosilicate glass, Type I borosilicate glass, tinted glass, and any combination thereof. In some embodiments, the ampoule 1210 may include one or more weakened regions to facilitate breakage of the ampoule 1210. In some embodiments, the compositions of embodiments herein may be caustic and/or may react with the materials of the applicator 1200 or the environment. In some embodiments, the compositions of embodiments herein may be degraded or otherwise effected by contaminants and/or contact with the environment. Accordingly, storage of compositions of embodiments herein using one or more ampoules 1210 may protect the applicator 1200 materials and/or the compositions of embodiments herein from being degraded until they are released from the ampoule. Through the use of the ampoules 1210, the applicator 1200 may, among other things, allow for the safe and stable storage of compositions of embodiments herein and other components until delivery is required.

The compositions of embodiments herein stored within the ampoule 1210 may be released within the applicator body 1205 responsive to the fracturing of the ampoule 1210. The ampoule 1210 may be configured to hold various volumes of the compositions of embodiments herein, including, about 0.1 milliliters, about 0.2 milliliters, about 0.5 milliliters, about 1 milliliters, about 1.5 milliliters, about 2 milliliters, about 2.4 milliliters, about 3 milliliters, about 3.2 milliliters, about 4 milliliters, about 5 milliliters, about 10 milliliters, about 20 milliliters, about 30 milliliters, about 50 milliliters, about 100 milliliters, about 500 milliliters, about 0.1 milliliters to about 3 milliliters, about 0.1 milliliters to about 4 milliliters, about 0.1 milliliters to about 5 milliliters, about 0.1 milliliters to about 10 milliliters, about 0.1 milliliters to about 100 milliliters, about 0.1 milliliters to about 500 milliliters, about 1 milliliters to about 3 milliliters, about 1 milliliters to about 4 milliliters, about 1 milliliters to about 5 milliliters, about 1 milliliters to about 100 milliliters, about 1 milliliters to about 500 milliliters, about 2 milliliters to about 3 milliliters, about 2 milliliters to about 4 milliliters, about 2 milliliters to about 5 milliliters, about 2 milliliters to about 10 milliliters, about 3 milliliters to about 4 milliliters, about 3 milliliters to about 5 milliliters, about 3 milliliters to about 10 milliliters, about 3 milliliters to about 100 milliliters, about 4 milliliters to about 5 milliliters, about 4 milliliters to about 10 milliliters, about 4 milliliters to about 100 milliliters, about 5 milliliters to about 10 milliliters, about 5 milliliters to about 100 milliliters, about 100 milliliters to about 500 milliliters, and any values or ranges between any of these values (including endpoints). In some embodiments, the ampoule 1210 may be configured to hold an effective dose of the compositions of embodiments herein. In some embodiments, the ampoule 1210 may be configured to hold an effective dose of the compositions of embodiments herein sufficient to treat a single target lesion or skin area. In some embodiments, the ampoule 1210 may be configured to hold an effective dose of the compositions of embodiments herein sufficient to treat multiple target lesions or skin areas. In some embodiments, an effective dose may be from about 0.0025 milliliters to about 3 milliliters of the compositions of embodiments herein, including about 0.0025 milliliters, about 0.01 milliliters, about 0.025 milliliters, about 0.05 milliliters, about 0.075 milliliters, about 0.1 milliliters, about 0.15 milliliters, about 0.5 milliliters, about 1 milliliters, about 1.5 milliliters, about 2 millimeters, about 2.5 milliliters, about 3 milliliters, a combination thereof, or any amount that has a clinical effect on the lesion. In some embodiments, the effective dose is proportional to size of the lesion. In some embodiments, the effective dose of the composition is less than about 0.1 milliliters per lesion. In some embodiments, an effective dose may be about 3 milliliters of the compositions of embodiments herein. In some embodiments, an effective dose may be from about 2 milliliters to about 3 milliliters of the compositions of embodiments herein. In some embodiments, an effective dose may be from about 2 milliliters to about 5 milliliters of the compositions of embodiments herein.

Although only one ampoule 1210 is depicted in FIG. 11, embodiments are not so limited, as the applicator 1200 may include a plurality of ampoules. For instance, an applicator 1200 may include multiple ampoules 1210 for multiple doses of the compositions of embodiments herein. In another instance, an applicator 1200 may include a plurality of ampoules 1210 housing different compositions. In some embodiments, the applicator 1200 may include a compartment (not shown) housing a first composition (e.g., a gelling agent, hydrogen peroxide) and one or more ampoules 1210 housing one or more second compositions (e.g., 2-propanol, compositions of embodiments herein). For example, one or more walls may partition the applicator body into one or more compartment portions. In some embodiments, a compartment or a portion thereof (e.g., a wall portion forming the compartment within the applicator body 1205) may be fractured by the application of sufficient force to the compartment. In some embodiments, one or more first compositions may be disposed within the applicator body 1205 such that one or more second compositions disposed within the one or more ampoules 1210 and/or one or more compartments may contact the one or more second compositions responsive to the release of the one or more second compositions. A non-limiting example of a first composition may include an alcohol (e.g., 2-propanol) or a gelling agent and a non-limiting example of a second composition may include a caustic formulation, such as a hydrogen peroxide solution. In some embodiments, the various compositions (e.g., the one or more first compositions and the one or more second compositions) may be mixed together before or concurrently with the application thereof.

In some embodiments, an applicator hub 1225 may be in fluid communication with the applicator body 1205 at a proximal end of the applicator 1200. The applicator hub 1225 may be configured to receive the compositions of embodiments herein that flow from the applicator body 1205 responsive to the release of the compositions of embodiments herein from the ampoule 1210. In some embodiments, at least a portion of the applicator hub 1225 may be arranged within the applicator body 1205. In some embodiments, the applicator hub 1225 may be fixedly attached to the applicator body 1205, such as through the use of adhesives, a friction fit, a press-fit, a threaded fit, or the like. In some embodiments, an outer portion of the applicator hub 1225 may be fixedly attached to an inner portion of the proximal end of the applicator body 1205. In some embodiments, the applicator hub 1225 may form a hermetic seal with the applicator body 1205 through a friction fit, adhesives, a threaded fit, or the like. In some embodiments, the applicator hub 1225 may be formed from a polymer material, including, without limitation. PE, PP, HDPE, and LDPE. In some embodiments, a protective cap 1235 may be configured to enclose the applicator hub 1225 at a proximal end of the applicator 1200.

In some embodiments, a filter 1220 may be arranged within the applicator 1200 between the ampoule 1210 and the proximal end of the applicator 1200. In some embodiments, the filter 1220 may be arranged within a distal portion of the applicator hub 1225 that faces the ampoule 1210. The filter 1220 may be configured to filter out pieces of the broken ampoule 1210 while allowing the compositions of embodiments herein to flow therethrough. The filter 1220 may be formed from various materials, including any material capable of filtering out the shards of the ampoule from the compositions of embodiments herein. Non-limiting examples of filter 1220 materials may include various polymer materials, PE, PP, HDPE, LDPE, polystyrene (PS), carbon, a foam material, ceramic, sand, diatomaceous earth (DE), paper fibers, and any combination thereof. In some embodiments, the filter 1220 material may be selected to provide a particular flow rate of the compositions of embodiments herein through the filter.

In some embodiments, the applicator 1200 may include a tip 1230 arranged at a proximal portion thereof. In some embodiments, the tip 1230 may be fixedly arranged within a central bore of the applicator hub 1225. The tip 1230 may be configured to facilitate the application of the compositions of embodiments herein onto the skin of a patient. In some embodiments, the tip 1230 may be formed from an absorbent or substantially absorbent material. In some embodiments, the tip 1230 may be configured as a sintered polymeric tip. In some embodiments, the tip 1230 may be configured as a doe footed tip. In some embodiments, the tip 1230 may be configured as a flocked tip, for example, that may include and/or may be formed from a flocked material, including, without limitation flock formed from filaments of various materials. In some embodiments, the flocked tip may be formed by affixing a flocked material to the tip 1230. In some embodiments, the tip 1230 may be formed from various materials that are non-reactive or otherwise chemically compatible with the compositions of embodiments herein. In some embodiments, the tip 1230 may be formed from various biocompatible materials. In some embodiments, the tip 1230 may be formed from various materials, including, without limitation, cellulose, nylon, rayon, polyester, Teflon®, fibers thereof, flocked materials thereof, and any combination thereof.

In some embodiments, the tip 1230 may be fixedly arranged within the applicator 1200, such as in the applicator hub 1225, such as through adhesives, a friction fit, a threaded fit, a snap or lock fit, a press-fit, or the like. In some embodiments, the applicator 1200 may be configured to allow replacement of the tip 1230 during or after each use. In some embodiments, the ampoule 1210 may be replaced within the applicator 1200. For instance, an operator may remove the end cap 1215, remove the shards of a fractured ampoule 1210, and insert a new ampoule with the same and/or different compositions of embodiments herein. In this manner, the applicator 1200 may be used multiple times and/or for the treatment of multiple skin conditions (i.e., separate lesions, warts, or other skin formations) of the same patient. In some embodiments, the applicator 1200 may be a single use applicator. In some embodiments, the single use applicator is intended to be discarded after a single use.

In some embodiments, the compositions of embodiments herein may be released from the ampoule 1210 and flow through the applicator body 1205 (including any chambers thereof), the filter 1220, the applicator hub 1225, and out of the applicator through the tip 1230. In some embodiments, the applicator body 1205 and/or the tip 1230 may be configured to allow an operator to control the flow of the caustic formulation out of the applicator 1200. In a non-limiting example, after the compositions of embodiments herein have been released from the ampoule 1210, an operator may initiate flow of the compositions of embodiments herein out of the applicator 1200 by squeezing on the applicator body 1205, thereby generating sufficient pressure therein to force the compositions of embodiments herein to flow out through the tip 1230. In some embodiments, pressing the tip 1230 on the skin of a patient may produce capillary action sufficient to cause the compositions of embodiments herein that have been released from the ampoule 1210 and are in contact with the tip to flow through the tip and out of the applicator 1200. In some embodiments, an operator may control the rate of the flow by varying a level of force used to squeeze the sides of the applicator body 1205 and/or press the tip 1230 against the skin of the patient. In some embodiments, the applicator 1200 may include an actuator (not shown) configured to cause the compositions of embodiments herein to be dispensed from the applicator.

In some embodiments, at least one portion of the applicator may include a hydrophobic material, such as the filter 1220 and/or the tip 1230. In some embodiments the hydrophobic material is composed of polyester or co-polyester polymers, acrylic, modified acrylic (e.g., modacrylic), polypropylene, polyethylene or combinations or mixtures thereof. In some embodiments, for example, the filter may comprise a blend of polypropylene and polyester or co-polyester polymers. Non-limiting examples of hydrophobic materials may also include materials including, coated with, and/or modified by silanes, alkylsilanes, fluoroalkylsilanes, silicone, combinations thereof, and derivatives thereof. In some embodiments, materials used to form the applicator and portions thereof according to some embodiments described herein may be imparted with hydrophobic properties by coating or otherwise incorporating a hydrophobic material therein. In some embodiments, the hydrophobic portions of the applicator may operate to impede, reduce, prevent, and/or substantially prevent the compositions of embodiments herein that have been released from the ampoule from flowing through portions of the applicator and/or out of the tip of the applicator.

In some embodiments, the applicator 1200 may be configured such that the compositions of embodiments herein released from the ampoule 1210 may not be passively dispensed from the applicator 1200 without an operator applying pressure to the applicator, such as by squeezing or otherwise applying a force to the applicator body 1205. In some embodiments, the requirement for operator action (i.e., applying pressure to the applicator 1200) may be facilitated by the use of hydrophobic materials according to some embodiments. In some embodiments, the applicator 1200 may be configured such that a void may be formed within the applicator body 1205 behind a volume of the compositions of embodiments herein released from the ampoule 1210, for instance, when the applicator is orientated in a vertical, substantially vertical, or partially vertical position. In some embodiments, the void may operate as a vacuum to impede, reduce, restrict, prevent, and/or substantially prevent the flow of the compositions of embodiments herein released from the ampoule 1210 out of the applicator 1200. In this manner, the hydrophobic portion of the applicator 1200 and/or a void formed within the applicator body 1205 may operate to prevent the leakage of the compositions of embodiments herein from the applicator after the compositions of embodiments herein have been released from the frangible ampoule 1210, including when the applicator is in a position conducive to dispensing compositions of embodiments herein, such as a vertical or substantially vertical position.

The applicator 1200 may be configured to store, dispense, and apply any of the compositions of embodiments herein as part of a method of treatment for any of the conditions disclosed herein. For example, the applicator 1200 may be configured to store, dispense, and apply a topical composition comprising 2-propanol and up to about 60% w/w hydrogen peroxide. In some embodiments, the topical composition may be any composition described in embodiments herein.

While embodiments set forth herein are described in terms of “comprising”, all of the foregoing embodiments also include compositions and methods that consist of only the ingredients or steps recited or consist essentially of the ingredients and steps recited, and optionally additional ingredients or steps that do not materially affect the basic and novel properties of the composition or method.

This disclosure and embodiments illustrating the method and materials used may be further understood by reference to the following non-limiting examples.

### Example 1

A common wart (verruca) on the cheek of a 56 year old white male (Fitzpatrick Type 2 skin) was treated with a solution comprising 40% w/w hydrogen peroxide (FMC/PeroxiChem “Super D”), and 5% w/w 2-propanol (Spectrum Chemical USP Grade). After cleansing of the skin with 70% w/w 2-propanol, the solution was applied topically to the wart lesion using a flocked doe-foot shaped applicator. Using firm pressure and an application technique that was appropriate for the target lesion size, the solution was applied for approximately 20-30 seconds. After approximately 1-2 minutes, the application process was repeated. This sequence was repeated until 4 applications had been performed. There was no pain or discomfort during the procedure. Over the next several days the lesion evidenced superficial crusting and mild erythema (redness). Follow-up 2 weeks after the treatment revealed the verruca to be completely resolved with no erythema and no evidence of scarring or pigmentary change (hyperpigmentation or hypopigmentation). Follow-up 8 weeks after the treatment revealed no recurrence of the lesion and no adverse cosmetic sequelae. The subject was extremely pleased with the results.

### Example 2

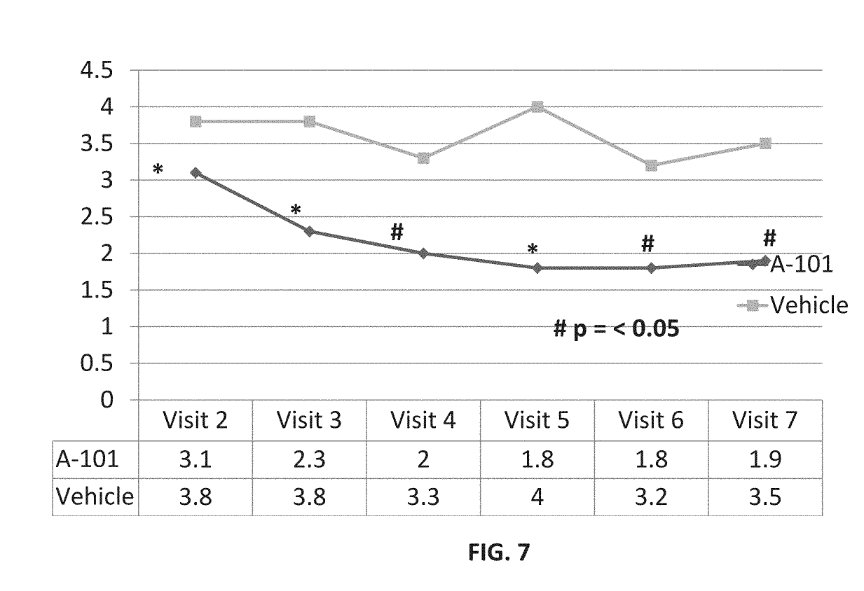

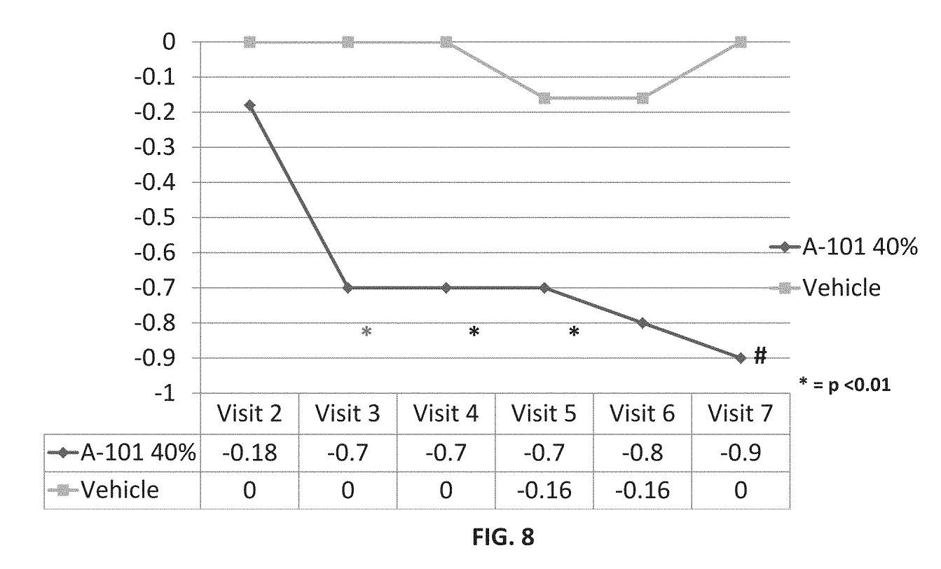

A randomized, double-blind, vehicle-controlled, parallel group study was conducted with 22 subjects, to evaluate the effectiveness of a 40% w/w topical solution of hydrogen peroxide and 5% w/w 2-propanol when applied once weekly (with a maximum of 4 applications) to treatment-naïve common warts on the extremities (not periungual or subungual) compared with a matching topical solution vehicle. The treatment protocol included (1) cleansing the target wart by rubbing with a swab/wipe wetted with 70% w/w 2-propanol; (2) wetting the supplied applicator with a quantity of study medication sufficient to wet the target wart with a thin film; (3) applying the study medication to the target wart for approximately 10 seconds using firm pressure and a circular motion; (4) absorbing excess study medication with a clean absorbent wipe (or equivalent) to minimize exposure to normal surrounding skin; (5) repeating the application procedure 3 times after a waiting period of about 20 seconds after each application. After completing the third study medication application to the target wart, the target wart was not disturbed until all visible reaction, if any, had stopped. After approximately 10 minutes, any remaining study medication was absorbed and the target wart was dried without wiping or rubbing. 17 subjects completed all four weekly treatments. All subjects tolerated the procedure well, no adverse events were reported, and local skin reactions were reported as none or mild. While no wart lesions were completely resolved at the conclusion of this pilot study (4 weekly doses)—it was expected that some of the wart lesions would show improvement, but that complete cure of the lesions might take more than four treatments (e.g., up to 10 or up to 30 treatments or more)—which is common with topical wart treatments—(e.g., over-the-counter daily salicylic acid treatments) or would require more frequent application than weekly (e.g., twice weekly or daily)—a statistically significant improvement in the lesions was demonstrated and the proof-of concept was validated. Subjects showed a statistically significant improvement in wart severity as measured by the mean Wart Improvement Assessment score by visit (FIG. 7) and by the mean change in baseline in the Wart Severity Assessment score by visit (FIG. 8). Table 1 below describes the Wart Improvement Assessment score and Wart Severity Assessment score.

TABLE 1

| Wart Severity Assessment | | Wart Improvement Assessment | |
|---|---|---|---|
| Grade | Descriptor | Grade | Descriptor |
| 0 | Clear: no clinically diagnos- | 0 | Total improvement: wart is |
| | able wart (residual erythema | | completely improved |
| | and/or fine scaling may be | | (100%); wart is cleared |
| | present) | | compared to Visit 1. |
| 1 | Mild: Barely evident clinically | 1 | Marked improvement: wart |
| | diagnosable wart, minimally | | is very much improved |
| | verrucoid, popular lesion with | | (75%) compared with |
| | no to minimal hyperkeratosis | | Visit 1 |
| 2 | Moderate: Obvious wart, | 2 | Moderate improvement: wart |
| | verrucoid papule with | | is meaningfully improved |
| | moderate hyperkeratosis | | (50%) compared to Visit 1 |
| 3 | Severe: Conspicuous wart, | 3 | Mild improvement: wart is |
| | obvious verrucoid papule | | slightly improved (25%) |
| | with severe hyperkeratosis | | compared to Visit 1 |
| | | 4 | No Change: no improvement |
| | | | compared to Visit 1 |
| | | 5 | Worse: wart has worsened |
| | | | compared to Visit 1 |

### Example 3

In vitro Drug Release and in vitro Skin Permeation studies were performed. Thirteen formulations of 40% w/w hydrogen peroxide were prepared using a stock solution of 50% w/w FMC/PeroxyChem “Super D” stabilized hydrogen peroxide as the peroxide source, with different levels of 1 and 2-propanol for assessment—as indicated in Table 2.

TABLE 2

| | Formulation and composition of excipients (% w/w w/w) | | | | | | | | | | | | |
|---|---|---|---|---|---|---|---|---|---|---|---|---|---|
| Excipient | P1-20 | P1-15 | P1-10 | P1-5 | P1-2.5 | P1-1 | P2-20 | P2-15 | P2-10 | P-2-5 | P2-2.5 | P2-1 | Control |
| H2O2 (50% w/w | 80.00 | 80.00 | 80.00 | 80.00 | 80.00 | 80.00 | 80.00 | 80.00 | 80.00 | 80.00 | 80.00 | 80.00 | 80.00 |
| w/w) | | | | | | | | | | | | | |
| 1-propanol | 20.00 | 15.00 | 10.00 | 5.00 | 2.50 | 1.00 | — | — | — | — | — | — | — |
| 2-propanol | — | — | — | — | — | — | 20.00 | 15.00 | 10.00 | 5.00 | 2.50 | 1.00 | — |

| Triethanolamine | To pH 3.5 | | | |
|---|---|---|---|---|
| Deionized water | — | q.s. 100% | — | q.s. 100% |

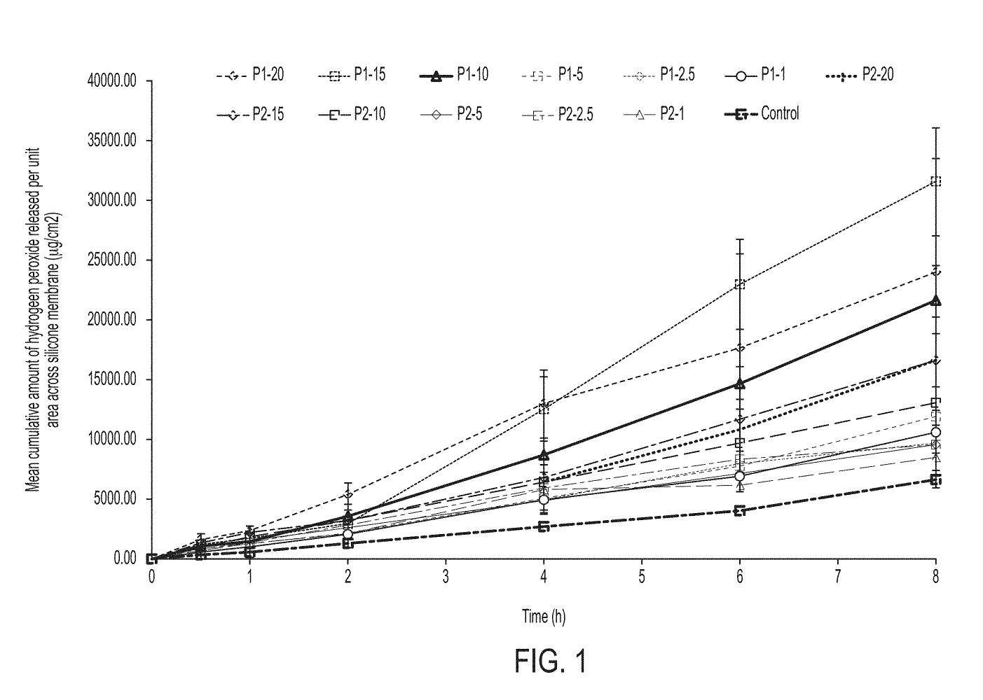

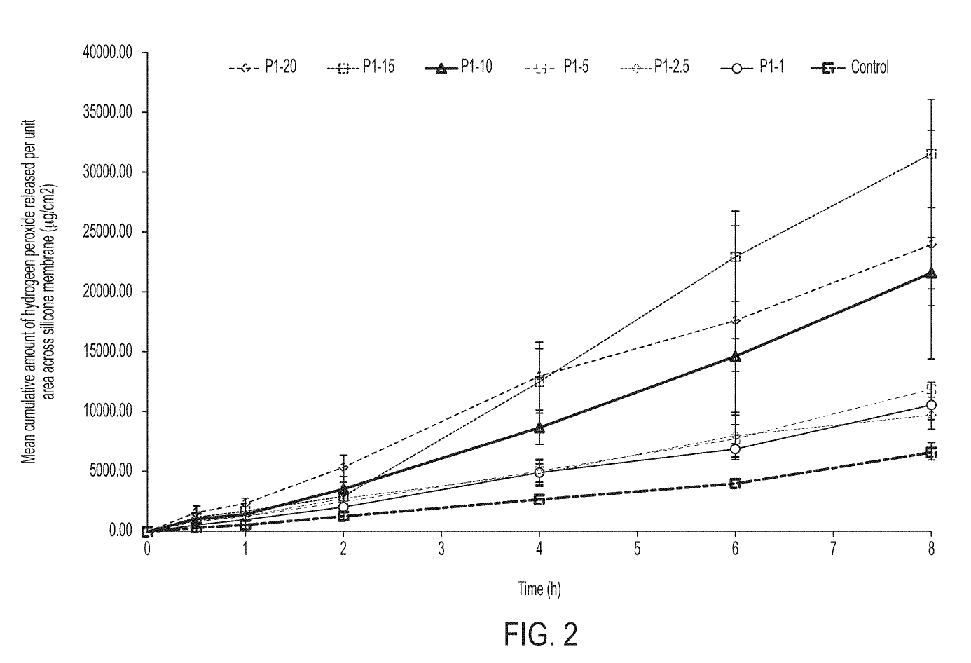

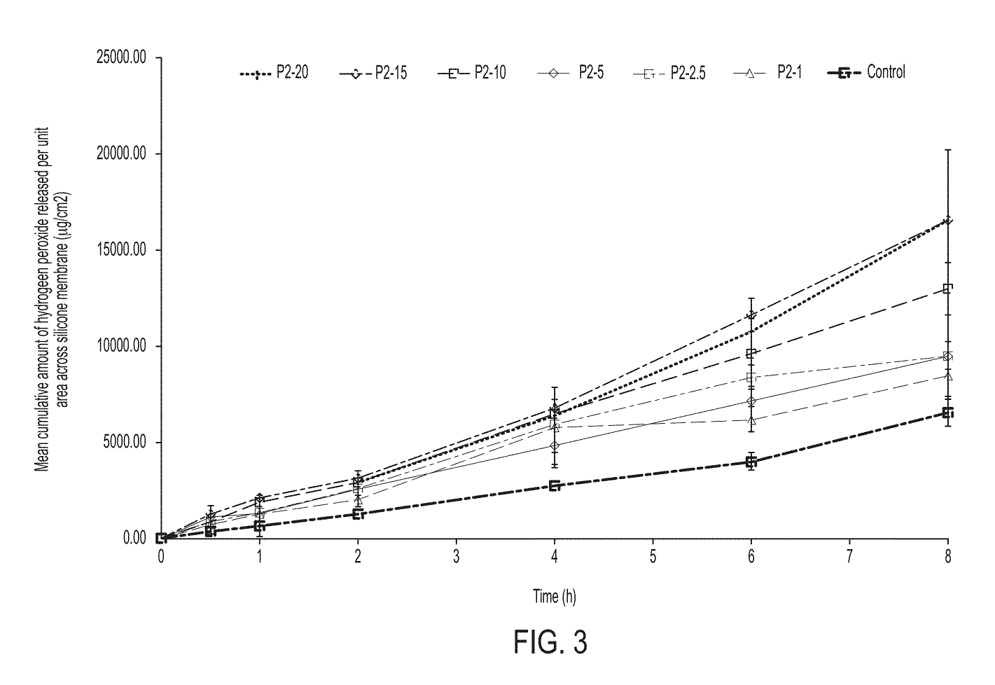

An in vitro drug release study (based on SUPAC guidelines) with the 13 formulations was performed. The receiver fluid was PBS, the synthetic membrane was silicone (selected after preliminary suitability studies), and sampling volume was 1 mL with 1 mL replenished. The measurement time points were at 0 min, 0.5 hr, 1 hr, 2 hr, 4 hr, 6 hr, and 8 hr. FIG. 1 illustrates the mean cumulative amount of hydrogen peroxide released per unit area following application of all formulations. FIGS. 2 and 3 compare the release of hydrogen peroxide between formulations containing 1- and 2-propanol, respectively, demonstrating that, in general, increasing either 1-propanol or 2-propanol content in the formulations produces an increase in the amount of hydrogen peroxide released across the silicone membrane.

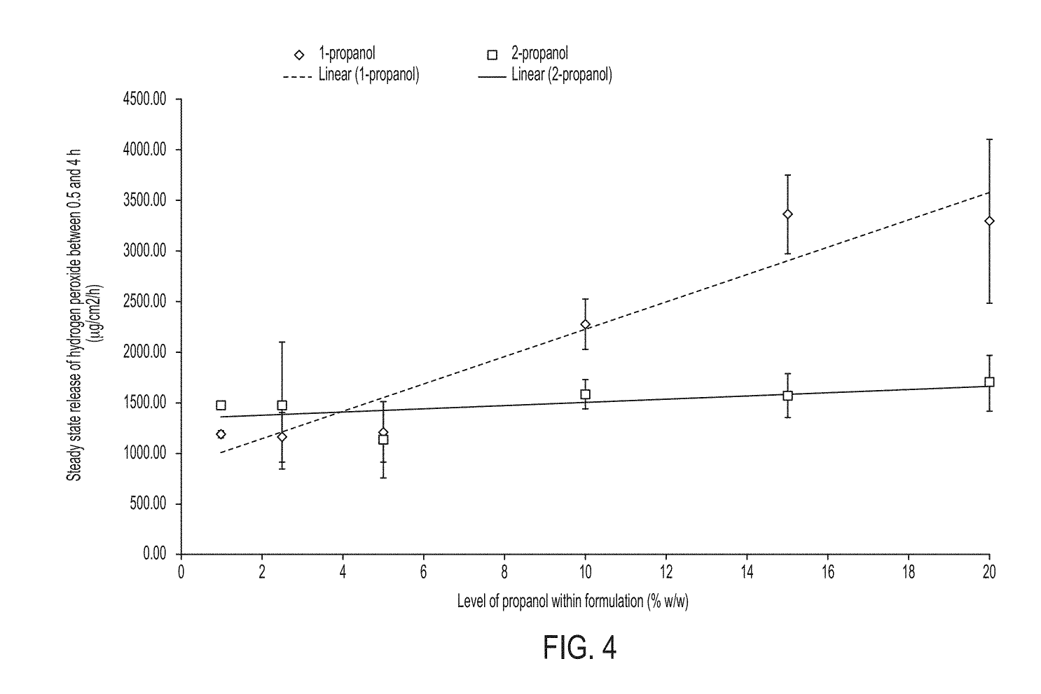

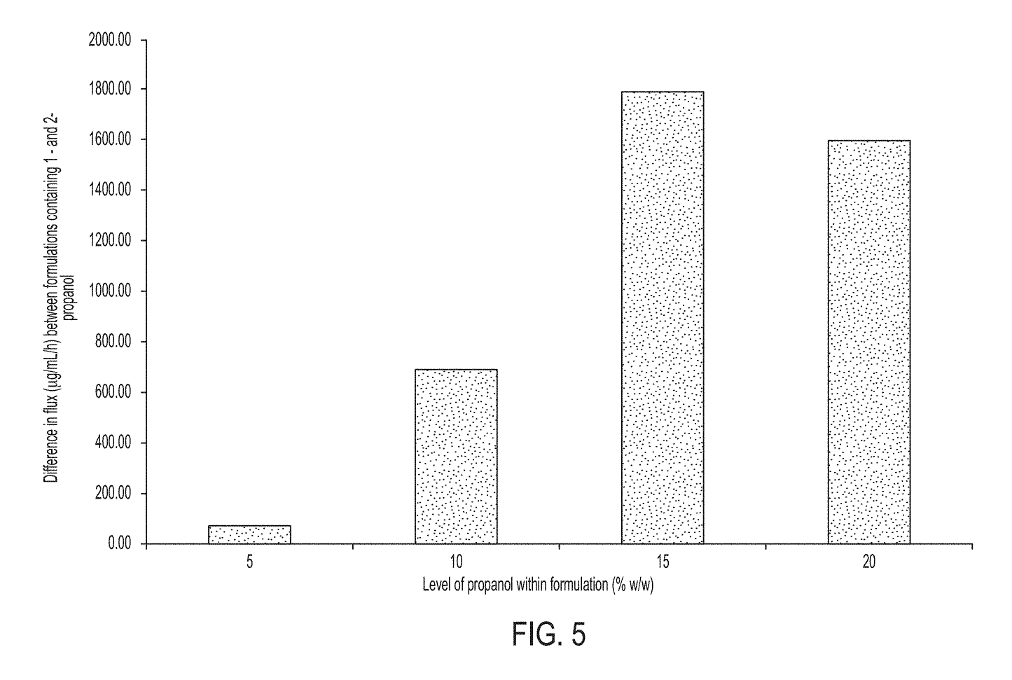

While in general, a higher amount of hydrogen peroxide is released following application of formulations containing 1-propanol compared to 2-propanol on a w/w basis, this trend is most apparent in the 1-propanol formulations and in formulations of higher concentrations of the alcohols—beginning at approximately above 5% w/w alcohol concentration—and observed to a much greater degree as the alcohol concentration increases to 10% w/w, 15% w/w and 20% w/w. This effect is illustrated by FIG. 4, which shows the steady state release of hydrogen peroxide between 0.5 h and 4 h for the different concentrations of 1-propanol and 2-propanol and FIG. 5, which shows the difference in steady state release of hydrogen peroxide between formulations containing 1-propanol and 2-propanol at levels of 5-20% w/w w/w. For formulations containing 1-propanol, there is an increase in the steady state release of hydrogen peroxide (over 0.5 to 4 h) on increasing 1-propanol content, however for formulations containing 2-propanol, the steady state release of hydrogen peroxide appears to remain essentially constant across the tested concentrations of 2-propanol.

Surprisingly, and importantly, this effect of the rate of release (0.5-4 h) of hydrogen peroxide from 1-propanol formulations being greater than corresponding 2-propanol formulations is reversed at lower concentrations of the alcohols, e.g., below approximately 5% w/w alcohol, with the rate of hydrogen peroxide release from the 2-propanol formulations at lower alcohol concentrations (e.g., <5%) being greater than the rate of hydrogen peroxide release from the 1-propanol containing formulations. Therefore, in the lower range of alcohol concentrations to be incorporated in a preferred embodiment of the formulation, which is most desirable, as minimizing the concentration of the alcohol to be added may minimize the potential impurities introduced into the peroxide formulation, it is unexpectedly discovered that 2-propanol may be preferred over 1-propanol. Without wishing to be bound by theory, it appears that the rate of release of hydrogen peroxide at lower concentrations of 2-propanol will be greater than that for the 1-propanol.

### Example 4

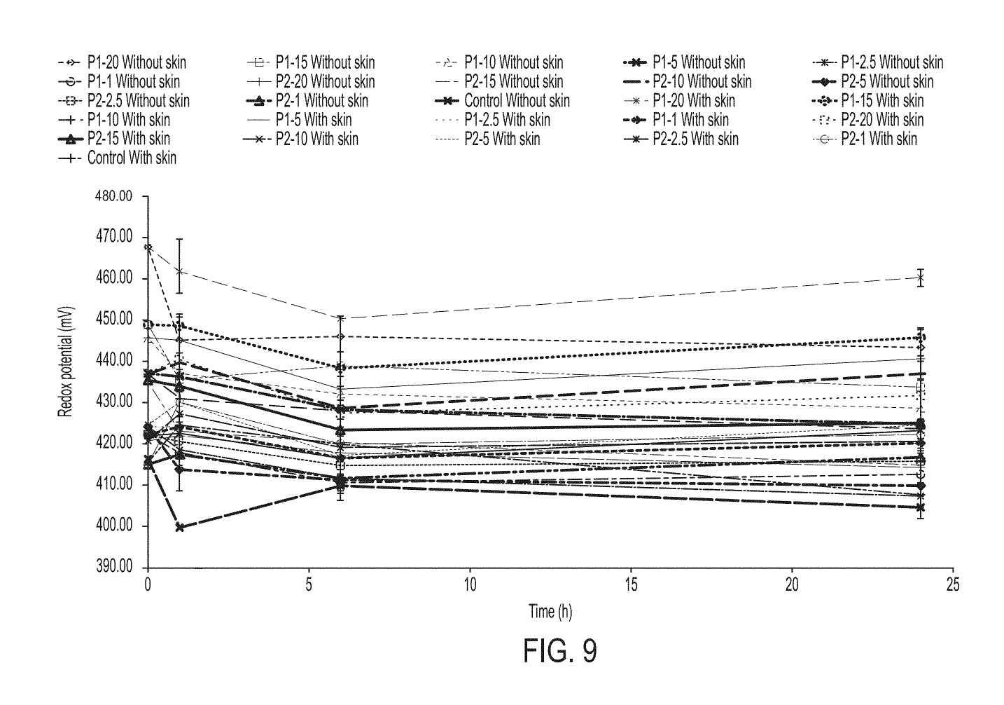

Oxidation assessment by redox potential measurement: Assessment of oxidation (by measuring redox potential) was performed on 13 prepared 40% w/w hydrogen peroxide formulations as summarized in Table 2 with and without skin at t=0, 1, 6 and 24 hr (n=3 repetitions was performed). The redox potential data of each of the 13 formulations summarized in Table 2 above is illustrated in FIG. 9.

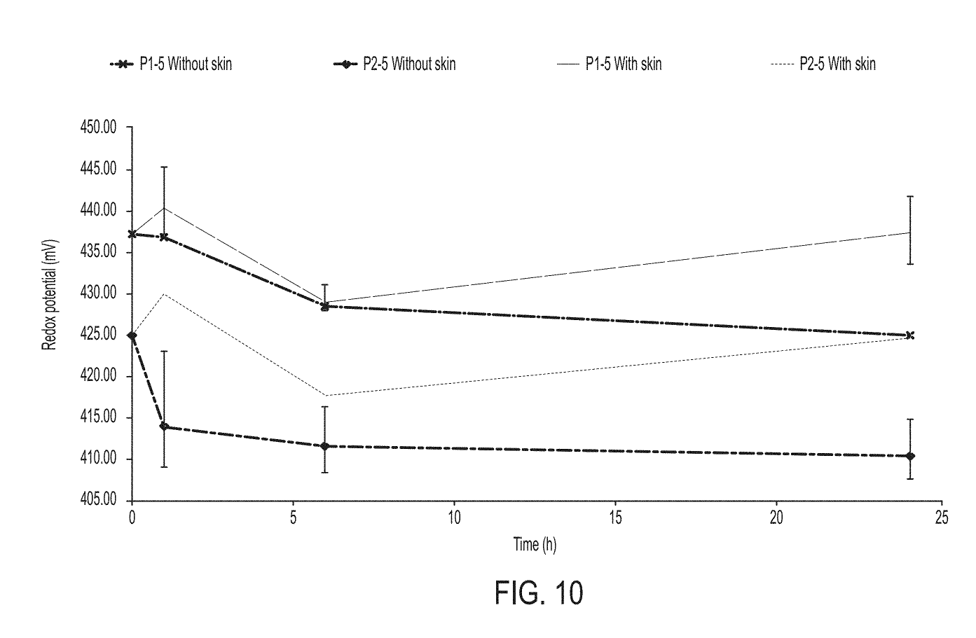

While for formulations containing less than 5% w/w propanol, the data were not statistically significant, for formulations containing 5% w/w to 20% w/w propanol, a higher (more positive) redox potential was measured for formulations containing 1-propanol when compared to 2-propanol. That is, 1-propanol, when incorporated into hydrogen peroxide formulations in this concentration range (i.e. 40%), will have a greater propensity to be oxidized than 2-propanol when incorporated into these same hydrogen peroxide formulations. A similar trend is observed in the presence of skin, where the relative redox potential between the 1-propanol and 2-propanol containing formulations is maintained. The data for the study investigating the redox potential of 40% w/w hydrogen peroxide formulations containing 5% w/w 1- or 2-propanol, both with and without skin is illustrated in FIG. 10. Thus, while it may be theoretically expected that secondary alcohols, (e.g., 2-propanol) are inherently less stable in high concentrations of hydrogen peroxide than primary alcohols, this unexpected result, in fact, demonstrates that 2-propanol is less likely to be oxidized than 1-propanol in these embodiments, and it would be preferable to incorporate 2-propanol into these solutions.

### Example 5

Surface tension analysis was performed on the 13 40% w/w hydrogen peroxide formulations with varying 1-propanol or 2-propanol concentrations from Table 2 at 37° C. (in duplicate) using Kruss Tensiometer and the Wilhelmy Plate technique. Calibration of the system using deionised water showed results within 1.0 mN/m of the literature values at 37° C. (ca. 70 mN/m). Each sample had a 30 minute run time and the result was calculated from the mean of the last 10 readings.

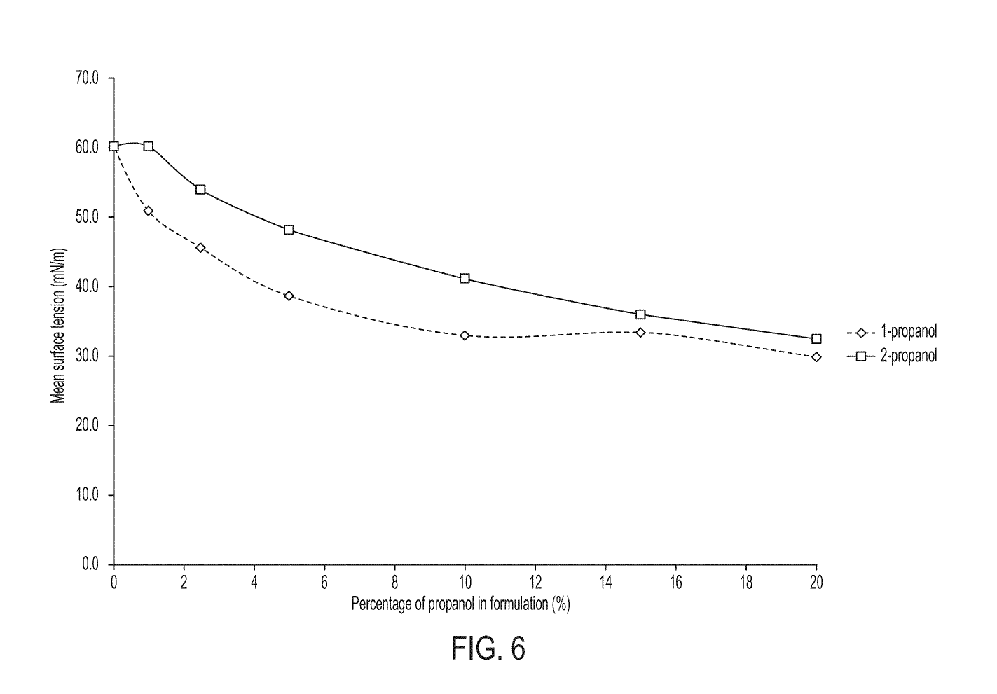

Analysis of the samples was performed in duplicate at 37±0.5° C. using a confidence level of ±1.4% w/w. Where the obtained duplicate results were outside this range, a third test was performed and the reported result was calculated using the 2 closest individual results. All data has been presented in Table 3 below and illustrated in FIG. 6.

TABLE 3

| | Surface Tension (mN/m) | | |
|---|---|---|---|
| | | Range | |
| Formulation | Results | (1st and last reading) | Rounded mean |
| P1-20 | 29.65 | 28.05-29.76 | 29.8 ± 04 |
| | 29.86 | 28.03-29.88 | |
| P1-15 | 33.44 | 29.61-33.76 | 33.3 ± 0.5 |
| | 33.22 | 29.71-33.91 | |
| P1-10 | 35.42 | 32.84-35.30 | 32.9 ± 0.5 |
| | 33.03 | 32.79-32.96 | (Tests 2 and 3) |
| | 32.80 | 32.64-32.84 | |
| P1-5 | 38.90 | 39.99-38.62 | 38.7 ± 0.5 |
| | 38.46 | 38.79-38.45 | |
| P1-2.5 | 45.95 | 45.56-45.95 | 45.7 ± 0.6 |
| | 45.34 | 46.16-45.33 | |
| P1-1 | 51.58 | 52.37-51.46 | 51.0 ± 0.7 |
| | 50.35 | 51.76-50.24 | |
| P2-20 | 32.76 | 33.89-32.59 | 32.6 ± 0.5 |
| | 32.35 | 34.03-32.20 | |
| P2-15 | 34.28 | 36.99-34.10 | 35.9 ± 0.5 |
| | 35.76 | 36.99-35.59 | (Tests 2 and 3) |
| | 36.11 | 36.94-35.96 | |
| P2-10 | 41.14 | 41.16-41.23 | 41.1 ± 0.6 |
| | 41.13 | 40.77-41.26 | |
| P2-5 | 47.75 | 48.36-47.75 | 48.3 ± 0.7 |
| | 48.95 | 48.61-49.12 | |
| P2-2.5 | 53.54 | 54.42-53.46 | 54.1 ± 0.8 |
| | 54.66 | 54.88-54.66 | |
| P2-1 | 59.85 | 62.01-59.59 | 60.2 ± 0.8 |
| | 61.87 | 62.38-61.67 | (Tests 1 and 3) |
| | 60.59 | 62.25-60.35 | |
| Control | 58.92 | 61.30-58.66 | 60.3 ± 0.8 |
| | 60.63 | 65.30-60.18 | (Tests 2 and 3) |
| | 59.99 | 65.33-59.49 | |

The data demonstrate that, in general, incorporating either 1-propanol or 2-propanol decreases the surface tension of the tested hydrogen peroxide formulations, and that this surface-tension decreasing effect is roughly proportional to the concentration of the added 1-propanol or 2-propanol. Theoretically, from a surface-tension point of view, either might be thought to be useful to incorporate into the embodiments disclosed herein.

However, the effect of the surface-tension reduction of 1-propanol on the peroxide formulations is more potent than that of 2-propanol such that in the preferred embodiments formulations containing 1-propanol will have a much lower surface tension than the corresponding 2-propanol formulations, and will be much more likely to spread out of the area of application and off the lesion, on to surrounding non-lesional skin thereby lowering the effectiveness of the therapeutic and serving as a substrate for ADH in the skin—resulting in increased irritation, erythema, and adverse cutaneous effects irritating the surrounding skin. 40%0/w/w hydrogen peroxide was employed in this illustrative study and examples, but by varying the concentration of the alcohol (e.g., 2-propanol) component in other hydrogen peroxide compositions of the embodiments described herein (e.g., in concentrations of hydrogen peroxide of 25% w/w, 32.5% w/w, 40% w/w, 42.5%), an optimal concentration of the alcohol that will produce the optimal surface tension reduction, achieve the desired clinical effect, and minimize the risk of local adverse cutaneous effects may be readily determined.

### Example 6

Solutions were prepared using stabilized hydrogen peroxide (Peroxal CG 50® Arkema, Inc.) and 2-propanol 99% w/w (Spectrum Chemical, USP Grade) to make a composition comprising 40% w/w stabilized hydrogen peroxide and 5% w/w 2-propanol and a composition comprising 25% w/w stabilized hydrogen peroxide and 5% w/w 2-propanol. The solutions were placed on stability in Type I amber borosilicate glass screw-top vials and maintained at 25° C./60% w/w relative humidity (RH); 40° C./75% w/w RH; and 5° C. (refrigerated). The 25% w/w stabilized H₂O₂/5% w/w IPA solutions remained stable (“in specification” according to ICH Harmonised Tripartite Guideline: Stability Testing of New Drug Substances and Products Q1A(R2)”, current Step 4 version, dated 6 Feb. 2003) under the 40° C. conditions at 6 months, and under both the 25° C. conditions and 5° C. conditions at 24 months. The 40% w/w stabilized H₂O₂/5% w/w IPA solutions remained stable (“in specification” according to ICH Harmonised Tripartite Guideline: Stability Testing of New Drug Substances and Products Q1A(R2)”, current Step 4 version, dated 6 Feb. 2003) under the 40° C. conditions at 6 months, under both the 25° C. conditions and 5° C. conditions at 24 months.

### Example 7

Solutions were prepared using stabilized hydrogen peroxide (Peroxal CG 50® Arkema, Inc.) and 2-propanol 99% w/w (Spectrum Chemical, USP Grade) to make a composition comprising 40% w/w stabilized hydrogen peroxide and 15% w/w 2-propanol and a composition comprising 25% w/w stabilized hydrogen peroxide and 15% w/w 2-propanol. The solutions were placed on stability in Type I amber borosilicate glass screw-top vials and maintained at 25° C./60% w/w relative humidity (RH); 40° C./75% w/w RH; and 5° C. (refrigerated). The 25% w/w stabilized H₂O₂/15% w/w IPA solutions remained stable (“in specification” according to ICH Harmonised Tripartite Guideline: Stability Testing of New Drug Substances and Products Q1A(R2)”, current Step 4 version, dated 6 Feb. 2003) under the 40° C. conditions at 6 months, and under both the 5° C. and 25° C. conditions at 24 months. The 40% w/w stabilized H₂O₂/15% w/w IPA solutions remained stable (“in specification” according to ICH Harmonised Tripartite Guideline: Stability Testing of New Drug Substances and Products Q1A(R2)”, current Step 4 version, dated 6 Feb. 2003) under the 40° C. conditions at 6 months, and under both the 5° C. and 25° C. conditions at 24 months.

### Example 8

Solutions were prepared using stabilized hydrogen peroxide (FMC/PeroxyChem “Super D 50%” (FMC/PeroxyChem) and 2-propanol 99% w/w (Spectrum Chemical, USP Grade) to make a composition comprising 45% w/w stabilized hydrogen peroxide and 5% w/w 2-propanol. The solutions were placed on stability in Type 1 amber borosilicate glass vials in stability chambers and maintained at 25° C./60% w/w relative humidity (RH), 40° C./75% w/w RH, and 5° C. (refrigerated). The 45% w/w stabilized H₂O₂/5% w/w IPA solutions remained stable (“in specification” according to ICH Harmonised Tripartite Guideline: Stability Testing of New Drug Substances and Products Q1A(R2)”, current Step 4 version, dated 6 Feb. 2003) under the under the 40° C. conditions at 6 months, and under both the 5° C. and 25° C. conditions at 12 months.

### Example 9

Solutions were prepared using stabilized hydrogen peroxide (FMC/PeroxyChem “Super D 50%” (FMC/PeroxyChem) and 2-propanol 99% w/w (Spectrum Chemical, USP Grade) to make a composition comprising 40% w/w stabilized hydrogen peroxide and 5% w/w 2-propanol. The solutions were aliquoted into heat-sealed crushable glass ampules. The solutions/ampules were placed on stability in chambers and maintained at 25° C./60% w/w relative humidity (RH); 40° C./75% w/w RH; and 5° C. (refrigerated) conditions. The 40% w/w stabilized H₂O₂/5% w/w IPA solutions remain stable (“in specification” according to ICH Harmonised Tripartite Guideline: Stability Testing of New Drug Substances and Products Q1A(R2)”, current Step 4 version, dated 6 Feb. 2003) under both the 5° C. and 25° C. conditions at 12 months thus far.

### Example 10

Solutions were prepared using stabilized hydrogen peroxide (FMC/PeroxyChem “Super D 50%” (FMC/PeroxyChem, Inc.) and 2-propanol 99% w/w (Spectrum Chemical, USP Grade) to make a composition comprising 40% w/w stabilized hydrogen peroxide and 5% w/w 2-propanol. The solutions were prepared and aliquoted into amber, Type I borosilicate glass screw-top vials. Solutions were placed on stability in chambers and maintained at 25° C./60% w/w relative humidity (RH) and 5° C. (refrigerated) conditions. The 40% w/w stabilized H₂O₂/5% w/w IPA solutions remain stable (“in specification” according to ICH Harmonised Tripartite Guideline: Stability Testing of New Drug Substances and Products Q1A(R2)”, current Step 4 version, dated 6 Feb. 2003) under both the 25° C. conditions and 5° C. conditions at 12 months thus far.

### Example 11

The following formulations were prepared using stabilized hydrogen peroxide (Peroxal CG 50® Arkema, Inc.), 2-propanol 99% w/w (Spectrum Chemical, USP Grade) comprising about 44% w/w to about 46% w/w (w/w) stabilized hydrogen peroxide, 0% w/w or 5% w/w 2-propanol (as indicated in Table 4) and a gelling agent comprising either Carbopol ETD 2020, Carbopol 974P, or Carbopol Ultrez 10, in a concentration as indicated in Table 4. Disodium EDTA was also incorporated into all formulations as a chelating agent, and pH was adjusted to between about pH 3.5-about pH 4.0. In order to maximize the level of hydrogen peroxide in the gel formulations, the pH adjustment step (using 1% w/w and 10% w/w triethylamine) was performed after the formulations were prepared and therefore the composition (% w/w weight-for-weight) in Table 4 is represented as the actual concentration of the formulations placed on stability.

TABLE 4
Gel Formulations

| Hydrogen | | |
|---|---|---|
| Peroxide | Gelling Agent | 2-propanol |
| 45.91% | 0.48% w/w Carbopol | 0% |
| | ETD 2020 | |
| 46.11% | 0.48% w/w Carbopol | 0% |
| | 974 P | |
| 45.23% | 0.47% w/w Carbopol | 0% |
| | Ultrez 10 | |
| 44.29% | 0.49% w/w Carbopol | 5% |
| | 974 P | |

The formulations were placed on stability in chambers and maintained at 25° C./60% w/w relative humidity (RH); 40° C./75% w/w RH; and 5° C. (refrigerated). The stabilized hydrogen peroxide gel formulations above were assessed for stability and based on evaluation of hydrogen peroxide recovery, pH, and Brookfield viscosity indicated that after t=6 weeks the Carbopol ETD, Carbopol 974 P, and Carbopol Ultrez 10 2020 maintained their hydrogen peroxide concentrations and their chemical & physical stability. Stability of this length of time is sufficient stability for a two-part gel formulation (or three or more part formulation with additional excipients such as 2-propanol) mixed at or immediately before the time of application where, e.g., the hydrogen peroxide and the gelling agent are kept in separate compartments (e.g., separated by a frangible, in separate vials, separate syringes) and combined or mixed when needed.

### Example 12

An applicator will have an applicator body formed from HDPE, LDPE, or an HDPE/LDPE blend, with a borosilicate glass ampoule arranged therein. A single treatment dose of a formulation of 40% w/w hydrogen peroxide and 5% w/w 2-propanol will be disposed within the ampoule. An operator will hold the applicator in their hand in a manner similar to holding a writing utensil to write. The operator will position an index finger on a first pressure area arranged on an external surface of the applicator body and the opposing thumb on a second pressure area arranged on a substantially opposite side of the applicator body. The operator will squeeze the applicator body using their index finger and opposing thumb with sufficient force to rupture the ampoule arranged within the applicator body between the first pressure area and the second pressure area. The operator will shift the position of their hand on the applicator down to a grip area on a proximal portion of the applicator body. The operator will cause the formulation to be released from a nylon flocked doe-foot shaped tip of the applicator in fluid communication with the applicator tube by squeezing on the grip area using their fingers.

A filter formed from LDPE that is coated with a silicone material configured to impart hydrophobic properties to the filter will be arranged within the applicator to filter the shards from the ruptured ampoule. The filter will prevent the flow of the formulation therethrough unless the operator applies pressure to the applicator body. The operator will control a rate of flow from the applicator onto a target application site (e.g., a skin lesion) by varying the pressure applied to the grip area. The operator will then treat the target lesion or lesions.

### Example 13

A randomized, double-blind, vehicle-controlled, parallel-group study was conducted using formulations of (i) 45% w/w hydrogen peroxide and 5% w/w 2-propanol, (ii) 40% w/w hydrogen peroxide and 5% w/w 2-propanol, and (iii) a solution vehicle in subjects with common warts. The main objective of this study was to evaluate the clinical effect of two different hydrogen peroxide/5% w/w 2-propanol solutions (45% w/w H₂O₂ and 40% w/w H₂O₂) compared with a matching vehicle when applied to common warts of the trunk & extremities. A secondary objective was to evaluate the safety of the hydrogen peroxide solutions compared with vehicle when applied to common warts. Subjects were included in the study if they had at least one target wart on the trunk or extremities that had (i) its longest axis≧3 mm and ≦10 mm, (ii) a thickness≦3 mm, and (iii) a Physician Wart Assessment (PWA) of ≧2. Subjects with both treatment naïve warts and persistent/recurrent warts were enrolled in the clinical trial.

Ninety subjects completed the study and were randomized into the following groups: Group 1: 45% w/w Solution (n=32); Group 2: 40% w/w Solution (n=31): Group 3: Vehicle Solution (n=27). Subjects were treated once weekly for a total of 8 treatments. Subjects were then brought back one week post last treatment for efficacy and safety evaluations (Visit 10). Subjects were then continued in the open-label portion of the study for an additional 11 weeks (12 weeks after the final treatment visit) in order to evaluate the duration of clinical effect.

The primary endpoint of the study was a mean change from baseline in the PWA score at Visit 10 using an analysis of covariance. The PWA is determined as shown in Table 5:

TABLE 5
PHYSICIAN WART ASSESSMENT

| Grade | Descriptor |
|---|---|
| 0 | Clear: No diagnosable wart |
| 1 | Mild: clinically diagnosable wart that is barely evident |
| 2 | Moderate: A clinically diagnosable wart that is raised |
| | and may have a slightly rough surface |
| 3 | Severe: A clinically diagnosable wart that is raised, |
| | with an obviously rough and or hyperkeratotic surface |

Comparison between vehicle and each treatment group were performed using least-square means based on the per-protocol population. Secondary endpoints included two responder analyses: the proportion of patients whose target wart is judged to be clear on the PWA at visit 10; and the proportion of patients whose target wart is judged to be clear or barely evident on the PWA at visit 10. Both responder analyses were conducted on the per protocol population using a Chi Square statistic. A further exploratory analysis was performed to see the proportion of subjects who achieved a 2 ordinal reduction in PWA score at Visit 10.

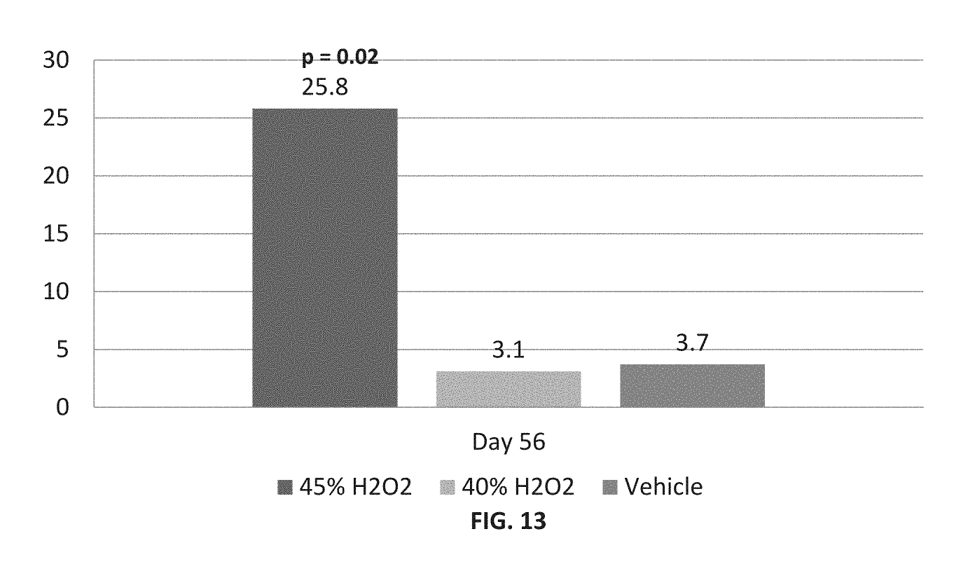

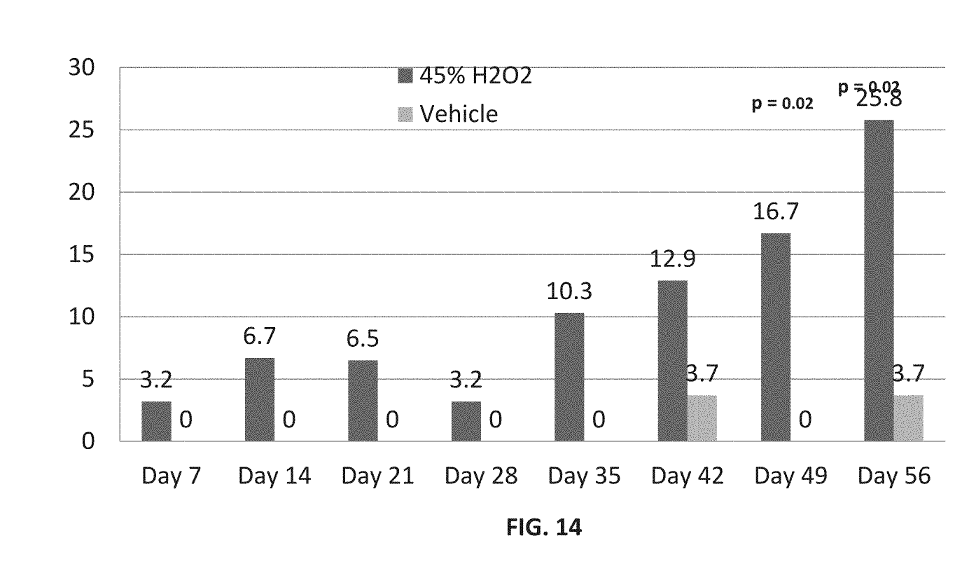

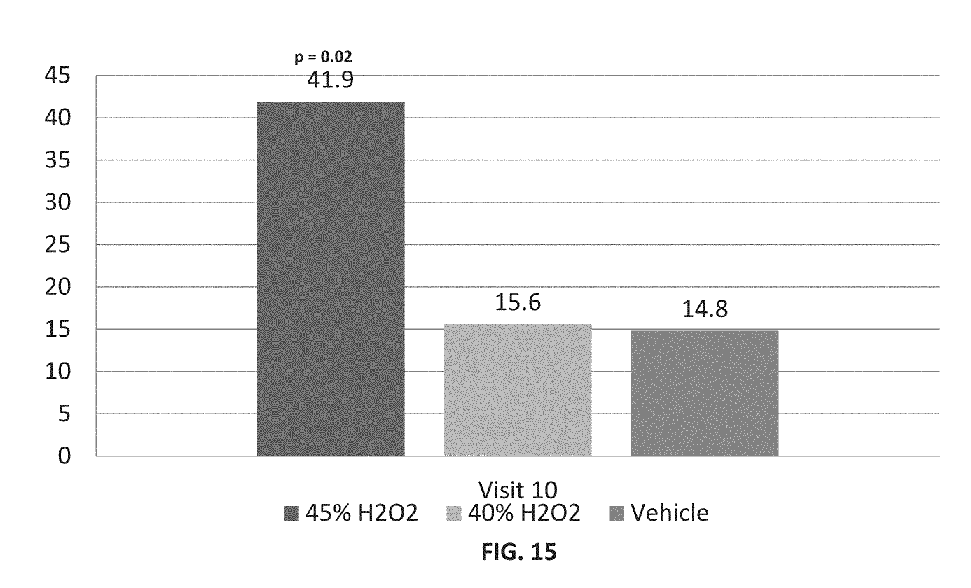

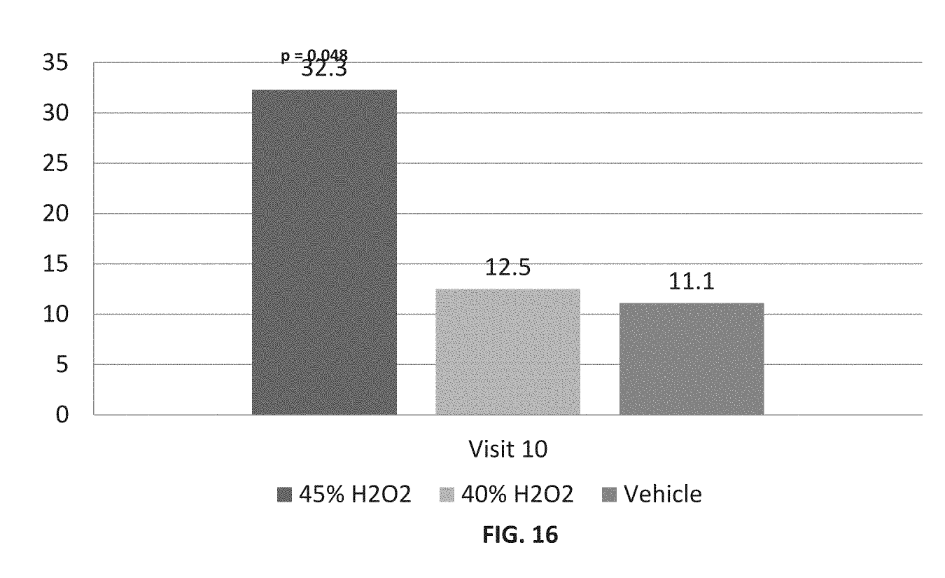

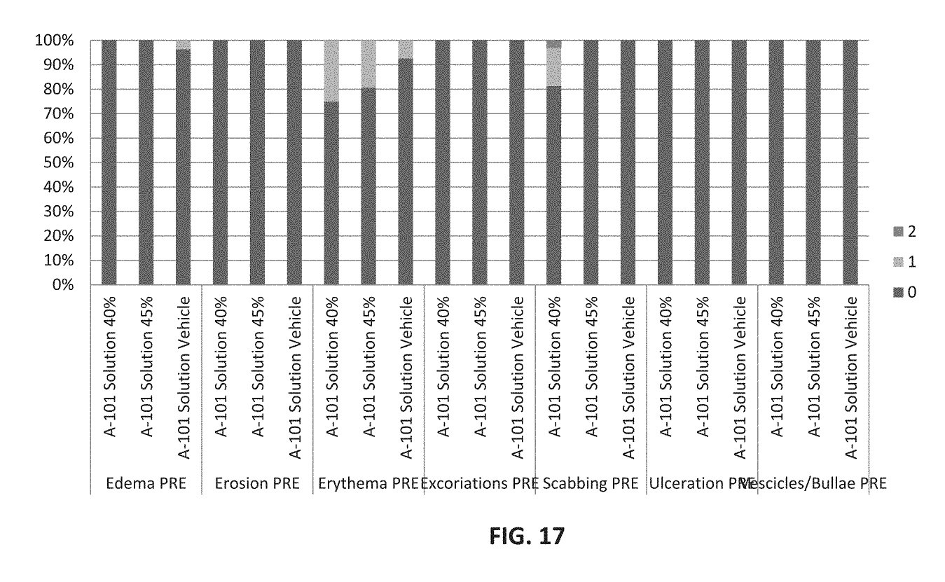

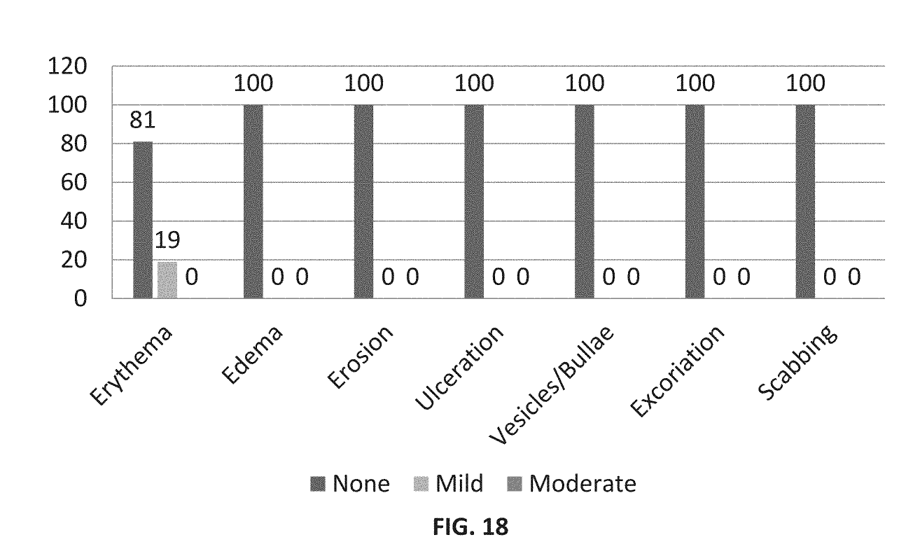

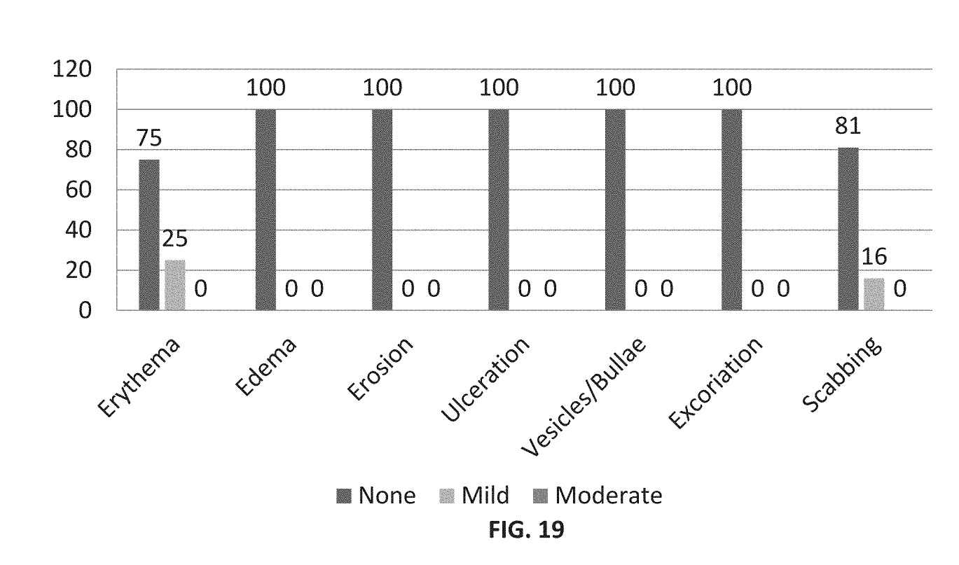

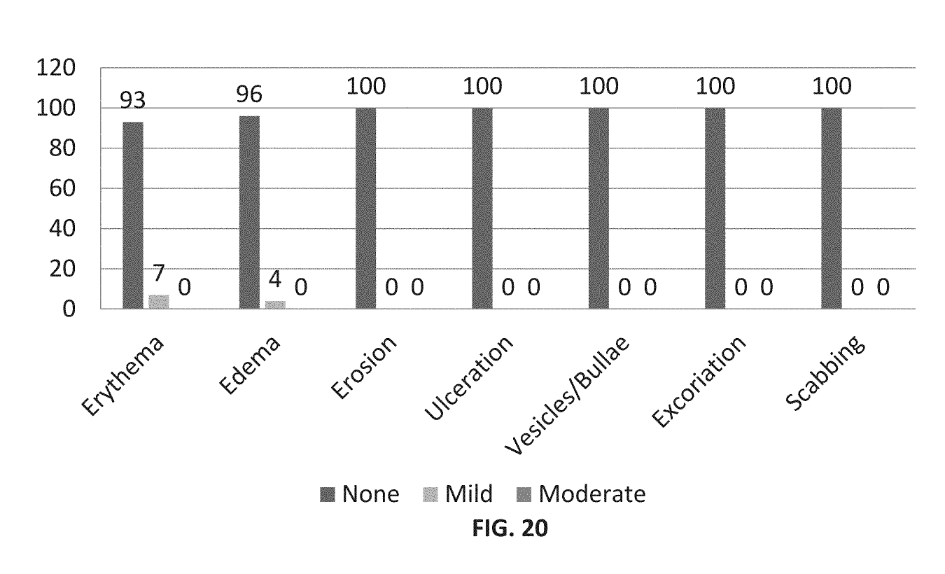

As shown in FIGS. 12 and 14, patients treated with 45% w/w hydrogen peroxide solution demonstrated a statistically significant change in PWA score (p=0.01) versus placebo. Further, as shown in FIGS. 13 and 15, the 45% w/w H₂O₂ topical solution achieved statistical significance in complete clearance (FIG. 13) of the warts and in achieving a PWA score of clear or mild (FIG. 15) at visit 10 versus placebo (p=0.02). FIG. 16 shows that a statistically significant proportion of subjects also achieved a 2 ordinal reduction in PWA score at Visit 10 with 45% w/w H₂O₂ topical solution. The local skin reactions for both the 40% w/w H₂O₂ topical solution (FIG. 19) and 45% w/w H₂O₂ topical solution (FIG. 18) were similar to vehicle (FIG. 20). As shown in FIG. 17, only mild to moderate, transient local skin reactions were observed during treatment. The treatments were extremely well tolerated, and the most frequently reported adverse event across treatment groups was mild erythema. Accordingly, the 45% w/w H₂O₂ topical solution was demonstrated to be a statistically significant safe and effective treatment for common warts. It was surprising and unexpected that the 45% w/w H₂O₂ topical solution showed such significantly positive results over the 40% w/w H₂O₂ topical solution. Whereas the 40% w/w H₂O₂ topical solution did not show a statistically significant improvement in wart severity when compared to vehicle, the 45% w/w H₂O₂ topical solution showed not only statistically significant improvement when compared to vehicle but was able to clear about 25% w/w of the warts in the treatment period and was very well-tolerated.

The final follow-up of the open-label portion of the study (Visit 13) extended an additional 11 weeks beyond the primary efficacy endpoint (Visit 10) in order to evaluate the duration of clinical effect in the treatment groups vs. the vehicle group 12 weeks after the final treatment session. The durability of the response (i.e. “clearance”—maintenance of PWA=0) at the final visit was maintained for 87.5% w/w of the subjects in the 45% w/w H₂O₂ treatment group who were responders at Visit 10. The single subject in the 40% w/w H₂O₂ treatment group who was a responder at Visit 10 remained a responder at Visit 13. These subjects demonstrate maintenance of the clinical clearance/cure of the target wart lesions at the final follow-up.

### Example 14

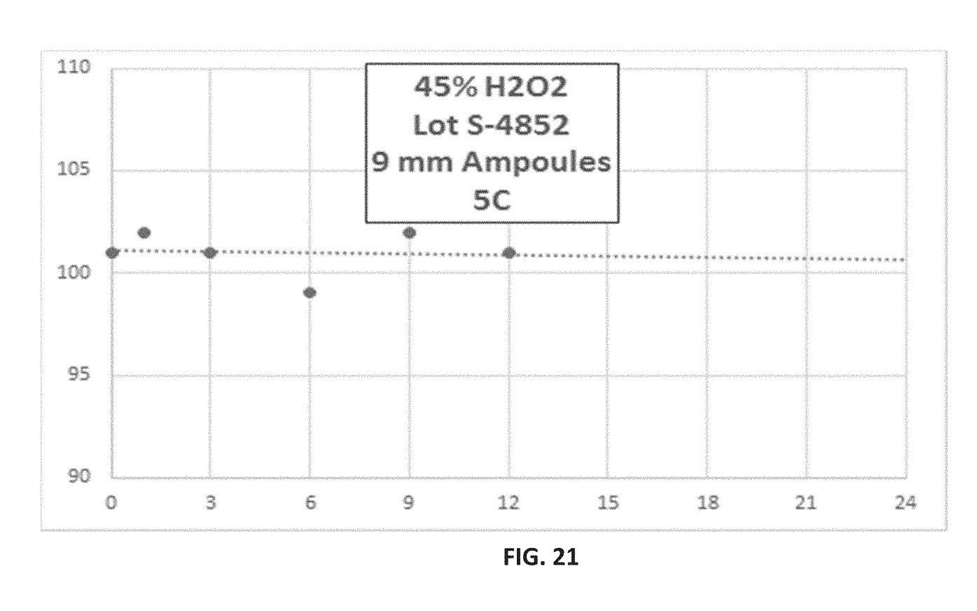

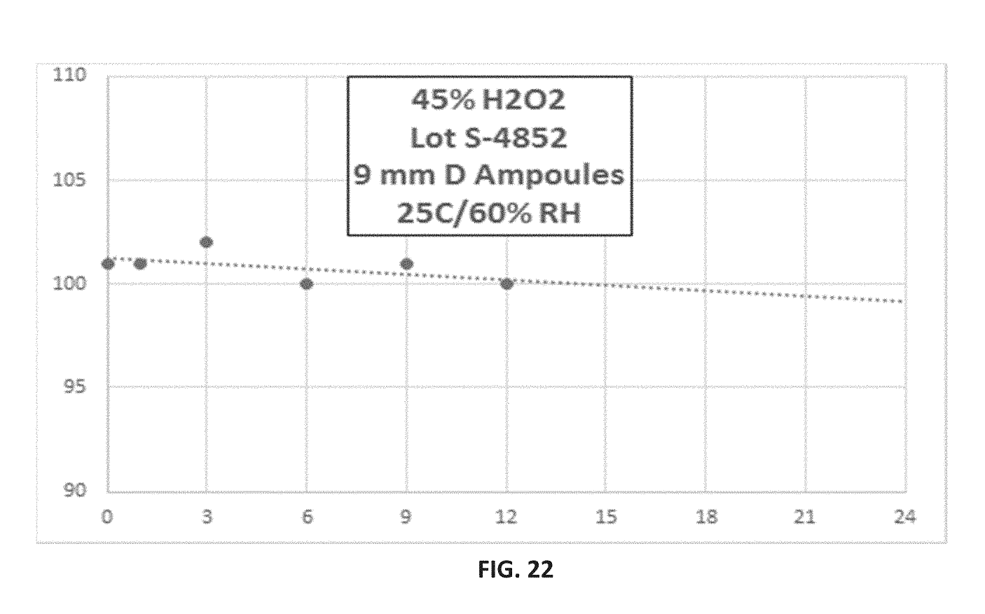

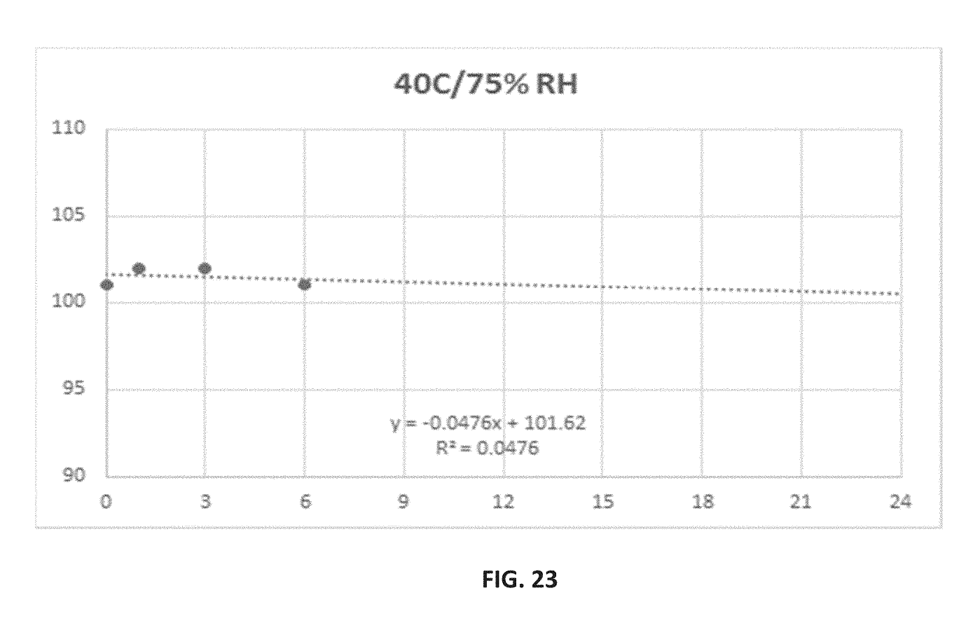

Solutions were prepared using stabilized hydrogen peroxide (FMC/PeroxyChem “Super D 50%” (FMC/PeroxyChem, Inc.)) and 2-propanol 99% w/w (Spectrum Chemical, USP Grade) to make a composition comprising 45% w/w stabilized hydrogen peroxide and 5% w/w 2-propanol. The solutions were prepared and aliquoted (2.2 ml) into 9 mm diameter ampoules. Solutions were placed on stability in chambers and maintained at 40° C./75% w/w relative humidity (RH), 25° C./60% w/w RH, and 5° C. (refrigerated) conditions. The 45% w/w stabilized H₂O₂/5% w/w IPA solutions remain stable (“in specification” according to ICH Harmonised Tripartite Guideline: Stability Testing of New Drug Substances and Products Q1A(R2)”, current Step 4 version, dated 6 Feb. 2003) under all three conditions: the 40° C. conditions at 6 months (FIG. 23), and the 25° C. conditions (FIG. 22) and 5° C. conditions (FIG. 21) at 12 months thus far.

### Example 15

A randomized, double-blind, vehicle-controlled, parallel-group study is being conducted using formulations of (i) 45% w/w hydrogen peroxide and 5% w/w 2-propanol, and (ii) a solution vehicle in both pediatric and adult subjects from 8 years old and up who have common warts. Adult subjects will self-apply the study medication and pediatric subjects will have the study medication applied by a parent or guardian. The main objective of this study is to evaluate the effectiveness of a 45% w/w hydrogen peroxide/5% w/w 2-propanol solution compared with a matching vehicle when applied by the subjects themselves for up to eight weeks, either once or twice weekly, to common warts of the trunk & extremities. The secondary objectives are to evaluate the clinical effect of 45% w/w hydrogen peroxide and 5% w/w 2-propanol solution when applied twice a week to all treated warts (target plus non-target) for up to eight weeks to evaluate the duration of response in all treated warts (target warts plus non-target warts) up to three months after the final study drug application, and to evaluate the safety of a 45% w/w hydrogen peroxide/5% w/w 2-propanol solution compared with vehicle when applied to common warts. Subjects included in the study will have from one to six target warts on the trunk or extremities that have (i) its longest axis≧3 mm and ≦8 mm, (ii) a thickness≦3 mm, and (iii) a Physician Wart Assessment (PWA) of ≧2. Pediatric (age>8 years) and adult subjects with both treatment naïve warts and persistent/recurrent warts will be enrolled in the clinical trial.

Subjects will be treated once or twice weekly for a total of up to 8 weeks (either 8 or 16 drug applications). Subjects will be brought back for regular follow-ups and evaluation during the course of the study, and will be brought back one week post the last treatment for efficacy and safety evaluations (Visit 10). Subjects will then be continued in the study for an additional 11 weeks (12 weeks after final treatment visit) in order to evaluate the duration of clinical effect including “clearance”—maintenance of PWA=0.

The primary endpoint of the study will be a mean change from baseline in the PWA score (TABLE 5) at Visit 10 using an analysis of covariance.

Comparison between vehicle and treatment group will be performed using least-square means based on the per-protocol population. Secondary endpoints will include two responder analyses: the proportion of patients whose target wart is judged to be clear on the PWA at visit 10; and the proportion of patients whose target wart is judged to be clear or barely evident on the PWA at visit 10. Both responder analyses will be conducted on the per-protocol population using a Chi Square statistic. A further exploratory analysis will be performed to see the proportion of subjects who achieved a 2 ordinal reduction in PWA score at Visit 10.

Both pediatric and adult patients treated with 45% w/w hydrogen peroxide and 5% why 2-propanol solution, both once weekly and twice weekly are expected to demonstrate a statistically significant change in PWA score versus placebo. Further, 45% w/w hydrogen peroxide and 5% w/w 2-propanol topical solution is expected to achieve statistical significance in complete clearance of the warts (PWA=0) and in achieving a PWA score of clear or mild one week after the final study medication application (Visit 10) versus placebo. Additionally, it is expected that a statistically significant proportion of subjects will also achieve a 2 ordinal reduction in PWA score at Visit 10 with 45% w/w H₂O₂ topical solution. The local skin reactions with the 45% w/w hydrogen peroxide and 5% w/w 2-propanol solution are expected to be similar to vehicle, with only mild to moderate, transient local skin reactions expected to be observed during treatment.

The treatments are expected to be well tolerated. Accordingly, it is expected that the 45% w/w hydrogen peroxide and 5% w/w 2-propanol solution will be demonstrated to be a statistically significant safe and effective treatment for pediatric and/or adult subjects with single or multiple common warts when applied for up to eight weeks once or twice weekly by the subjects themselves (or the parent or guardian for minors), and that this response will be durable-that is-maintained at the final study follow-up visit (12 weeks after the final study medication application).

It is also anticipated, as the 45% w/w hydrogen peroxide and 5% w/w 2-propanol solution may, as part of its mechanism of action, induce a local and/or systemic immune response, that common warts that do not have the 45% w/w hydrogen peroxide and 5% w/w 2-propanol solution applied directly to them may show some improvement and/or complete resolution as well. It is also anticipated to be shown that, in such instances, the response (complete clearance or improvement) is durable (as described above) as well.

Although the present invention has been described in considerable detail with reference to certain preferred embodiments thereof, other versions are possible. Therefore the spirit and scope of the appended claims should not be limited to the description and the preferred versions contained within this specification.

What is claimed is:

1. A method of treating warts or a wart associated condition in a subject in need thereof comprising administering to the subject a topical composition comprising greater than 40% w/w to about 70% w/w hydrogen peroxide.

2. The method of claim 1, wherein the topical composition comprises hydrogen peroxide in an amount of about 45% w/w, about 50% w/w, about 54% w/w, about 55% w/w, about 59% w/w, or a range of any two of these values.

3. The method of claim 1, wherein the topical composition further comprises greater than 0% w/w to about 15% w/w 2-propanol.

4. The method of claim 1, wherein the topical composition further comprises greater than 0% w/w to about 5% w/w 2-propanol.

5. The method of claim 1, wherein the warts or the wart associated condition is selected from the group consisting of common warts, filiform warts, genital warts, external genital warts, condyloma acuminata, recurrent warts, persistent warts, periungual warts, subungual warts, plantar warts, mosaic warts, recalcitrant warts, treatment naïve warts, palmoplantar warts, flat warts, epidermodysplasia verruciformis related warts, butcher's warts, oropharyngeal warts, laryngeal warts, laryngeal papillomas, laryngeal dysplasias, anogenital warts, cervical dysplasia, cervical warts, cervical neoplasia, or a combination thereof.

6. The method of claim 1, wherein administering the composition is in combination with other methods that mechanically, physically or chemically enhance penetration of the hydrogen peroxide into a lesion or that otherwise may enhance therapeutic efficacy of the composition.

7. The method of claim 6, wherein mechanically enhancing the penetration of the hydrogen peroxide into the lesion comprises sanding, filing, curetting, or debriding a treatment site of the subject.

8. The method of claim 6, wherein mechanically enhancing the penetration of the hydrogen peroxide into the lesion into the treatment site may occur before, after or concurrent with administration of the composition.

9. The method of claim 1, wherein administering the composition is once daily, twice daily, weekly, twice weekly, every other week, every other day, monthly, every two months, every three months or as directed by a packaging or a physician to achieve the desired clinical result.

10. The method of claim 1, wherein administering the composition is for at least about one week, at least about two weeks, at least about three weeks, at least about four weeks, at least about five weeks, at least about six weeks, at least about seven weeks, at least about eight weeks, at least about nine weeks, at least about ten weeks, at least about eleven weeks, at least about twelve weeks, at least about one month, at least about two months, at least about three months, at least about four months, at least about five months, at least about six months, a range of any two of these values, or until the lesion is resolved.

11. The method of claim 1, wherein the hydrogen peroxide is an active pharmaceutical ingredient grade (API) grade hydrogen peroxide.

12. The method of claim 1, further comprising administering a second composition comprising from about 25% w/w to about 70% w/w hydrogen peroxide and greater than 0% w/w to about 15% w/w 2-propanol to the subject.

13. The method of claim 1, further comprising administering a second composition comprising from about 25% w/w to about 70% w/w hydrogen peroxide and greater than 0% w/w to about 5% w/w 2-propanol to the subject.

14. The method of claim 13, wherein the second composition is administered at least about 1 day, at least about 2 days, at least about 3 days, at least about 1 week, at least about 2 weeks, at least about 3 weeks, at least about 4 weeks, at least 1 month, at least two months, at least three months, at least 4 months, at least 5 months, or at least 6 months after an initial administration of the topical composition.

15. The method of claim 1, wherein the topical composition is a solution or a gel.

16. The method of claim 1, wherein administering the composition comprises contacting a treatment site with an applicator selected from the group consisting of a sponge, a swab, a foam tipped stick, a cotton ball, a brush, a woven or non-woven fabric, roller, gauze, a glove, a pen-type applicator, a flocked, doe-foot applicator, or a combination thereof.

17. The method of claim 1, wherein administering the composition comprises contacting a treatment site with a tip of an applicator, wherein the applicator comprises: a frangible ampoule having the composition disposed therein, an applicator body having the frangible ampoule arranged therein; an applicator hub in fluid communication with the applicator body; the tip arranged at a proximal end of the applicator hub; and a filter arranged between the frangible ampoule and the tip.

18. The method of claim 17, wherein the applicator has a feature to mechanically or physically enhance penetration of the hydrogen peroxide into a lesion or otherwise enhance therapeutic efficacy of the composition.

19. The method of claim 18, wherein the feature of the applicator is intended to sand, file, curette, or debride a treatment site of the subject before administration of the topical composition.

20. A method of treating common warts in a subject in need thereof comprising topically administering to the subject a topical composition comprising greater than 40% w/w to about 70% w/w hydrogen peroxide.

21. The method of claim 20, wherein the topical composition comprises hydrogen peroxide in an amount of about 45% w/w, about 50% w/w, about 54% w/w, about 55% w/w, about 59% w/w, or a range of any two of these values.

22. The method of claim 20, wherein the topical formulation further comprises greater than 0% w/w to about 15% w/w 2-propanol.

23. The method of claim 20, wherein the topical formulation further comprises greater than 0% w/w to about 5% w/w 2-propanol.

24. The method of claim 20, wherein the topical composition comprises hydrogen peroxide in an amount of about 45% w/w and 2-propanol in an amount of about 5% w/w.

25. The method of claim 20, wherein administering the topical composition is once weekly or twice weekly.

26. The method of claim 20 further comprising administering an additional treatment modality comprising a second hydrogen peroxide formulation, an adjuvant, an inhibitor, tape-stripping, cryotherapy, electrosurgery, curettage, laser therapy, salicylic acid, trichloroacetic acid, podophyllin, cantharidin, 5-fluorouracil, bleomycin, topical imiquimod, intralesional candida antigen, topical squaric acid dibutyl ester, oral cimetidine, surgical lesion removal, electrodesiccation, lasers of various wavelengths (ablative and non-ablative), radio-frequency ablation, dermabrasion, curettage, surgical excision, an ablative, a chemical peeling agent, disturbing the surface of the lesion, decreasing the thickness or size or overall volume of the lesion, increasing the surface area of the lesion, or a combination thereof.

27. A kit for treating warts comprising a container comprising greater than 40% w/w to about 70% w/w hydrogen peroxide and 2-propanol in an amount greater than 0% to about 5%.

28. The kit of claim 27 further comprising an applicator.

29. The kit of claim 28, wherein the applicator has a feature to mechanically enhance penetration of the hydrogen peroxide into a lesion or that otherwise may enhance therapeutic efficacy of the composition.

30. The kit of claim 27 further comprising a material or an applicator having a feature intended to sand, file, currete, or debride a treatment site of the subject.
